(19) United States
(12) Patent Application Publication
(10) Pub. No.: US 2023/0277607 A1
(43) Pub. Date: Sep. 7, 2023

(54) Natural non-pathogenic microorganisms capable of associating glycolipids or lipopeptides and use thereof

(71) Applicant: ACARYON GMBH, Berlin (DE)

(72) Inventors: Philippe Ulsemer, Schildow (DE); Kawe Toutounian, Berlin (DE)

(73) Assignee: ACARYON GMBH, Berlin (DE)

(21) Appl. No.: 17/768,138

(22) PCT Filed: Jul. 14, 2020
(86) PCT No.: PCT/EP2020/069910

§ 371 Date: Apr. 11, 2022

Related U.S. Application Data
(60) Provisional application No. 62/914,912, filed on Oct. 14, 2019.

(51) Int. Cl. A61K 35/742; A61K 35/68; A61K 35/745; A61K 35/747; C12N 11/16; C12N 1/20

(52) U.S. Cl. CPC A61K 35/742; A61K 35/68; A61K 35/745; A61K 35/747; C12N 11/16; C12N 1/20

(57) ABSTRACT

The present invention relates to modified non-pathogenic microorganisms (e g bacteria, yeasts or fungi) comprising a cell and a heterologous lipid carrier, wherein said lipid carrier comprises a) a lipid portion, wherein said lipid portion is at least partially associated with an exterior surface of said cell of said modified microorganism and wherein said lipid portion comprises a ceramide-like glycolipid moiety and/or a fatty acid moiety, and wherein said lipid carrier further comprises b) a non-lipid portion, wherein said microorganism is capable of locating and/or displaying said non-lipid portion or fragment thereof onto the exterior surface of said cell, wherein said cell of said modified microorganism does not comprise a mycomembrane and wherein said heterologous lipid carrier is not alpha-galactosylceramide. A composition comprising one or more of the modified microorganism and a vaccine or adjuvant comprising the microorganism or said composition are also subject to the present invention and are among others, useful for the development of oral vaccines, oral drug delivery systems and anti-infectious agents as well as for various applications and/or treatments. Furthermore, the present invention relates to a method for producing or isolating said modified microorganism and a method for screening for a lipid carrier, growth medium, loading medium, loading conditions, or growth conditions.

### FIELD OF THE INVENTION

The present invention relates to modified non-pathogenic microorganisms (e.g. bacteria, yeasts or fungi) comprising a cell and a heterologous lipid carrier, wherein said lipid carrier comprises a) a lipid portion, wherein said lipid portion is at least partially associated with an exterior surface of said cell of said modified microorganism and wherein said lipid portion comprises a ceramide-like glycolipid moiety and/or a fatty acid moiety, and wherein said lipid carrier further comprises b) a non-lipid portion, wherein said microorganism is capable of locating and/or displaying said non-lipid portion or fragment thereof onto the exterior surface of said cell, wherein said cell of said modified microorganism does not comprise a mycomembrane and wherein said heterologous lipid carrier is not alpha-galactosylceramide. A composition comprising one or more of the modified microorganism and a vaccine or adjuvant comprising the microorganism or said composition are also subject to the present invention and are among others, useful for the development of oral vaccines, oral drug delivery systems and anti-infectious agents as well as for various applications and/or treatments. Furthermore, the present invention relates to a method for producing or isolating said modified microorganism and a method for screening for a lipid carrier, growth medium, loading medium, loading conditions, or growth conditions.

### BACKGROUND OF THE INVENTION

Non-pathogenic microorganisms especially food graded microorganisms displaying a specific structure (e.g. oligosaccharide, peptide, proteins) on their surface have a broad application potential in food and medicine like oral vaccines, oral drug delivery systems and anti-infectious agents. Same also holds true for bacteria of the skin and other sites such urogenital tract, respiratory system or oral cavity.

Standard procedures to produce non-pathogenic microorganisms displaying a specific structure (e.g. oligosaccharide or peptide) on their surface are mainly based on genetic modifications that result in recombinant microorganisms (Genetically Modified Organisms or GMOs). Alternatively, non-GMO approaches are using fusion proteins composed of a peptide of interest bound to a protein-domain (anchor-domain) known to anchor itself to the surface of suitable microorganisms (Michon et al., 2016). However, when loaded on live microorganisms, the stability and integrity of the fusion protein may be affected by natural proteolytic activities of the said microorganism (Ganesh et al., 2014). Furthermore, potential immunogenic properties of the anchor-domain may significantly limit the application potential of this methodology (Schmidt et al., 2011).

Thus, as is evident from the above, to obtain a non-pathogenic microorganism displaying a non-endogenous structure (e.g. peptide or carbohydrate) to the environment, the prior art provides either recombinant microorganisms, the use of which is related to significant safety concerns, or recombinant fusion proteins that may be loaded on non-pathogenic microorganisms. Currently, no non-pathogenic microorganisms are known to be able to bind a heterologous lipid carrier (e.g. glycolipid or lipopeptide) in such a way so that the carried moiety (e.g. carbohydrate or peptide) is displayed to the environment.

However, such a strategy would be particularly promising to generate non-pathogenic microorganisms presenting oligosaccharides, peptides or chemical molecules of interest to the environment without employing genetic modifications of the non-pathogenic microorganism. The application's potential includes, among others, the development of oral vaccines, oral drug delivery systems, anti-infectious agents and functional foods.

### SUMMARY OF THE INVENTION

The present invention relates in a first aspect to a modified microorganism comprising a cell and a heterologous lipid carrier, said lipid carrier comprising:
a) a lipid portion, wherein said lipid portion is at least partially associated with an exterior surface of said cell of said modified microorganism, wherein said lipid portion comprises a ceramide-like glycolipid moiety and/or a fatty acid moiety; preferably said exterior surface of said cell comprising: a cell wall and/or a cell membrane and/or an outer cell membrane and/or a polysaccharide; further preferably said lipid portion is at least partially incorporated and/or adhered and/or bound to said exterior surface of said cell; and
b) a non-lipid portion, wherein said microorganism is capable of locating and/or displaying said non-lipid portion or fragment thereof onto the exterior surface of said cell, preferably said non-lipid portion comprising a carbohydrate moiety, a lipopeptide moiety, a linker, a chemical compound, or a peptide moiety;
optionally, said modified microorganism further comprises a heterologous steroid moiety; preferably said steroid moiety is cholesterol, a derivative or analog thereof, wherein said cell of said modified microorganism does not comprise a mycomembrane, preferably said mycomembrane is located in the exterior surface of said cell of said modified microorganism, and wherein said heterologous lipid carrier is not alpha-galactosylceramide.

Preferably, the lipid-portion further comprises an amino alcohol moiety, more preferably the amino alcohol moiety is sphingosine.

The present invention may also comprise said modified microorganism as defined elsewhere herein, wherein the carbohydrate of said non-lipid portion is a sialic acid residue.

The present invention may also comprise said modified microorganism as defined elsewhere herein, wherein the association of the heterologous lipid carrier with an exterior surface of said cell of said modified microorganism resists a treatment with 0.3% bile salts, optionally in combination with pancreatine juice in PBS or DPBS for at least 1 hour at least 37° C.

The present invention may also envisage said modified microorganism as defined elsewhere herein, wherein resistance means that at least 80% of the heterologous lipid carrier associated with an exterior surface of said cell of said modified microorganism remains associated after a treatment with 0.3% bile salts, optionally in combination with pancreatine juice in PBS or DPBS for at least 1 hour at least 37° C.

The present invention may also encompass said modified microorganism as defined elsewhere herein, wherein said lipid carrier has one or more of the following characteristics:
i) comprising a glyceride moiety; preferably said glyceride moiety comprises at least one fatty acid, further preferably said lipid portion of said lipid carrier comprises said glyceride moiety;
ii) comprising a ceramide moiety, wherein preferably:
   said ceramide moiety is composed of an amino alcohol (e.g. sphingosine) and/or a fatty acid, further preferably said lipid portion of said lipid carrier comprises said ceramide moiety;
iii) comprising a carbohydrate moiety; preferably said non-lipid portion of said lipid carrier comprises said carbohydrate moiety; further preferably said carbohydrate moiety is ß- or α-linked to said ceramide moiety, further preferably said carbohydrate is not a monosaccharide or a disaccharide moiety; further preferably said carbohydrate is selected from the group consisting of: an oligosaccharide and a polysaccharide, most preferably the first sugar of the said carbohydrate is galactose, a glucose, a mannose, a xylose, a neuraminic acid, a N-acetyl glucosamine, N-acetyl galactosamine or a galacturonic acid;
iv) comprising one or more polypeptides (e.g. a heterologous recombinant or fusion polypeptide, e.g. a glycosylated polypeptide or an immunologically active polypeptide), preferably said non-lipid portion comprising said one or more polypeptides; further preferably said one or more polypeptides is an enzyme, a cytokine or a chemokine, a peptidomimetic compound, an antigen, an antibody, a fragment or derivative thereof;
v) comprising a pharmaceutically active compound; preferably said non-lipid portion comprising said pharmaceutically active compound;
vi) is not comprising a recombinant and/or fusion polypeptide, preferably said recombinant and/or fusion polypeptide is obtained by the means of artificial genetic manipulation;
vii) is not expressed or synthetized by said microorganism;
viii) is at least partially expressed or synthetized by said microorganism, preferably said microorganism is capable of expressing or synthetizing a ceramide or sphingolipid moiety;
ix) said lipid portion is not comprising a polypeptide;
x) is not comprising a transmembrane polypeptide or a polypeptide membrane anchor domain;
xi) is not susceptible to proteolysis;
xii) is not immunogenic to a mammalian host, preferably said mammalian host is human;
xiii) is immunogenic to a mammalian host, preferably said mammalian host is human;
xiv) is not covalently bound to said cell membrane of said cell of said modified microorganism;
xv) comprising a glycolipid;
xvi) comprising a lipopeptide (e.g. a glycosylated lipopeptide).

The present invention may also comprise said modified microorganism as defined elsewhere herein, wherein said lipid carrier is selected from the group consisting of:

i) Monosialotetrahexosylganglioside (GM1) or Monosialotetrahexosylganglioside red (GM1red) having the following formula

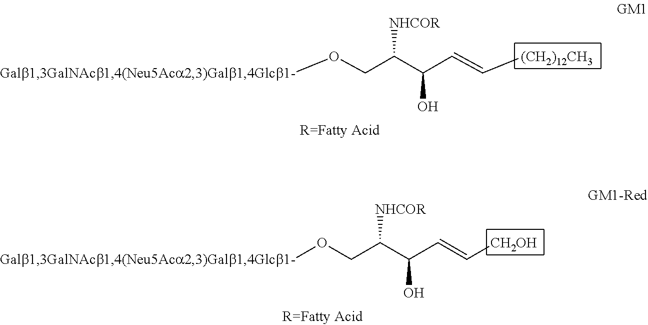

ii) Globotriaosylceramide (Gb3), a GM1-Gb3 chimera having the formula

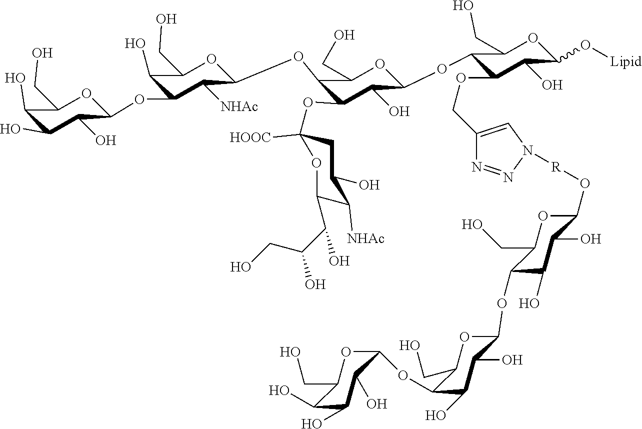

iii) Ganglioside GD1a,
iv) Gangliosides GM2, GD2, GD1b, GT1b, GT1c, GQ1c, GA1, GM1b,
v) Gangliosides GM3, GD3 and GT3,
vi) Gangliosides Gb4, Blood Group Type I, Type 2, Blood Group A, Blood Group B, Blood Group H, Blood Group H Type 1, Blood Group H Type 2, Blood Group H Type 3, Lewis y, Lewis a, Lewis b, Lewis x, H, Sialyl Lewis x, Sialyl Lewis a, Sialyl Lewis b, Sialyl Lewis x, alpha Gal epitope, Gal a1-3Galß1-4GlacNAc, Gal(α1-4)Gal(ß1-4)GlcNAc-R, Gal(α 1-4)Gal(ß1-4)Glc, NAc-(ß1-3)Gal(ß1-4)Glc-R, Gal(α 1-4)Gal(ß1-4)GlcNAc(ß1-2) Man-R
vii) any one of the following:

| Name | Structure |
|---|---|
| 2-6 Sialyl i-Lewis x | Neu5Ac(α 2-6)Gal(β1-4)GlcNAc(β1-3)Gal(β1-4)[Fuc(α 1-3)]GlcNAc(β1-)-R |
| 3′-Sulfo Lewis a | HSO3(-3)Gal(β1-3)[Fuc(a1-4)]GlcNAc(β1-)-R |
| 3′-Sulfo Lewis x | HSO3(-3)Gal(β1-4)[Fuc(a1-3)]GlcNAc(β1-)-R |
| 6,6′-Disulfo Sialyl Lewis x | Neu5Ac(α 2-3)[HSO3(-6)]Gal(β1-4)[Fuc(α 1-3)][HSO3(-6)]GlcNAc(β1-)-R |
| 6-Sulfo Lewis x | Gal(β1-4)[Fuc(α 1-3)][HSO3(-6)]GlcNAc(β1-)-R |
| 6′-Sulfo Sialyl Lewis x | Neu5Ac(α 2-3)[HSO3(-6)]Gal(β1-4)[Fuc(α 1-3)]GlcNAc(β1-)-R |
| 6^{(GlcNAc)}-su-SLe^{x} | Neu5Acα2-3Galβ1-4(Fucα1-3)(6-O-Su)GlcNAcβ-R |
| 6′-Sia-6-Su-LacNAc | Neu5Acα2-6Galβ1-4(6-O-Su)GlcNAcβ-R |
| 6-Su-3′SiaLe^{c} | Neu5Acα2-3Galβ1-3(6-O-Su)GlcNAcβ-R |
| 6-Su-3′SLN | Neu5Acα2-3Galβ1-4(6-O-Su)GlcNAcβ-R |
| 3′SLN(Gc) | Neu5Gcα2-3Galβ1-4GlcNAcβ-R |
| 6′SLN(Gc) | Neu5Gcα2-6Galβ1-4GlcNAcβ-R |
| GlcNAcβ3′LacNAc | GlcNAcβ1-3Galβ1-4GlcNAcβ-R |
| Isomaltotriose | Glcα1-6Glca1-6Glcβ-R |
| Chitotriose | GlcNAcβ1-4GlcNAβ1-4GlcNAcβ-R |
| alpha Gal epitope, | Gal(α 1-3)Gal(β1-4)GlcNAc-R |
| Arthro | GlcNAcβ1,3Manβ1,4Glcβ-R |
| Aₜᵣᵢ | GalNAcα1-3(Fucα1-2)Galβ-R |
| Bₜᵣᵢ | Galα1-3(Fucα1-2)Galβ-R |
| Gal₂3,4-GlcNAc | Galβ1-4(Galβ1-3)GlcNAcβ-R |
| asialo-GM1,GA1 | DGalp(β1-3)DGalpNAc(β1-4)DGalp(β1-4)DGlcp(β1-1)-R |
| asialo-GM2,GA2 | DGalpNAc(β1-4)DGalp(β1-4)DGlcp(β1-1)-R |
| Blood Group A | GalNAc(α 1-3)[Fuc(α 1-2)]Gal(β1-)-R |
| Trisaccharide | |
| Blood Group A | GalNAc(α 1-3)[Fuc(α 1-2)]Gal(β1-3)GlcNAc(β1-)-R |
| Type 1 | |
| Blood Group A | GalNAc(α 1-3)[Fuc(α 1-2)]Gal(β1-3)[Fuc(α 1-4)]GlcNAc(β1-)-R |
| Type 1 (difucosyl) | |
| Blood Group A | GalNAc(α 1-3)[Fuc(α 1-2)]Gal(β1-4)GlcNAc(β1-)-R |
| Type 2 | |
| Blood Group A | GalNAc(α 1-3)[Fuc(α 1-2)]Gal(β1-4)[Fuc(α 1-3)]GlcNAc(β1-)-R |
| Type 2 (difucosyl) | |
| Blood Group A | GalNAc(α 1-3)[Fuc(α 1-2)]Gal(β1-3)GalNAc(α 1-)-R |
| Type 3 | |
| Blood Group A | GalNAc(α 1-3)[Fuc(α 1-2)]Gal(β1-3)GalNAc(β1-)-R |
| Type 4 | |
| Blood Group B | Gal(α 1-3)[Fuc (α 1-2)]Gal(β1-3)GlcNAc(β1-3)Gal-R, |
| Blood Group B | Gal(α 1-3)[Fuc(α 1-2)]Gal(β1-4)GlcNAc(β1-)-R |
| Type 2 | |
| Blood Group H | Fuc(α 1-2)Gal(β1-3)GlcNAc(β1-)-R |
| Type 1, | |
| Blood Group H | Fuc(α 1-2)Gal(β1-4)GlcNAc(β1-)-R |
| Type 2, | |
| Blood Group H | Fuc(α 1-2)Gal(β1-3)GalNAc(α 1-)-R |
| Type 3, | |
| Blood Group H, | Fuc(α 1-2)Gal(β1-)-R |
| Sia6′H (type 2) | Neu5Acα2-6(Fucα1-2)Galβ1-4GlcNAcβ-R |
| 6-LacNAc-TF | Galβ1-4GlcNAcβ1-6(Galβ1-3)GalNAcα-R |
| C-Series Ganglio- | R-Gal(β1-3)GalNAc(β1-4)[Neu5Ac(α 2-8)Neu5Ac(α 2-8)Neu5Ac(α 2- |
| sides Oligo- | 3)]Gal(β1-4)Glc(β1-1)-R |
| saccharide/Ganglio- | |
| tetraosyl Core | |
| Structure | |
| C-Series Ganglio- | Neu5Ac(α 2-8)Neu5Ac(α 2-8)Neu5Ac(α 2-3)Gal-R |
| sides Oligo- | |
| saccharide/Hemato- | |
| or Ganglio-Type | |
| Cyclic Sialyl 6-Sulfo Lewis x | cyclicNeu5Ac(α 2-3)Gal(b1-4)[Fuc(α 1-3)][HSO3(-6)]GlcNAc-R |
| Dimeric Lewis x | Gal(β1-4)[Fuc(α 1-3)]GlcNAc(β1-3)Gal(b1-4)[Fuc(α 1-3)]GlcNAc(β1-)-R |
| Disialyl Lewis a | Neu5Ac(α 2-3)Gal(β1-3)[Neu5Ac(α 2-6)][Fuc(α 1-4)]GlcNAc(β1-)-R |
| Disialyl Lewis c | Neu5Ac(α 2-3)Gal(β1-3)[Neu5Ac(α 2-6)]GlcNAc(β1-)-R |
| F1 Alpha | Gal(b1-4)GlcNAc(b1-6)GalNAc(α 1-)Ser/Thr |
| fucosyl GM1 | (Fucal-2Galβ1-3GalNAcβ1-4[NeuAca2-3]-Galβ1-4Glcβ1-I-R |
| GA1: (Gg4Cer) | Galβ1,3GalNAcβ1,4Galβ1,4Glcβ1-R |
| GA2: (Gg3Cer) | GalNAcβ1,4Galβ1,4Glcβ1-R |
| Gal a1-3Galβ1-4GlacNAc | Gal α 1-3Galβ1-4GlacNAc-R |
| Galili,Gala-3′LacNAc | Galα1-3Galβ1-4GlcNAcβ-R |
| Gala | Gal α 1,4Galβ-R |
| GalCer: | Galβ1-R |
| GalNAc-GD1a: | GalNAcβ1,4Galβ1,3GalNAcβ1,4(Neu5Acα2,3)Galβ1,4Glcβ1-R |
| (IV3Neu5AcII3Neu5AcGg5Cer) | |
| Ganglio | Galβ1,3GalNAcβ1,4Galβ1,4Glcβ-R |
| GbOse3Cer: | Galα1,4Galβ1,4Glcβ1-R |
| (Gb3Cer) | |
| GbOse4Cer | GalNAcβ1,3Galα1,4Galβ1,4Glcβ1-R |
| GD1a: | Neu5Acα2,3Galβ1,3GalNAcβ1,4(Neu5Acα2,3)Galβ1,4Glcβ1-R |
| (IV3Neu5AcII3Neu5AcGg4Cer) | |
| GD1b: | Galβ1,3GalNAcβ1,4(Neu5Acα2,8Neu5Acα2,3)Galβ1,4Glcβ1-R |
| (II3(Neu5Ac)2Gg4Cer) | |
| GD1b-lactone: | II3[Neu5Ac-(2-8,1-9)-Neu5Ac]Gg4-R |
| GD1c: | Neu5Acα2,8Neu5Acα2,3Galβ1,3GalNAcβ1,4Galβ1,4Glcβ1-R |
| (IV3(Neu5Ac)2Gg4Cer) | |
| GD1α: | Neu5Ac α2,3Galβ1,3(Neu5Acα2,6)GalNAcβ1,4Galβ1,4Glcβ1-R |
| (IV3Neu5AcIII6Neu5AcGg4Cer) | |
| GD2: | GalNAcβ1,4(Neu5Acα2,8Neu5Acα2,3)Galβ1,4Glcβ1-R |
| (II3(Neu5Ac)2Gg3Cer) | |
| GD3: | Neu5Acα2,8Neu5Acα2,3Galβ1,4Glcβ1-R |
| (II3(Neu5Ac)2LacCer) | |
| GGal | Neu5Ac(α 2-3)DGalp(β1-1)-R |
| GlcCer: | Glcβ1-R |
| Globo | GalNAcβ1,3Gala1,4Galβ1,4Glcβ-R |
| Globo-H | Fuc α 2Galβ3GalNAcβ3Gal α 4Galβ4Glcβ1-R |
| Gb5 | Gal(β1-3)GalNAc(β 1-3)Gal(a 1-4)Gal(β 1-4)Glc-R |
| Gb5 | Galβ3GalNAcβ3Galα4Galβ4Glcβ1-R |
| monosialyl-Gb5 | SAα3Galβ3GalNAcβ3Galα4Galβ4Glcβ1-R |
| disialyl-Gb5 | SAα3Galβ3GalNAcβ3(SAa2-3)Galα4Galβ4Glcβ1-R |
| iso-Gb3 | Galα3Galβ4Glcβ1-R |
| iso-Gb4 | GalNAcβ3Galα3Galβ4Glcβ1-R |
| Forssman | GalNAcα3GalNAcβ3Galα4Galβ4Glcβ1-R |
| GM1a: r | Galβ1,3GalNAcβ1,4(Neu5Acα2,3)Galβ1,4Glcβ1-R |
| (II3Neu5AcGg4Cer) | |
| GM1b: | Neu5Acα2,3Galβ1,3GalNAcβ1,4Galβ1,4Glcβ1-R |
| (IV3Neu5AcGg4Cer) | |
| GM2: | GalNAcβ1,4(Neu5Acα2,3)Galβ1,4Glcβ1-R |
| (II3Neu5AcGg3Cer) | |
| GM2b | Neu5Ac(α 2-8)Neu5Ac(α 2-3)DGalp(β1-4)DGlcp(β1-1)-R |
| GM3: | Neu5Acα2,3Galβ1,4Glcβ1-R |
| (II3Neu5AcLacCer) | |
| GM4: | Neu5Acα2-3Galβ1-R |
| (I3Neu5AcαGalCer) | |
| GP1c: | Neu5Acα2,8Neu5Acα2,3Galβ1,3GalNAcβ1,4 |
| (IV3(Neu5Ac)2II3 | (Neu5Acα2,8Neu5Acα2,8Neu5Acα2,3)Galβ1,4Glcβ1-R |
| (Neu5Ac)3Gg4Cer) | |
| GP1cα: | Neu5Ac |
| (IV3Neu5AcIII6Neu5Ac,II3(Neu5Ac)3Gg4Cer) | α2,3Galβ1,3(Neu5Acα2,6)GalNAcβ1,4(Neu5Acα2,8Neu5Acα2,8Neu5Acα2,3)Galβ1,4Glcβ1-R |
| GQ1b: | Neu5Acα2,8Neu5Acα2,3Galβ1,3GalNAcβ1,4(Neu5Acα2,8Neu5Acα2,3)Galβ1,4Glcβ1-R |
| (IV3(Neu5Ac)2II3 | |
| (Neu5Ac)2Gg4Cer) | |
| GQ1bα: | Neu5Ac |
| (IV3(Neu5Ac)2III6(Neu5Ac)2Gg4Cer) | α2,3Galβ1,3(Neu5Acα2,6)GalNAcβ1,4(Neu5Acα2,8Neu5Acα2,3)Galβ1,4Glcβ1-R |
| GQ1c: | Neu5Acα2,3Galβ1,3GalNAcβ1,4(Neu5Acα2,8Neu5Acα2,8Neu5Acα2,3)Galβ1,4Glcβ1-R |
| (IV3Neu5AcII3(Neu5Ac)3Gg4Cer) | |
| GT1,GT1b | Neu5Ac(α 2-3)DGalp(β1-3)DGalNAc(β1-4)[Neu5Ac(α 2-8)Neu5Ac(α |
| | 2-3)]DGalp(β1-4)DGlcp(β1-1)-R |
| GT1a: | Neu5Acα2,8Neu5Acα2,3Galβ1,3GalNAcβ1,4(Neu5Acα2,3)Galβ1,4Glcβ1-R |
| N(V3(Neu5Ac)2II3Neu5AcGg4Cer) | |
| GT1b: | Neu5Acα2,3Galβ1,3GalNAcβ1,4(Neu5Acα2,8Neu5Acα2,3) Galβ1,4Glcβ1-R |
| (IV3Neu5AcII3(Neu5Ac)2Gg4Cer) | |
| GT1c: | Galβ1,3GalNAcβ1,4(Neu5Acα2,8Neu5Acα2,8Neu5Acα2,3)Galβ1,4Glcβ1-R |
| (II3(Neu5Ac)3Gg4Cer) | |
| GT1a: | Neu5Ac α2,3Galβ1,3(Neu5Acα2,6)GalNAcβ1,4(Neu5Acα2,3)Galβ1,4Glcβ1-R |
| (IV3Neu5AcIII6(Neu5Ac)2Gg4Cer) | |
| GT2: | GalNAcβ1,4(Neu5Acα2,8Neu5Acα2,8Neu5Acα2,3)Galβ1,4Glcβ1-R |
| GT3: | Neu5Acα2,8Neu5Acα2,8Neu5Acα2,3Galβ1,4Glcβ1-R |
| (II3(NeuAc)3LacCer) | |
| Internal Lewis x | Gal(b1-4)GlcNAc(b1-3)Gal(b1-4)[Fuc(a1-3)]GlcNAc(b1-3)Gal(b1-4)GlcNAc(b1- |
| | 3)Gal(b1-4)GlcNAc(b1-3)Gal(b1-4)Glc(b1-1)-R |
| Isoglobo | GalNAcβ1,3Gala1,3Galβ1,4Glcβ-R |
| LacCer: | Galβ1,4Glcβ1-R |
| Lacto | Galβ1,3GlcNAcβ1,3Galβ1,4Glcβ-R |
| Lewis a, | Gal(β1-3)[Fuc(α 1-4)]GlcNAc(β1-)-R |
| Lewis b, | Fuc(α 1-2)Gal(β1-3)[Fuc(α 1-4)]GlcNAc(β1-)-R |
| Lewis x,H, | Gal(β1-4)[Fuc(α 1-3)]GlcNAc(β1-)-R |
| Lewis y, | Fuc(α 1-2)Gal(β1-4)[Fuc(α 1-3)]GlcNAc(β1-)-R |
| Mollu | Fuc α 1,4GlcNAcβ1,2Man α 1,3Manβ 1,4Glcβ-R |
| Muco | Galβ1,3Galβ1,4Galβ1,4Glcβ-R |
| N-Acetyl GD3 | Neu5Ac(α 2-8)Neu5Ac(α 2-3)Gal(b1-4)Glc(b1-1)-R |
| Neogala | Galβ 1,6Galβ 1,6Galβ-R |
| Neolacto | Galβ1,4GlcNAcβ1,3Galβ1,4Glcβ-R |
| Neu5Ac(a2- | Neu5Ac(α 2-3)Gal(β1-)-R |
| 3)Gal(b1) | |
| Neu5Ac(a2- | Neu5Ac(α 2-3)Gal(β1-3)GalNAc-R |
| 3)Gal(b1- | |
| 3)GalNAc | |
| Neu5Ac(a2- | Neu5Ac(α 2-8)Neu5Ac(α 2-3)Gal-R |
| 8)Neu5Ac(a2- | |
| 3)Gal | |
| (Sia)₃ | Neu5Acα2-8Neu5Acα2-8Neu5Acα-R |
| 3′-SL | Neu5Acα2-3Galβ1-4Glcβ-R |
| N-Glycolyl GM3 | Neu5Gc(α 2-3)Gal(β1-4)Glc(β1-1)-R |
| NOR1 | Gal(α 1-4)GalNAc(β 1-3)Gal(α a 1-4)Gal(β 1-4)Glc-R |
| NOR2 | Gal(α 1-4)GalNAc-(β 1-3)Gal(α 1-4)GalNAc(β 1-3)Gal(α 1-4)Gal(β 1-4)Glc-R |
| NOT int | GalNAc(β 1-3)Gal(α 1-4)GalNAc(β1-3)Gal-(α 1-4)Gal(β 1-4)Glc-R |
| O antigen | Fuc (α 1-2)Gal(β1-3)GlcNAc(β1-3)Gal-R, |
| OAc-GT1b | Neu5Ac(α 2-3)DGalp(β1-3)DGalNAc(β1-4)XNeu5Ac9Ac(α 2-8)Neu5Ac(α 2- |
| | 3)]DGalp(β-4)DGlcp(β1-1)-R |
| P antigen (Gb4) | Gal(α 1-4)Gal-(β 1-4)GlcNAc(β 1-3)Gal(β 1-4)Glc-R |
| Pk Antigen (Gb3) | Gal(α 1-4)Gal(β1-4)Glc-R |
| Pi | Galα1-4Galβ1-4GlcNAcβ-R |
| Schisto | GalNAcβ1,4Glcβ-R |
| Sialyl Lewis a, | Neu5Ac(α 2-3)Gal(β1-3)[Fuc(α 1-4)]GlcNAc(β1-)-R |
| Sialyl Lewis c | Neu5Ac(α 2-3)Gal(β1-3)GlcNAc(β1-)-R |
| Sialyl Lewis x, | Neu5Ac(α 2-3)Gal(β1-4)[Fuc(α 1-3)]GlcNAc(β1-3)Gal(β1-4)GlcNAc(β1-3)Gal(β1-)-R |
| Sialyl Lewis x, | Neu5Ac(α 2-3)Gal(β1-4)[Fuc(α 1-3)]GlcNAc(β1-)-R |
| Sialyl Lewis x-i | Neu5Ac(α 2-3)Gal(β1-4)[Fuc(α 1-3)]GlcNAc(β1-3)Gal(β1-4)GlcNAc(β1-3)Gal(β1-)-R |
| LNT | Galβ1-3GlcNAcβ1-3Galβ1-4Glcβ-R |
| LNnT | Galβ1-4GlcNAcβ1-3Galβ1-4Glcβ-R |
| Sialyl-TF | Neu5Ac(α 2-6) Gal (β1-3) α GalNAc-R, |
| sialyl-Tn | Neu5Ac(α 2-6)GalNAc-R, |
| sLac | NeuAc-Gal0-3GicNAcB-3GalB-4Glc-R |
| Spirometo | Galβ1,4Glcβ1,3Galβ-R |
| Sulfatide: | Sulfate3Galβ1-R |
| TF/Core-1 | α Gal (β1-3)aGalNAc-R, |
| 3′-sialyl-TF | Neu5Acα2-3Galβ1-3GalNAcα-R |
| Tn | α GalNAc-R |
| 6-SiaβTF | Neu5Acβ2-6(Galβ1-3)GalNAcα-R |
| 3-LacNAc-Tₙ | Galβ1-4GlcNAβ1-3GalNAcα-R |
| 6-LacNAc-Tₙ | Galβ1-4GlcNAβ1-6GalNAcα-R |
| 6′SLN | Neu5Acα2-6Galβ1-4GlcNAcβ-R |
| Core 2 | GlcNAcβ1-6(Galβ1-3)GalNAcα-R |
| Core 4 | GlcNAcβ1-3(GlcNAcβ1-6)GalNAcα-R |
| Trifucosyl-Lewis b Antigen | Fuc(α 1-2)Gal(b1-3)[Fuc(α 1-4)]GlcNAc(b1-3)Gal(b1-3)[Fuc(α 1-4)]GlcNAc(b1-)-R |
| Trifucosyl-Lewis y Antigen | Fuc(α 1-2)Gal(b1-4)[Fuc(α 1-3)]GlcNAc(b1-3)Gal(b1-4)[Fuc(α 1-3)]GlcNAc(b1-)-R |
| Type 1 | (GalNAc(α 1-3)[Fuc(α 1-2)]Gal(β1-3)GlcNAc[Fuc(α 1-4)]-R), |
| Type 1 A | GalNAc(α1-3)[Fuc(α 1-2)]Gal(β1-3)GlcNAc(β1-3)Gal-R, |
| Type 2 A | GalNAc(α1-3)[Fuc(α 1-2)]Gal(β1-4)GlcNAc-R, |
| Type 3 A | GalNAc(α1-3)[Fuc(α 1-2)]Gal(β1-3)GalNAc(β1-3)Gal[Fucα 1-2)-R, |
| Type 4 A | GalNAc(α1-3)[Fuc(α 1-2)]Gal(β1-3)GalNAc(β1-3)Gal(a 1-4)Gal(β 1-4)Glc--R, |
| VIM-2 | Neu5Ac(α 2-3)Gal(β1-4)GlcNAc(β1-3)Gal(β1-4)[Fuc(α-3)]GlcNAc(β1-)-R |
| Type 4 A | GalNAc(α1-3)[Fuc(α 1-2)]Gal(β1-3)GalNAc(β1-3)Gal(a 1-4)Gal(β 1-4)Glc--R, |
| Man₃ | Manα1-3(Manα1-6)Manα-? |
| 3′SLN | Neu5Acα2-3Galβ1-4GlcNAcβ-R |
| 6′SL | Neu5Acα2-6Galβ1-4Glcβ-R |
| Human milk oligosaccharide | see WO 2012/092153, WO 2010/120682, WO 2005/055944, U.S. Pat. No. 5,945,314 |

wherein R may be one or more of the following: a carbohydrate/s, a peptide/s, a lipid/s, a linker/s and a chemical compound/s or substance/s or molecule/s; or R comprises one or more of the following: a carbohydrate/s, a peptide/s, a lipid/s, a linker/s or a chemical compound/s or substance/s or molecule/s;
viii) any one of lipid carriers of i)-vii) further coupled to TF disaccharide, Core-1 structure, Tn monosaccharide, Sialyl-TF mono- or disialylated, Sialyl-Tn, Polysialic acid, or mannose-6-phosphate moiety;
ix) any one of lipid carriers of i)-viii) further coupled to N-Glycan or O-glycan moiety;
x) any one of lipid carriers of i)-ix) further coupled to a carbohydrate moiety;
xi) a truncated or elongated derivative of any one of lipid carriers of i)-x);
xii) a phosphorylated, sulfated or acetylated derivative of any one of lipid carriers of i)-xi);
xiii) derivatives and analogs of any of (i)-(xii).

The present invention may also comprise said modified microorganism as defined elsewhere herein, wherein said microorganism is naturally-occurring, preferably said naturally-occurring microorganism is obtainable from one or more of the following sources:
i) microflora or microbiota of an animal, preferably a vertebral organism, preferably microbiota or microflora of a digestive or urogenital system or skin microflora of said vertebral organism; further preferably said naturally-occurring microorganism is obtainable from the gut, feces, oral or nasal cavity, vagina, lung, sputum, other mucus sources or urine of said vertebral organism; most preferably said mammalian organism is human; and
ii) soil microflora or microbiota;
iii) microbiota from plants, preferably leaves, fruits or berries, or marine ecosystems.
iv) microbiota from food.

The present invention may also envisage said modified microorganism as defined elsewhere herein, wherein said microorganism is a gram-positive bacterium, a gram-negative bacterium, a fungus or protozoa.

The present invention may also comprise said modified microorganism as defined elsewhere herein, wherein said microorganism is one or more of the following:
i) a bacterium; preferably said bacterium is gram-positive or gram-negative bacterium; non-pathogenic and/or opportunistic pathogen; further preferably said bacterium is a gram-positive bacterium; further preferably said gram-positive bacterium is selected from the genera consisting of: Lactobacillus, Bifidobacterium, Clostridium, Enterococcus, Pediococcus and Streptococcus; most preferably said gram-positive bacterium is selected from the group consisting of: Lactobacillus paracasei, Lactobacillus reuteri; and
ii) a fungus; preferably said fungus is selected from the group consisting of: Candida yeasts, Saccharomyces yeasts and yeasts in the family Dipodascaceae; further preferably said Dipodascaceae yeasts are Galactomyces, Geotrichum or Saprochaete yeasts, most preferably said Saccharomyces yeast is Saccharomyces boulardii, S. cerevisiae, S. pastorianus, or Schizosaccharomyces pombe.

In a second aspect, the present invention relates to a composition comprising one or more of the modified microorganisms as defined elsewhere herein and preferably said composition comprises a mixture of same or different modified microorganisms as defined elsewhere herein

Preferably, said composition is a pharmaceutical, diagnostic, probiotic or prebiotic composition.

In a third aspect, the present invention relates to a vaccine or adjuvant comprising the microorganism or said composition as described herein.

In a fourth aspect, the present invention relates to a method for producing or isolating a modified microorganism as defined elsewhere herein comprising
i) a cell and
ii) a heterologous lipid carrier, said lipid carrier comprising:
   a) a lipid portion, wherein said lipid portion is at least partially associated with an exterior surface of said cell of said modified microorganism, wherein said lipid portion comprises a ceramide-like glycolipid moiety and/or a fatty acid moiety; preferably said exterior surface of said cell comprising: a cell wall and/or a cell membrane and/or an outer cell membrane and/or a polysaccharide; further preferably said lipid portion is at least partially incorporated and/or adhered and/or bound to said exterior surface of said cell; and
   b) a non-lipid portion, wherein said microorganism is capable of locating and/or displaying said non-lipid portion or fragment thereof onto the exterior surface of said cell, preferably said non-lipid portion comprising a carbohydrate moiety, lipopeptide moiety, a linker, a chemical compound, or a peptide moiety;
iii) optionally, said modified microorganism further comprising a heterologous steroid moiety; preferably said steroid moiety is cholesterol, a derivative or analog thereof;
said method comprising:
i′) culturing:
   a′) a microorganism or mixture of microorganisms, preferably said microorganism or mixture of microorganisms are naturally-occurring; further preferably said naturally-occurring is obtainable from one or more of the following sources:
      aa′) microflora or microbiota of an animal, preferably a vertebral organism, preferably microbiota or microflora of a digestive or urogenital system or skin microflora of said vertebral organism; further preferably said naturally-occurring microorganism is obtainable from the gut, feces, oral or nasal cavity, vagina, lung, sputum, other mucus sources or urine of said vertebral organism; most preferably said mammalian organism is human; and microflora of a vertebral organism, preferably microflora of a digestive or urinary system or skin microflora of said vertebral organism; further preferably said naturally-occurring microorganism is obtainable from feces or urine of said vertebral organism; most preferably said mammalian organism is human;
      bb′) soil microflora or microbiota;
      cc′) microbiota of plants, preferably leaves, fruits or berries, or marine ecosystems;
      dd′) microbiota from food;
   in growth medium, e.g. BSM (Bifidobacterium Selective Medium), LB (Lysogeny broth), MRS (de Man, Rogosa and Sharpe Medium), WC (Wilkins and Chalgren Medium), SBSM or ABM;
   optionally, isolating and/or enriching said microorganism or mixture of microorganisms from said one or more sources;
ii′) adding a loading medium to said microorganism or mixture of microorganisms, preferably said loading medium is a culture medium, e.g. BSM (Bifidobacterium Selective Medium), LB (Lysogeny broth), MRS (de Man, Rogosa and Sharpe Medium), PBS, WC (Wilkins and Chalgren Medium) SBSM or ABM; further preferably said loading medium is a buffer solution, most preferably said adding is carried out under growth conditions; further most preferably said adding is the resuspending of said microorganism or mixture of microorganisms in said loading medium;
   iii′) adding said solubilized heterologous lipid carrier to the suspension of said microorganism or mixture of microorganisms in said loading medium; preferably said adding is carried out at a temperature in the range between about 4° C. and about 70° C., preferably between about 12 and about 65° C., further preferably between about 18° C. and about 50° C., further preferable between about 23° C. and about 46° C., further preferably in the range from about 30° C. to about 55° C.; further preferably said temperature is selected from the group consisting of: 30, 37, 46 and 55° C.;
iv′) optionally, isolating said microorganism or mixture of microorganisms comprising said lipid carrier, preferably by the means of affinity purification (e.g. with one or more toxins, e.g. CT, SW/2 and/or LT, pathogen receptor(s) or parts thereof).

The present invention may also comprise said method as defined elsewhere herein, further comprising
v′) culturing said microorganism in a culture medium under growth conditions and isolating said microorganism from said culture medium;
vi′) optionally, discarding the microorganism or mixture of microorganisms not comprising said lipid carrier (e.g. after isolating said microorganism or mixture of microorganisms comprising said lipid carrier);
vii′) optionally, identifying said isolated microorganism, preferably by the means of mass spectroscopy and/or nucleic acid sequencing and/or microbiological analysis and/or biochemical analysis;
viii′) optionally, carrying out said method under different condition (preferably with another growth medium and/or loading medium and/or under different loading conditions (e.g. of the lipid carrier to the microorganism) and/or growing conditions (e.g. temperature and/or pH) and evaluating the effect of said condition on the association (e.g. strength, stability, concentration, amount of) of said lipid carrier with said microorganism.

The present invention may also comprise said method as defined elsewhere herein, wherein step ii′) is preceded by bringing said microorganisms into contact with trichloroacetic acid, a detergent, or an antibiotic, preferably with trichloroacetic acid.

The present invention may also envisage said method as defined elsewhere herein, wherein said antibiotic inhibits cell wall synthesis or disturbs membrane structure.

The present invention may also envisage said method as defined elsewhere herein, wherein said loading and/or growth medium comprises cholesterol or analog or derivative thereof.

The present invention may also encompass said method as defined elsewhere herein, wherein said growth medium and/or loading medium comprises a lipase or an inhibitor of lipid synthesis.

The present invention may also comprise said method as defined elsewhere herein, wherein said culturing is carried out under anaerobic conditions.

The present invention may also envisage said method as defined elsewhere herein, wherein said culture medium is not suitable for an optimal growth of said microorganism, preferably said culture medium is not suitable for supporting a highest growth rate and/or shortest generation time for said microorganism.

The present invention may also encompass said method as defined elsewhere herein, wherein said culture medium comprises propionic acid.

The present invention may also encompass said method as defined elsewhere herein, wherein said growth conditions are not suitable for an optimal growth of said microorganism, preferably said growth conditions are not suitable for supporting a highest growth rate and/or shortest generation time for said microorganism.

Preferably, in the method for producing or isolating a modified microorganism said growth conditions comprise one or more of the following:
i) a temperature in the range between about 4° C. and about 70° C., preferably between about 12° C. and about 60° C., further preferably between about 18° C. and about 50° C., further preferable between about 23° C. and about 46° C., further preferably in the range from about 30° C. to about 55° C.; most preferably said temperature is selected from the group consisting of: about 30, 37, 46 and 55° C.
ii) a pH in the range between about 1 and about 10, preferably between about 3 and about 9, further preferably between about 4 and about 8, further preferably in the range from about 3 to about 7, most preferably pH in the range from about 5 to about 6.

Also comprised by the present invention is said method as defined elsewhere herein, wherein prior to adding said solubilized heterologous lipid carrier the suspension of said microorganism is having an optical density (OD) in the range between about OD 0.1 and about OD 100, preferably between about OD 0.3 and about OD 30, preferably between about OD 0.5 and about OD 10, further preferably between about OD 1 and about OD5, most preferably in the range from about OD 1 to about OD 2, further most preferably said OD may be measured at a wavelength in the range from about 540 to about 660 nm, further most preferably OD may be measured at a wavelength of about 600 nm.

The present invention may also encompass said method as defined elsewhere herein, wherein said adding of the solubilized heterologous lipid carrier is carried out at a temperature in the range between about 4° C. and about 70° C., preferably between about 12° C. and about 60° C., further preferably between about 18° C. and about 50° C., further preferably between about 23° C. and about 46° C., further preferably in the range from about 30° C. to about 55° C.; most preferably said temperature is selected from the group consisting of: about 30, 37, 46 and 55° C.

The present invention may also encompass said method as defined elsewhere herein, wherein the specific combination of said microorganism, culture medium (i.e., growth medium), growth temperature, temperature of adding of said solubilized heterologous lipid carrier (i.e., loading temperature), pH and loading medium are selected from the group consisting of the following combinations as shown in Table 1.

The present invention may also comprise said method as defined elsewhere herein, further comprising determining whether the association of the heterologous lipid carrier with an exterior surface of said cell of said modified microorganism resists a treatment with 0.3% bile salts, optionally in combination with pancreatine juice in PBS or DPBS for at least 1 hour at least 37° C.

The present invention may also comprise said method as defined elsewhere herein, wherein resistance means that at least 80% of the heterologous lipid carrier associated with an exterior surface of said cell of said modified microorganism remains associated after a treatment with 0.3% bile salts, optionally in combination with pancreatine juice in PBS or DPBS for at least 1 hour at least 37° C.

In a fifth aspect, the present invention relates to a modified microorganism comprising
i) a cell and
ii) a heterologous lipid carrier, said lipid carrier comprising:
   a) a lipid portion, wherein said lipid portion is at least partially associated with an exterior surface of said cell of said modified microorganism, wherein said lipid portion comprises a ceramide-like glycolipid moiety and/or a fatty acid moiety; preferably said exterior surface of said cell comprising: a cell wall and/or a cell membrane and/or an outer cell membrane and/or a polysaccharide; further preferably said lipid portion is at least partially incorporated and/or adhered and/or bound to said exterior surface of said cell; and
   b) a non-lipid portion, wherein said microorganism is capable of locating and/or displaying said non-lipid portion or fragment thereof onto the exterior surface of said cell, preferably said non-lipid portion comprising a carbohydrate moiety, a lipopeptide moiety, a linker, a chemical compound, or a peptide moiety;
iii) optionally, said modified microorganism further comprises a heterologous steroid moiety; preferably said steroid moiety is cholesterol, a derivative or analog thereof;
   wherein said modified microorganism is obtainable or obtained by the method for producing or isolating as defined elsewhere herein,
   wherein said cell of said modified microorganism does not comprise a mycomembrane, preferably said mycomembrane is located in the exterior surface of said cell of said modified microorganism, and wherein said non-lipid portion is not alpha-galactosylceramide.

In a sixth aspect, the present invention relates to a method for screening for a lipid carrier, growth medium, loading medium, loading conditions or growth conditions, said method comprises
i) providing the microorganism, composition, vaccine or adjuvant as defined elsewhere herein;
ii) providing a lipid carrier;
iii) exposing (e.g. adding or loading) said lipid carrier to said microorganism, composition, vaccine or adjuvant as defined elsewhere hereinunder suitable conditions;
iv) optionally, evaluating the effect of the lipid portion (e.g. a fatty acid) and/or the non-lipid portion (e.g. polypeptide) of said lipid carrier on association (e.g. stability, strength, amount and/or concentration) between said lipid carrier with said microorganism, composition, vaccine or adjuvant as defined elsewhere herein;
v) optionally, carrying out said method under different condition, preferably with another growth medium and/or loading medium and/or under different loading conditions (e.g. of the lipid carrier to the microorganism) and/or growing conditions (e.g. temperature and/or pH) (e.g. different from those as described elsewhere herein) and evaluating the effect of said condition (preferably another growth medium and/or loading medium and/or under different loading conditions (e.g. temperature and/or pH) on the association e.g. strength, stability, concentration and/or amount) of said lipid carrier with said microorganism.

### BRIEF DESCRIPTION OF THE FIGURES

FIG. 1: Table illustrating the loading stability of different lipid carriers. The herein described lipid carriers are shown in comparison to the loading stability of the same lipid carriers according to a different protocol. New culture conditions and/or a new pre-treatment allowed or increased the stability. (*=Cholesterol was added to increase the loading stability; n.d.=not determined)

FIG. 2: Effect of Treatments on the stability of the association of Gb3 with isolate Lactobacillus paracasei L3.

The bacteria (Lactobacillus paracasei L3) were treated as depicted in the figure, thoroughly washed before being incubated overnight in BSM at 37° C. under anaerobic conditions with 3 μg/ml of Gb3 and 1% cholesterol.

To assess the stability of the association, loaded cells were incubated with Ileum juice containing 0.3% bile salts at 37° C. for one hour (right bars). Control cells were incubated in PBS at 37° C. for one hour (left bars). The presence of the receptor on the surface of the bacteria was analyzed by ELISA using Alkaline-Phosphate labelled Shiga Toxin 1 (AP-Stx1 toxin).

FIG. 3: Effect of cholesterol on association between FA-peptide and isolate Lactobacillus paracasei L3.

The bacteria (Lactobacillus paracasei L3) were incubated overnight in SBSM at 37° C. under anaerobic conditions with 10 μg/ml of FA-Peptide 1 or FA-Peptide 2 or GM1 with or without 1% cholesterol. The presence of GM1 or the Peptide on the surface of the isolates was analyzed by ELISA using AP-cholera toxin.

FIG. 4: Effect of TCA pre-treatment on the stability of the association between FA-peptide and isolate Lactobacillus paracasei L3.

The bacteria (Lactobacillus paracasei L3) were treated or not with 10% TCA at 90° C. for 15 minutes before being washed and incubated overnight in SBSM at 37° C. under anaerobic conditions with 10 μg/ml of FA-Peptide 1 with 1% cholesterol. The presence of the Peptide on the surface of the isolates was analyzed by ELISA using AP-cholera toxin.

To assess the stability of the association, loaded cells were incubated with Ileum juice containing 0.3% bile salts at 37° C. for one hour. Control cells were incubated in PBS at 37° C. for one hour ((−) Gut Juice).

FIG. 5: Lactobacillus paracasei L3-GM1-strain inhibits binding to GM1. A) CTB binding to coated GM1. B) CTB binding to coated GM1 in the presence of L3-GM1 (2.5 mg/ml−dry weight).

FIG. 6: In vivo results of experiment 1.

Timeline and graph for the control groups, not infected and infected with V. cholerae (>10 times the LD50).

FIG. 7: In vivo results of experiment 1.

Timeline and graph for two mouse groups infected with V. cholerae (>10 times the LD50). One control group with unloaded L3 and one with GM1-loaded L3 (L3-GM1). At each application time, mice received 2.5 mg (dry weight) of the strain.

FIG. 8: In vivo results of experiment 2.

Timeline and graph for the control groups, not infected and infected with V. cholerae (>30 times the LD50).

FIG. 9: In vivo results of experiment 2.

Timeline and graph for two mouse groups infected with V. cholerae (>30 times the LD50). One control group with unloaded L3 and one with GM1-loaded L3 (L3-GM1). At each application time, mice received 0.5 mg (dry weight) of the strain.

FIG. 10:

The bacteria (Lactobacillus paracasei L3) were incubated overnight in SBSM at 37° C. under anaerobic conditions with 1 μg/ml GM1 or 3 μg/ml Gb3. The cells were washed, and the pellet resuspended in buffer or Ileum juice containing 0.3% bile salts and incubated at 37° C. for one hour. The presence of GM1 or Gb3 on the surface of the isolates was analyzed by ELISA using AP-cholera toxin and AP-Stx2 toxin, respectively.

FIG. 11: Binding of HRP-Cholera toxin to isolates L9, L3 and Lac9.

Isolates were incubated overnight in PBS at 30° C. with or without GM1 at 5 μg/ml. The isolates were extensively washed. The presence of GM1 on the surface of the isolates was analyzed by ELISA using HRP-cholera toxin. As used herein, “L9”=Lactobacillus reuteri, “L3”=Lactobacillus paracasei, “Lac9”=Lactobacillus reuteri.

FIG. 12: Stability of the association of GM1 with L3 isolate.
A) Binding of labeled cholera toxin to the GM1-loaded strain L3 after pasteurization (15 min 70° C.). B) Binding of labeled cholera toxin to the GM1-loaded strain L3 after incubation at pH=1 for 1 hour at 37° C. C) Binding of labeled cholera toxin to the GM1-loaded strain L3 after freezing and thawing. D) Binding of labeled cholera toxin to the GM1-loaded strain L3 after freezing drying and re-hydration. E) Binding of labeled cholera toxin to the GM1-loaded strain L3 after 4 hours incubation at 37° C. F) Binding of labeled cholera toxin to the GM1-loaded strain L3 after incubation in gastric juice for 30 min at 37° C.

### DETAILED DESCRIPTION OF THE INVENTION

The following detailed description refers to the accompanying Examples and Figures that show, by way of illustration, specific details and embodiments, in which the invention may be practiced. These embodiments are described in sufficient detail to enable those skilled in the art to practice the invention. Other embodiments may be utilized such that structural, logical, and eclectic changes may be made without departing from the scope of the invention. Various aspects of the present invention described herein are not necessarily mutually exclusive, as aspects of the present invention can be combined with one or more other aspects to form new embodiments of the present invention.

Herein is described, how different bacteria strains can be loaded with a lipid carrier. Such a lipid carrier may comprise or consist of a toxin receptor. The inventors were after massive research able to develop a loading protocol ensuring the stable loading of the bacteria, thus developing ‘armed’ bacteria for example for toxin clearance in a living organism.

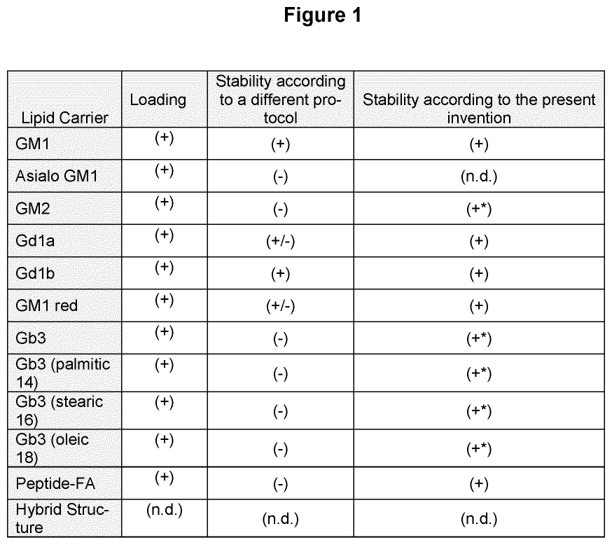

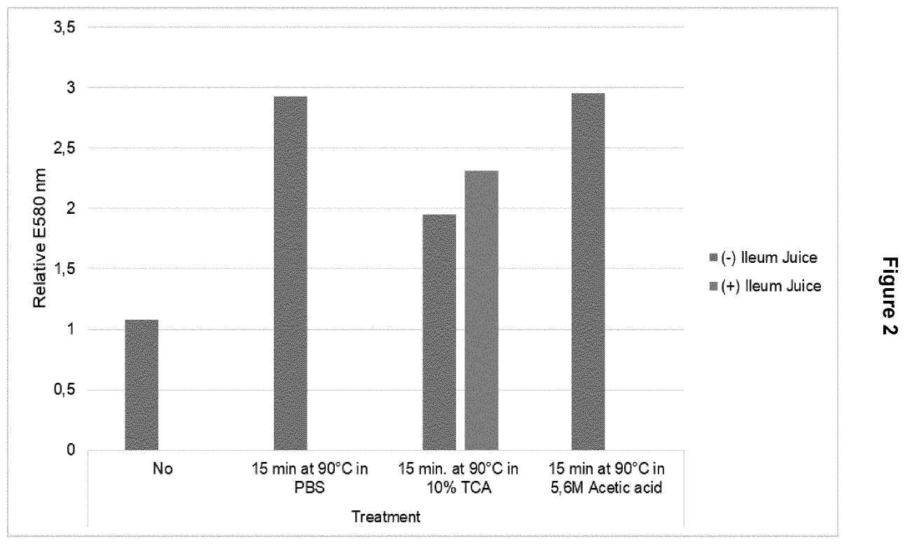

Culturing and loading under the conditions summarized in Table 2 and 3 allowed an association of different lipid carriers that resisted the stability test. Meaning Lactobacillus bacteria could for example be loaded successfully with the lipid carriers GM1, Gd1a, Gd1b, GM1red, Gb3, Gb3 (palmitic 14), Gb3 (steric 16), Gb3 (oleic 18), and peptides were resisting a stability test (FIGS. 1, and 2 to 4).

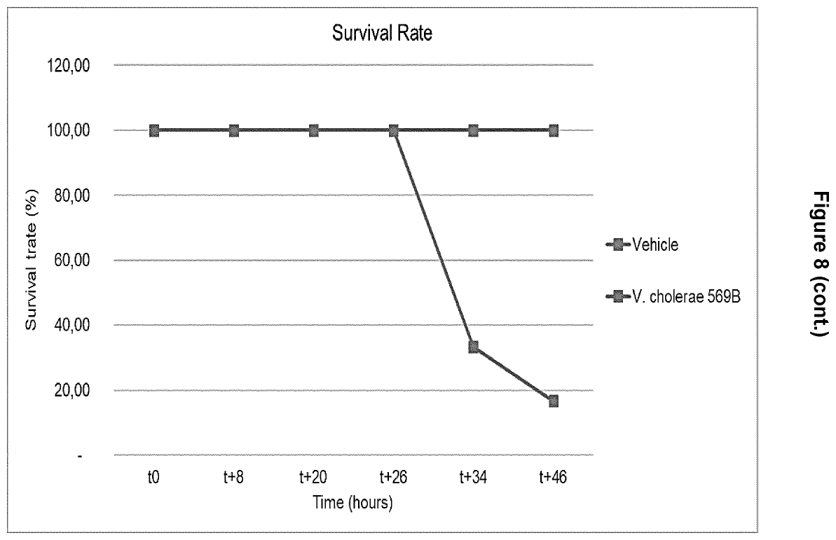

Furthermore, mice treated with over 10 times the LD50 dose of V. cholerae and a loaded bacteria strain binding cholera toxin survived an experiment, while animals infected with V. cholerae not receiving a loaded bacteria strain died within the experiment (FIG. 7). This proved the stability of the loaded bacterium in vivo, and an effective treatment. Even mice injected with V. cholerae exceeding 30 times the LD50 dose, still presented a beneficial effect, in increasing the survival time of the infected animals (FIG. 9).

### Definitions

Unless otherwise specified, the terms used herein have their common general meaning as known in the art (e.g. as defined by IUPAC. Compendium of Chemical Terminology, 2nd ed. (the “Gold Book”, compiled by A. D. McNaught and A. Wilkinson. Blackwell Scientific Publications, Oxford (1997); XML on-line corrected version: http://goldbook.iupac.org (2006-) created by M. Nic, J. Jirat, B. Kosata; updates compiled by A. Jenkins. ISBN 0-9678550-9-8. https://doi.org/10.1351/goldbook).

As used herein, the term “microorganism” may refer to any organism too small to be viewed by the unaided eye, e.g. bacteria, virus, protozoa, archaea, fungi and algae. Unless defined otherwise, the term “microorganism(s)” used herein includes bacteria, viruses (e.g., Norovirus, Herpes simplex virus 1 and 2 (HSV-1 and HSV-2), influenza virus, respiratory syncytial virus Human papilloma virus (HPV) etc.), fungi (including unicellular and filamentous fungi), yeasts, and protozoa and multi-cellular parasites. Typical sources of microorganisms described herein include feces, gut, skin, nose, ear, mouth, eye, urogenital or respiratory tract, breast milk, foods (including but not limited to: milk products, meet, etc.), pure cultures, soil, water and plants.

Exemplary non-limiting microorganisms include bacteria (e.g. gram-positive or gram-negative (e.g. from genus Bacteroides, e.g. B. vulgatus, B. xylanisolvens, B. fragilis) bacteria); gram-positive bacteria include bacteria selected from the genera consisting of: Lactobacillus (e.g. Lactobacillus paracasei, Lactobacillus rahmnosus, Lactobacillus reuteri, Lactobacillus crispatus, Lactobacillus fermentum, Lactobacillus gasseri, Lactobacillus salivarius, Lactobacillus plantarum, Lactobacillus casei, Lactobacillus acidophilus or Lactobacillus vaginalis, Lactobacillus murinus, Lactobacillus brevis), Bifidobacterium (e.g. Bifidobacterium animalis, Bifidobacterium breves Bifidobacterium adolescentis, Bifidobacterium longum, Bifidobacterium infantis or Bifidobacterium pseudocatenulatum), Clostridium (e.g. Clostridium perfringens, Clostridium coccides), Enterococcus (e.g. Enterococcus avium, Enterococcus faecalis, Enterococcus faecium, Enterococcus gallinarum, Enterococcus casseliflavus or Enterococcus sp.), Pediococcus (e.g. Pediococcus pentosaceus) and Streptococcus (e.g. Streptococcus salivarius or Streptococcus vestibularis); fungi including fungi selected from the group consisting of: Candida yeasts, Saccharomyces yeasts (e.g. S. cerevisiae and S. pombe) and yeasts in the family Dipodascaceae; further preferably said Dipodascaceae yeasts are Galactomyces, Geotrichum or Saprochaete yeasts; and protozoa (e.g. Chilomastix mesnili, Endolimax nana, Entamoeba coli, Entamoeba dispar, Entamoeba hartmanni, Entamoeba polecki or Iodamoeba buetschlii). Further exemplary non-limiting gram-negative bacteria include bacteria of Enterobacteriaceae family, e.g. genera Escherichia (e.g. Escherichia sp. E. coli, E. fergusoni;), Klebsiella (e.g. K. pneumoniae, Klebsiella sp.), Enterobacter (e.g. E. cloacae, Enterobacter sp.), Proteus (e.g. P. mirabilis, Proteus sp.), Morganella (e.g. M. morganii Morganella sp.), Citrobacter (e.g. Citrobacter sp.), Serratia (e.g. Serratia sp.).

As used herein, the term “pathogenic microorganism” may refer to microorganisms, which have (i.e. express and secrete) a toxin, include microorganisms as described herein that can cause lesion and/or disease of mucosa (e.g. virus, e.g. Norovirus, Rotavirus, Herpes simplex virus 1 and 2 (HSV-1 and HSV-2), Influenza virus, Respiratory syncytial virus, Human papilloma virus (HPV) etc., protozoa (e.g. Giardia, Cryptosporidium spp. and Blastocystis spp.)), including but not limited to buccal mucosa, esophageal mucosa, gastric mucosa, intestinal mucosa, nasal mucosa, olfactory mucosa, oral mucosa, bronchial mucosa, mucosa of the respiratory tract, uterine mucosa, endometrium (mucosa of the uterus), vaginal mucosa, penile mucosa by, inter alia, a toxin. “Pathogenic microorganisms” further include microorganisms that cause lesions and/or disease of the gastrointestinal tract such as diarrhea.

However, preferably the obtained or obtainable modified microorganism is non-pathogenic. Nonpathogenic microorganisms preferably include, but are not limited to, microorganisms categorized as Generally Recognized As Safe (GRAS). Non-pathogenic microorganisms further preferably include but are not limited to lactic acid bacteria or bifidobacteria. They may also include opportunistic pathogenic microorganisms. Preferably, the non-pathogenic microorganism of the present invention may be used as an active ingredient (e.g. an active agent) in a pharmaceutical composition (e.g. it can be used live or pasteurized or otherwise inactivated). It can also be used in a composition comprising only one type of non-pathogenic microorganism or more than one microorganism, e.g. and each of such a microorganism can carry one or more type of glycolipid or lipopeptide.

According to the present invention, said modified microorganism comprising a cell and a heterologous lipid carrier as defined elsewhere herein may optionally further comprise a heterologous steroid moiety, preferably said steroid moiety is cholesterol, a derivative or analog thereof. Further, according to the present invention, said cell of said modified microorganism as described elsewhere herein in the present invention does not comprise a mycomembrane, preferably, said mycomembrane is located in the exterior surface of said cell of said modified microorganism.

In this context, a “mycomembrane (MM)” refers to a mycobacterial envelope composed of very-long chain fatty acids and mycolic acids (MA), which is known to a person skilled in the prior art. Glycolipids, phospholipids and lipoglycans, together with proteins, presumably compose the outer leaflet of the MM. The most recent model schematically divides the mycobacterial cell envelope in three entities: an outermost layer (OL), also called capsule in the case of pathogenic species, a cell wall (CW) and a conventional plasma membrane (PM).

The capsule of mycobacterial pathogens such as Mtu is mainly composed of glucan and proteins, with only a tiny amount of lipids whereas the OL of non-pathogens is primarily constituted of proteins. The CW is a giant tripartite complex composed of the MM, AG and peptidoglycan (PG), also known as the mAGP complex. The MM exhibits a non-conventional bilayer organization in which the inner leaflet is made of very long-chain MA linked to AG, which in turn is covalently attached to PG. [Chiaradia et al., (2017). Scientific Reports 7:1280].

As used herein, the term “exterior surface” includes any localization accessible from the outside of the microorganism of the present invention. Preferably, said localization may be directly exposed to the environment, i.e., be in direct contact with the environment, or may be in indirect contact, i.e., be in indirect contact with the environment, e.g. via pores (e.g. porins) that connect the periplasm with the environment. Hence, the non-lipid portion of the lipid carrier of the present invention may be associated with (e.g. present) or integrated in the inner membrane, outer membrane, cell wall, cell membrane, polysaccharide (e.g. capsule polysaccharide), pilus, flagellum or fimbria of the modified microorganism of the present invention. Further preferably the non-lipid portion of the lipid carrier of the present invention can be bound by a soluble (or non-soluble) binding molecule, receptor or toxin (e.g. in the outer membrane, cell wall, cell membrane, polysaccharide (e.g. capsule polysaccharide), pilus, flagellum or fimbria of the modified microorganism of the present invention) or be recognized by the immune system generating or blocking a specific immune response.

The terms “isolated” or “isolation” as used herein, refer to the separation of the obtained microorganism from the starting composition comprising a plurality of microorganisms, e.g. a sample of the natural environment of the microorganism or any other used source material. A further example of an isolated microorganism of the present invention may refer to a microorganism isolated through (e.g. by the means of or as part of) any screening application/s (e.g. a method and/or use) as described herein, e.g. as a positive binder/associator (e.g. from a culture of microorganisms).

As used herein, the term “heterologous” may refer to an entity (e.g. lipid carrier or chemical compound or substance or molecule) of an origin different from the cell of the microorganism of the present invention (e.g. said entity is not expressed or synthetized by said microorganism).

As used herein, the term “lipid” may refer to a substance of biological and synthetic origin that is soluble in nonpolar solvents. Exemplary non-limiting lipids of the present invention include saponifiable lipids, such as, but not limited to, glycerides (e.g. fats and oils) and phospholipids, as well as non-saponifiable lipids and steroids, ceramide-like glycolipids, fatty acids, amino alcohols (e.g. sphingosines), sphingolipids.

As used herein, the term “carrier” may refer to a diluent, adjuvant, excipient, or vehicle with which a substance is administered. Exemplary carrier in the sense of the present invention is a lipid carrier comprising: a) a lipid portion, wherein said lipid portion is at least partially associated with an exterior surface of said cell of said modified microorganism, wherein said lipid portion comprising: a ceramide-like glycolipid moiety (e.g., comprising a ceramide moiety, a sphingolipid moiety or sphingosine moiety) and/or a fatty acid moiety; preferably said exterior surface of said cell comprising: a cell wall and/or a cell membrane and/or an outer cell membrane and/or a polysaccharide (e.g. capsule polysaccharide); further preferably said lipid portion is at least partially incorporated and/or adhered and/or bound to said exterior surface of said cell, even more preferably said lipid portion further comprises an amino alcohol moiety, preferably the amino alcohol moiety is sphingosine; and b) a non-lipid portion, wherein said microorganism is capable of locating and/or displaying said non-lipid portion or fragment thereof (e.g. a peptide (e.g. an immunologically active peptide) or a protein (e.g. an anti-inflammatory cytokine), an antibody (e.g. a single domain antibody) or a fragment thereof, and other derivatives (e.g. haptens, or parts of pathogenic organisms (for vaccination)) coupled to the lipid portion) onto the exterior surface of said cell, preferably said non-lipid portion comprising a carbohydrate moiety, a lipopeptide moiety (e.g. a glycosylated lipopeptide moiety, e.g. a carbohydrate antigen) or a peptide moiety, wherein preferably said carbohydrate moiety of said non-lipid portion is a sialic acid residue; further preferably said non-lipid portion is capable of binding and/or reducing toxicity and/or neutralizing a toxin, binding and/or reducing the pathogenicity and/or neutralizing a pathogenic microorganism (e.g. a bacterium, fungus or protozoa), binding a receptor of a toxin, and binding a receptor of a pathogenic microorganism. Said heterologous lipid carrier of said modified microorganism according to the present invention is however not alpha-galactosylceramide.

According to the present invention, said heterologous lipid carrier is intended to be stable against conditions encountered in the gut (e.g. bile salt), since the association of said carrier with an exterior surface of said cell of said modified microorganism is stable under physiological conditions and thus resists a treatment with 0.3% bile salts, optionally in combination with pancreatine juice (pancreatin from porcine pancreas P3292-25G) in PBS for at least 1 hour at least 37° C., at least 37.5° C., at least 38° C., at least 38.5° C., such as at 37° C., 37.5° C., 38° C., 38.5° C. or more ° C. as it is further described in the Example section. Such stable association between the modified microorganism and the lipid carrier ensures that the product containing the modified microorganism is efficient, safe and exhibits less variability which is important for the product quality, in particular in the pharmaceutical field. A stable association is also important in order to fulfill the regulatory requirements.

In this context, the term “resistance” or “resist” means that at least 80%, at least 85%, at least 90%, at least 95%, such as 80%, 81%, 82%, 83%, 84%, 85%, 86%, 87%, 88%, 89%, 90%, 91%, 92%, 93%, 94%, 95%, 96%, 97%, 98%, 99%, or even 100% of the heterologous lipid carrier associated with an exterior surface of said cell of said modified microorganism as defined elsewhere herein remains associated after a treatment with 0.3% bile salts, optionally in combination with pancreatine juice in PBS or DPBS for at least 1 hour at least 37° C.

Further, exemplary non-limiting lipid carriers of the present invention may have one or more of the following characteristics: comprising a glyceride moiety; preferably said glyceride moiety comprises at least one fatty acid, further preferably said lipid portion of said lipid carrier comprises said glyceride moiety; comprising a ceramide moiety, wherein preferably: (a) said ceramide moiety is composed of an amino alcohol (e.g. sphingosine) and/or a fatty acid, further preferably said lipid portion of said lipid carrier comprises said ceramide moiety; and/or (b) said ceramide moiety is a β-anomerically linked moiety or α-anomerically linked moiety, most preferably said lipid portion of said lipid carrier comprises said ceramide moiety; comprising a carbohydrate moiety; preferably said non-lipid portion of said lipid carrier comprises said carbohydrate moiety; further preferably said carbohydrate moiety is ß- (e.g. the first sugars linked to ceramide are typically β-linked galactose (GalCer) or glucose (GIcCer)) or α-linked (e.g. for increasing immunity) to said ceramide moiety, further preferably said carbohydrate is not a monosaccharide or a disaccharide moiety; further preferably said carbohydrate is selected from the group consisting of: an oligosaccharide and a polysaccharide, most preferably the first sugar of the said carbohydrate is a galactose, a glucose, a mannose, a xylose, a neuraminic acid, a N-acetyl glucosamine, N-acetyl galactosamine or a galacturonic acid; comprising one or more polypeptides (e.g. a heterologous recombinant or fusion polypeptide, e.g. a glycosylated polypeptide or an immunologically active polypeptide); preferably said non-lipid portion comprising said one or more polypeptides; further preferably said one or more polypeptides is an enzyme, a cytokine or a chemokine, a peptidomimetic compound, an antigen, an antibody (e.g. a single chain or a single domain antibody), a fragment or derivative thereof; comprising a pharmaceutically active compound; preferably said non-lipid portion comprising said pharmaceutically active compound; is not comprising a recombinant and/or fusion polypeptide (e.g. an endogenous recombinant and/or fusion polypeptide), preferably said recombinant and/or fusion polypeptide is obtained by the means of artificial genetic manipulation; is not expressed or synthetized by said microorganism; is at least partially expressed or synthetized by said microorganism (e.g. endogenously synthetized), preferably said microorganism is capable of expressing or synthetizing a ceramide or sphingolipid moiety (e.g. a Sphingomonas sp. Gram-negative bacterium); said lipid portion is not comprising a polypeptide; is not comprising a transmembrane polypeptide or a polypeptide membrane anchor domain; is not susceptible to proteolysis (e.g. under physiological conditions); is not immunogenic to a mammalian host, preferably said mammalian host is human; is immunogenic to a mammalian host, preferably said mammalian host is human; is not covalently bound to said cell membrane of said cell of said modified microorganism; comprising a glycolipid; comprising a lipopeptide (e.g. a glycosylated lipopeptide).

In this context and as used herein, the term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. The terms “polypeptide” and “protein” also refer to naturally modified polypeptides/proteins wherein the modification is effected e.g. by post-translational modifications like glycosylation, acetylation, phosphorylation and the like. Such modifications are well known in the art.

Most preferably, the lipid carrier of the present invention is selected from the group consisting of:
i) Monosialotetrahexosylganglioside (GM1) or Monosialotetrahexosylganglioside red (GM1red; reductive Ozonized GM1) having the following formula

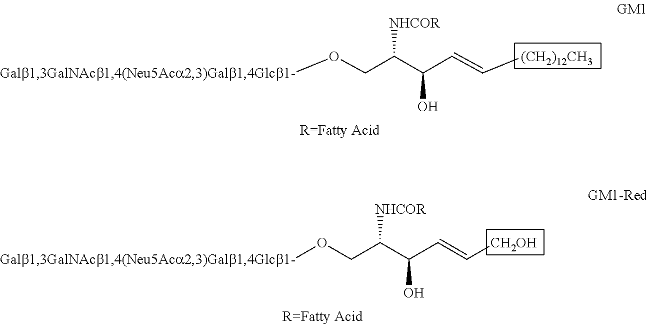

ii) Globotriaosylceramide (Gb3), a GM1-Gb3 chimera having the formula

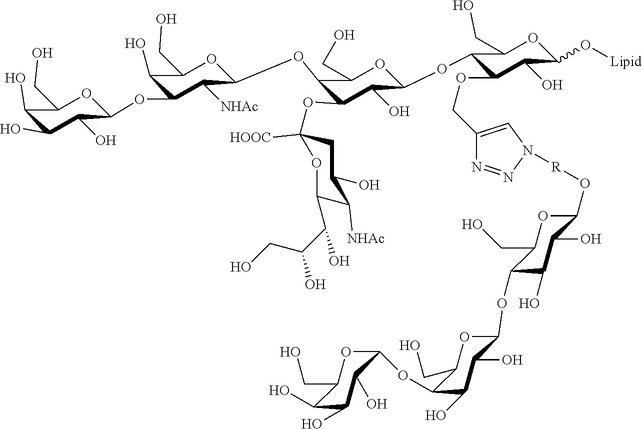

iii) Ganglioside GD1a,
iv) Gangliosides GM2, GD2, GD1b, GT1b, GT1c, GQ1c, GA1, GM1b,
v) Gangliosides GM3, GD3 and GT3,
vi) Gangliosides Gb4, Blood Group Type I, Type 2, Blood Group A, Blood Group B, Blood Group H, Blood Group H Type 1, Blood Group H Type 2, Blood Group H Type 3, Lewis y, Lewis a, Lewis b, Lewis x, H, Sialyl Lewis x, Sialyl Lewis a, Sialyl Lewis b, Sialyl Lewis x, alpha Gal epitope, Gal a1-3Galß1-4GlacNAc, Gal(α 1-4)Gal(ß1-4)GlcNAc-R, Gal(α 1-4)Gal(ß1-4)Glc NAc-(ß1-3)Gal(ß1-4)Glc-R, Gal(α 1-4)Gal(ß1-4)GlcNAc(ß1-2) Man-R
vii) any one of the following:

| Name | Structure |
|---|---|
| 2-6 Sialyl i-Lewis x | Neu5Ac(α 2-6)Gal(β1-4)GlcNAc(β1-3)Gal(β1-4)[Fuc(α 1-3)]GlcNAc(β1-)-R |
| 3′-Sulfo Lewis a | HSO3(-3)Gal(β1-3)[Fuc(a1-4)]GlcNAc(β1-)-R |
| 3′-Sulfo Lewis x | HSO3(-3)Gal(β1-4)[Fuc(a1-3)]GlcNAc(β1-)-R |
| 6,6′-Disulfo Sialyl Lewis x | Neu5Ac(α 2-3)[HSO3(-6)]Gal(β1-4)[Fuc(α 1-3)][HSO3(-6)]GlcNAc(β1-)-R |
| 6-Sulfo Lewis x | Gal(β1-4)[Fuc(α 1-3)][HSO3(-6)]GlcNAc(β1-)-R |
| 6′-Sulfo Sialyl Lewis x | Neu5Ac(α 2-3)[HSO3(-6)]Gal(β1-4)[Fuc(α 1-3)]GlcNAc(β1-)-R |
| 6^{(GlcNAc)}-su-SLe^{x} | Neu5Acα2-3Galβ1-4(Fucα1-3)(6-O-Su)GlcNAcβ-R |
| 6′-Sia-6-Su-LacNAc | Neu5Acα2-6Galβ1-4(6-O-Su)GlcNAcβ-R |
| 6-Su-3′SiaLe^{c} | Neu5Acα2-3Galβ1-3(6-O-Su)GlcNAcβ-R |
| 6-Su-3′SLN | Neu5Acα2-3Galβ1-4(6-O-Su)GlcNAcβ-R |
| 3′SLN(Gc) | Neu5Gcα2-3Galβ1-4GlcNAcβ-R |
| 6′SLN(Gc) | Neu5Gcα2-6Galβ1-4GlcNAcβ-R |
| GlcNAcβ3′LacNAc | GlcNAcβ1-3Galβ1-4GlcNAcβ-R |
| Isomaltotriose | Glcα1-6Glca1-6Glcβ-R |
| Chitotriose | GlcNAcβ1-4GlcNAβ1-4GlcNAcβ-R |
| alpha Gal epitope, | Gal(α 1-3)Gal(β1-4)GlcNAc-R |
| Arthro | GlcNAcβ1,3Manβ1,4Glcβ-R |
| Aₜᵣᵢ | GalNAcα1-3(Fucα1-2)Galβ-R |
| Bₜᵣᵢ | Galα1-3(Fucα1-2)Galβ-R |
| Gal₂3,4-GlcNAc | Galβ1-4(Galβ1-3)GlcNAcβ-R |
| asialo-GM1,GA1 | DGalp(β1-3)DGalpNAc(β1-4)DGalp(β1-4)DGlcp(β1-1)-R |
| asialo-GM2,GA2 | DGalpNAc(β1-4)DGalp(β1-4)DGlcp(β1-1)-R |
| Blood Group A | GalNAc(α 1-3)[Fuc(α 1-2)]Gal(β1-)-R |
| Trisaccharide | |
| Blood Group A | GalNAc(α 1-3)[Fuc(α 1-2)]Gal(β1-3)GlcNAc(β1-)-R |
| Type 1 | |
| Blood Group A | GalNAc(α 1-3)[Fuc(α 1-2)]Gal(β1-3)[Fuc(α 1-4)]GlcNAc(β1-)-R |
| Type 1 (difucosyl) | |
| Blood Group A | GalNAc(α 1-3)[Fuc(α 1-2)]Gal(β1-4)GlcNAc(β1-)-R |
| Type 2 | |
| Blood Group A | GalNAc(α 1-3)[Fuc(α 1-2)]Gal(β1-4)[Fuc(α 1-3)]GlcNAc(β1-)-R |
| Type 2 (difucosyl) | |
| Blood Group A | GalNAc(α 1-3)[Fuc(α 1-2)]Gal(β1-3)GalNAc(α 1-)-R |
| Type 3 | |
| Blood Group A | GalNAc(α 1-3)[Fuc(α 1-2)]Gal(β1-3)GalNAc(β1-)-R |
| Type 4 | |
| Blood Group B | Gal(α 1-3)[Fuc (α 1-2)]Gal(β1-3)GlcNAc(β1-3)Gal-R, |
| Blood Group B | Gal(α 1-3)[Fuc(α 1-2)]Gal(β1-4)GlcNAc(β1-)-R |
| Type 2 | |
| Blood Group H | Fuc(α 1-2)Gal(β1-3)GlcNAc(β1-)-R |
| Type 1, | |
| Blood Group H | Fuc(α 1-2)Gal(β1-4)GlcNAc(β1-)-R |
| Type 2, | |
| Blood Group H | Fuc(α 1-2)Gal(β1-3)GalNAc(α 1-)-R |
| Type 3, | |
| Blood Group H, | Fuc(α 1-2)Gal(β1-)-R |
| Sia6′H (type 2) | Neu5Acα2-6(Fucα1-2)Galβ1-4GlcNAcβ-R |
| 6-LacNAc-TF | Galβ1-4GlcNAcβ1-6(Galβ1-3)GalNAcα-R |
| C-Series Ganglio- | R-Gal(β1-3)GalNAc(β1-4)[Neu5Ac(α 2-8)Neu5Ac(α 2-8)Neu5Ac(α 2- |
| sides Oligo- | 3)]Gal(β1-4)Glc(β1-1)-R |
| saccharide/Ganglio- | |
| tetraosyl Core | |
| Structure | |
| C-Series Ganglio- | Neu5Ac(α 2-8)Neu5Ac(α 2-8)Neu5Ac(α 2-3)Gal-R |
| sides Oligo- | |
| saccharide/Hemato- | |
| or Ganglio-Type | |
| Cyclic Sialyl 6-Sulfo Lewis x | cyclicNeu5Ac(α 2-3)Gal(b1-4)[Fuc(α 1-3)][HSO3(-6)]GlcNAc-R |
| Dimeric Lewis x | Gal(β1-4)[Fuc(α 1-3)]GlcNAc(β1-3)Gal(b1-4)[Fuc(α 1-3)]GlcNAc(β1-)-R |
| Disialyl Lewis a | Neu5Ac(α 2-3)Gal(β1-3)[Neu5Ac(α 2-6)][Fuc(α 1-4)]GlcNAc(β1-)-R |
| Disialyl Lewis c | Neu5Ac(α 2-3)Gal(β1-3)[Neu5Ac(α 2-6)]GlcNAc(β1-)-R |
| F1 Alpha | Gal(b1-4)GlcNAc(b1-6)GalNAc(α 1-)Ser/Thr |
| fucosyl GM1 | (Fucal-2Galβ1-3GalNAcβ1-4[NeuAca2-3]-Galβ1-4Glcβ1-I-R |
| GA1: (Gg4Cer) | Galβ1,3GalNAcβ1,4Galβ1,4Glcβ1-R |
| GA2: (Gg3Cer) | GalNAcβ1,4Galβ1,4Glcβ1-R |
| Gal a1-3Galβ1-4GlacNAc | Gal α 1-3Galβ1-4GlacNAc-R |
| Galili,Gala-3′LacNAc | Galα1-3Galβ1-4GlcNAcβ-R |
| Gala | Gal α 1,4Galβ-R |
| GalCer: | Galβ1-R |
| GalNAc-GD1a: | GalNAcβ1,4Galβ1,3GalNAcβ1,4(Neu5Acα2,3)Galβ1,4Glcβ1-R |
| (IV3Neu5AcII3Neu5AcGg5Cer) | |
| Ganglio | Galβ1,3GalNAcβ1,4Galβ1,4Glcβ-R |
| GbOse3Cer: | Galα1,4Galβ1,4Glcβ1-R |
| (Gb3Cer) | |
| GbOse4Cer | GalNAcβ1,3Galα1,4Galβ1,4Glcβ1-R |
| GD1a: | Neu5Acα2,3Galβ1,3GalNAcβ1,4(Neu5Acα2,3)Galβ1,4Glcβ1-R |
| (IV3Neu5AcII3Neu5AcGg4Cer) | |
| GD1b: | Galβ1,3GalNAcβ1,4(Neu5Acα2,8Neu5Acα2,3)Galβ1,4Glcβ1-R |
| (II3(Neu5Ac)2Gg4Cer) | |
| GD1b-lactone: | II3[Neu5Ac-(2-8,1-9)-Neu5Ac]Gg4-R |
| GD1c: | Neu5Acα2,8Neu5Acα2,3Galβ1,3GalNAcβ1,4Galβ1,4Glcβ1-R |
| (IV3(Neu5Ac)2Gg4Cer) | |
| GD1α: | Neu5Ac α2,3Galβ1,3(Neu5Acα2,6)GalNAcβ1,4Galβ1,4Glcβ1-R |
| (IV3Neu5AcIII6Neu5AcGg4Cer) | |
| GD2: | GalNAcβ1,4(Neu5Acα2,8Neu5Acα2,3)Galβ1,4Glcβ1-R |
| (II3(Neu5Ac)2Gg3Cer) | |
| GD3: | Neu5Acα2,8Neu5Acα2,3Galβ1,4Glcβ1-R |
| (II3(Neu5Ac)2LacCer) | |
| GGal | Neu5Ac(α 2-3)DGalp(β1-1)-R |
| GlcCer: | Glcβ1-R |
| Globo | GalNAcβ1,3Gala1,4Galβ1,4Glcβ-R |
| Globo-H | Fuc α 2Galβ3GalNAcβ3Gal α 4Galβ4Glcβ1-R |
| Gb5 | Gal(β1-3)GalNAc(β 1-3)Gal(a 1-4)Gal(β 1-4)Glc-R |
| Gb5 | Galβ3GalNAcβ3Galα4Galβ4Glcβ1-R |
| monosialyl-Gb5 | SAα3Galβ3GalNAcβ3Galα4Galβ4Glcβ1-R |
| disialyl-Gb5 | SAα3Galβ3GalNAcβ3(SAa2-3)Galα4Galβ4Glcβ1-R |
| iso-Gb3 | Galα3Galβ4Glcβ1-R |
| iso-Gb4 | GalNAcβ3Galα3Galβ4Glcβ1-R |
| Forssman | GalNAcα3GalNAcβ3Galα4Galβ4Glcβ1-R |
| GM1a: r | Galβ1,3GalNAcβ1,4(Neu5Acα2,3)Galβ1,4Glcβ1-R |
| (II3Neu5AcGg4Cer) | |
| GM1b: | Neu5Acα2,3Galβ1,3GalNAcβ1,4Galβ1,4Glcβ1-R |
| (IV3Neu5AcGg4Cer) | |
| GM2: | GalNAcβ1,4(Neu5Acα2,3)Galβ1,4Glcβ1-R |
| (II3Neu5AcGg3Cer) | |
| GM2b | Neu5Ac(α 2-8)Neu5Ac(α 2-3)DGalp(β1-4)DGlcp(β1-1)-R |
| GM3: | Neu5Acα2,3Galβ1,4Glcβ1-R |
| (II3Neu5AcLacCer) | |
| GM4: | Neu5Acα2-3Galβ1-R |
| (I3Neu5AcαGalCer) | |
| GP1c: | Neu5Acα2,8Neu5Acα2,3Galβ1,3GalNAcβ1,4 |
| (IV3(Neu5Ac)2II3 | (Neu5Acα2,8Neu5Acα2,8Neu5Acα2,3)Galβ1,4Glcβ1-R |
| (Neu5Ac)3Gg4Cer) | |
| GP1cα: | Neu5Ac |
| (IV3Neu5AcIII6Neu5Ac,II3(Neu5Ac)3Gg4Cer) | α2,3Galβ1,3(Neu5Acα2,6)GalNAcβ1,4(Neu5Acα2,8Neu5Acα2,8Neu5Acα2,3)Galβ1,4Glcβ1-R |
| GQ1b: | Neu5Acα2,8Neu5Acα2,3Galβ1,3GalNAcβ1,4(Neu5Acα2,8Neu5Acα2,3)Galβ1,4Glcβ1-R |
| (IV3(Neu5Ac)2II3 | |
| (Neu5Ac)2Gg4Cer) | |
| GQ1bα: | Neu5Ac |
| (IV3(Neu5Ac)2III6(Neu5Ac)2Gg4Cer) | α2,3Galβ1,3(Neu5Acα2,6)GalNAcβ1,4(Neu5Acα2,8Neu5Acα2,3)Galβ1,4Glcβ1-R |
| GQ1c: | Neu5Acα2,3Galβ1,3GalNAcβ1,4(Neu5Acα2,8Neu5Acα2,8Neu5Acα2,3)Galβ1,4Glcβ1-R |
| (IV3Neu5AcII3(Neu5Ac)3Gg4Cer) | |
| GT1,GT1b | Neu5Ac(α 2-3)DGalp(β1-3)DGalNAc(β1-4)[Neu5Ac(α 2-8)Neu5Ac(α |
| | 2-3)]DGalp(β1-4)DGlcp(β1-1)-R |
| GT1a: | Neu5Acα2,8Neu5Acα2,3Galβ1,3GalNAcβ1,4(Neu5Acα2,3)Galβ1,4Glcβ1-R |
| N(V3(Neu5Ac)2II3Neu5AcGg4Cer) | |
| GT1b: | Neu5Acα2,3Galβ1,3GalNAcβ1,4(Neu5Acα2,8Neu5Acα2,3) Galβ1,4Glcβ1-R |
| (IV3Neu5AcII3(Neu5Ac)2Gg4Cer) | |
| GT1c: | Galβ1,3GalNAcβ1,4(Neu5Acα2,8Neu5Acα2,8Neu5Acα2,3)Galβ1,4Glcβ1-R |
| (II3(Neu5Ac)3Gg4Cer) | |
| GT1a: | Neu5Ac α2,3Galβ1,3(Neu5Acα2,6)GalNAcβ1,4(Neu5Acα2,3)Galβ1,4Glcβ1-R |
| (IV3Neu5AcIII6(Neu5Ac)2Gg4Cer) | |
| GT2: | GalNAcβ1,4(Neu5Acα2,8Neu5Acα2,8Neu5Acα2,3)Galβ1,4Glcβ1-R |
| GT3: | Neu5Acα2,8Neu5Acα2,8Neu5Acα2,3Galβ1,4Glcβ1-R |
| (II3(NeuAc)3LacCer) | |
| Internal Lewis x | Gal(b1-4)GlcNAc(b1-3)Gal(b1-4)[Fuc(a1-3)]GlcNAc(b1-3)Gal(b1-4)GlcNAc(b1- |
| | 3)Gal(b1-4)GlcNAc(b1-3)Gal(b1-4)Glc(b1-1)-R |
| Isoglobo | GalNAcβ1,3Gala1,3Galβ1,4Glcβ-R |
| LacCer: | Galβ1,4Glcβ1-R |
| Lacto | Galβ1,3GlcNAcβ1,3Galβ1,4Glcβ-R |
| Lewis a, | Gal(β1-3)[Fuc(α 1-4)]GlcNAc(β1-)-R |
| Lewis b, | Fuc(α 1-2)Gal(β1-3)[Fuc(α 1-4)]GlcNAc(β1-)-R |
| Lewis x,H, | Gal(β1-4)[Fuc(α 1-3)]GlcNAc(β1-)-R |
| Lewis y, | Fuc(α 1-2)Gal(β1-4)[Fuc(α 1-3)]GlcNAc(β1-)-R |
| Mollu | Fuc α 1,4GlcNAcβ1,2Man α 1,3Manβ 1,4Glcβ-R |
| Muco | Galβ1,3Galβ1,4Galβ1,4Glcβ-R |
| N-Acetyl GD3 | Neu5Ac(α 2-8)Neu5Ac(α 2-3)Gal(b1-4)Glc(b1-1)-R |
| Neogala | Galβ 1,6Galβ 1,6Galβ-R |
| Neolacto | Galβ1,4GlcNAcβ1,3Galβ1,4Glcβ-R |
| Neu5Ac(a2- | Neu5Ac(α 2-3)Gal(β1-)-R |
| 3)Gal(b1) | |
| Neu5Ac(a2- | Neu5Ac(α 2-3)Gal(β1-3)GalNAc-R |
| 3)Gal(b1- | |
| 3)GalNAc | |
| Neu5Ac(a2- | Neu5Ac(α 2-8)Neu5Ac(α 2-3)Gal-R |
| 8)Neu5Ac(a2- | |
| 3)Gal | |
| (Sia)₃ | Neu5Acα2-8Neu5Acα2-8Neu5Acα-R |
| 3′-SL | Neu5Acα2-3Galβ1-4Glcβ-R |
| N-Glycolyl GM3 | Neu5Gc(α 2-3)Gal(β1-4)Glc(β1-1)-R |
| NOR1 | Gal(α 1-4)GalNAc(β 1-3)Gal(α a 1-4)Gal(β 1-4)Glc-R |
| NOR2 | Gal(α 1-4)GalNAc-(β 1-3)Gal(α 1-4)GalNAc(β 1-3)Gal(α 1-4)Gal(β 1-4)Glc-R |
| NOT int | GalNAc(β 1-3)Gal(α 1-4)GalNAc(β1-3)Gal-(α 1-4)Gal(β 1-4)Glc-R |
| O antigen | Fuc (α 1-2)Gal(β1-3)GlcNAc(β1-3)Gal-R, |
| OAc-GT1b | Neu5Ac(α 2-3)DGalp(β1-3)DGalNAc(β1-4)XNeu5Ac9Ac(α 2-8)Neu5Ac(α 2- |
| | 3)]DGalp(β-4)DGlcp(β1-1)-R |
| P antigen (Gb4) | Gal(α 1-4)Gal-(β 1-4)GlcNAc(β 1-3)Gal(β 1-4)Glc-R |
| Pk Antigen (Gb3) | Gal(α 1-4)Gal(β1-4)Glc-R |
| Pi | Galα1-4Galβ1-4GlcNAcβ-R |
| Schisto | GalNAcβ1,4Glcβ-R |
| Sialyl Lewis a, | Neu5Ac(α 2-3)Gal(β1-3)[Fuc(α 1-4)]GlcNAc(β1-)-R |
| Sialyl Lewis c | Neu5Ac(α 2-3)Gal(β1-3)GlcNAc(β1-)-R |
| Sialyl Lewis x, | Neu5Ac(α 2-3)Gal(β1-4)[Fuc(α 1-3)]GlcNAc(β1-3)Gal(β1-4)GlcNAc(β1-3)Gal(β1-)-R |
| Sialyl Lewis x, | Neu5Ac(α 2-3)Gal(β1-4)[Fuc(α 1-3)]GlcNAc(β1-)-R |
| Sialyl Lewis x-i | Neu5Ac(α 2-3)Gal(β1-4)[Fuc(α 1-3)]GlcNAc(β1-3)Gal(β1-4)GlcNAc(β1-3)Gal(β1-)-R |
| LNT | Galβ1-3GlcNAcβ1-3Galβ1-4Glcβ-R |
| LNnT | Galβ1-4GlcNAcβ1-3Galβ1-4Glcβ-R |
| Sialyl-TF | Neu5Ac(α 2-6) Gal (β1-3) α GalNAc-R, |
| sialyl-Tn | Neu5Ac(α 2-6)GalNAc-R, |
| sLac | NeuAc-Gal0-3GicNAcB-3GalB-4Glc-R |
| Spirometo | Galβ1,4Glcβ1,3Galβ-R |
| Sulfatide: | Sulfate3Galβ1-R |
| TF/Core-1 | α Gal (β1-3)aGalNAc-R, |
| 3′-sialyl-TF | Neu5Acα2-3Galβ1-3GalNAcα-R |
| Tn | α GalNAc-R |
| 6-SiaβTF | Neu5Acβ2-6(Galβ1-3)GalNAcα-R |
| 3-LacNAc-Tₙ | Galβ1-4GlcNAβ1-3GalNAcα-R |
| 6-LacNAc-Tₙ | Galβ1-4GlcNAβ1-6GalNAcα-R |
| 6′SLN | Neu5Acα2-6Galβ1-4GlcNAcβ-R |
| Core 2 | GlcNAcβ1-6(Galβ1-3)GalNAcα-R |
| Core 4 | GlcNAcβ1-3(GlcNAcβ1-6)GalNAcα-R |
| Trifucosyl-Lewis b Antigen | Fuc(α 1-2)Gal(b1-3)[Fuc(α 1-4)]GlcNAc(b1-3)Gal(b1-3)[Fuc(α 1-4)]GlcNAc(b1-)-R |
| Trifucosyl-Lewis y Antigen | Fuc(α 1-2)Gal(b1-4)[Fuc(α 1-3)]GlcNAc(b1-3)Gal(b1-4)[Fuc(α 1-3)]GlcNAc(b1-)-R |
| Type 1 | (GalNAc(α 1-3)[Fuc(α 1-2)]Gal(β1-3)GlcNAc[Fuc(α 1-4)]-R), |
| Type 1 A | GalNAc(α1-3)[Fuc(α 1-2)]Gal(β1-3)GlcNAc(β1-3)Gal-R, |
| Type 2 A | GalNAc(α1-3)[Fuc(α 1-2)]Gal(β1-4)GlcNAc-R, |
| Type 3 A | GalNAc(α1-3)[Fuc(α 1-2)]Gal(β1-3)GalNAc(β1-3)Gal[Fucα 1-2)-R, |
| Type 4 A | GalNAc(α1-3)[Fuc(α 1-2)]Gal(β1-3)GalNAc(β1-3)Gal(a 1-4)Gal(β 1-4)Glc--R, |
| VIM-2 | Neu5Ac(α 2-3)Gal(β1-4)GlcNAc(β1-3)Gal(β1-4)[Fuc(α-3)]GlcNAc(β1-)-R |
| Type 4 A | GalNAc(α1-3)[Fuc(α 1-2)]Gal(β1-3)GalNAc(β1-3)Gal(a 1-4)Gal(β 1-4)Glc--R, |
| Man₃ | Manα1-3(Manα1-6)Manα-? |
| 3′SLN | Neu5Acα2-3Galβ1-4GlcNAcβ-R |
| 6′SL | Neu5Acα2-6Galβ1-4Glcβ-R |
| Human milk oligosaccharide | see WO 2012/092153, WO 2010/120682, WO 2005/055944, U.S. Pat. No. 5,945,314 |

wherein R is one or more of the following: a carbohydrate/s, a peptide/s, a lipid/s, a linker/s and a chemical compound/s or substance/s or molecule/s; or R comprises one or more of the following: a carbohydrate/s, a peptide/s, a lipid/s, a linker/s or a chemical compound/s or substance/s or molecule/s; further, any one of lipid carriers as defined herein further coupled to TF disaccharide, Core-1 structure, Tn monosaccharide, Sialyl-TF mono- or disialylated, Sialyl-Tn, Polysialic acid, or mannose-6-phosphate moiety; any one of lipid carriers as defined herein further coupled to N-Glycan or O-glycan moiety (e.g. as can be found on glycoproteins or glycopeptides); any one of lipid carriers as defined herein further coupled to a carbohydrate moiety; a truncated or elongated derivative of any one of lipid carriers as defined herein; a phosphorylated, sulfated or acetylated derivative of any one of lipid carriers as defined herein; derivatives and analogs of any of any one of lipid carriers as defined herein. According to the present invention said heterologous lipid carrier of said modified microorganism is however not alpha-galactosylceramide.

A linker according to the present invention may refer to one or more linkers and/or one or more branched linker(s). Such branched linker(s) allow(s) coupling directly or via another linker(s) of one or more moieties such as carbohydrate(s), peptide(s) or protein(s) and of a core structure as defined elsewhere herein such as GM1, a derivative thereof or a carbohydrate structure of GM1. Said linker according to the present invention may comprise one or more carbohydrate(s), or peptide(s). Said linker may be any one of a biotin, NAc-CH2-NAc-CH2-(1.4-Tz)-(CH2)₂-EG3-NH—, an aminopropyl group, N-acetly-propargyl, or a thiazole group.

As used herein, the term “glyceride” may refer to esters of glycerol (propane-1,2,3-triol) with fatty acids, widely distributed in nature. They are by long-established custom subdivided into triglycerides, 1,2- or 1,3-diglycerides, and 1- or 2-monoglycerides, according to the number and position of acyl groups (not, as one might suppose, the number of glycerol residues). The recommended method for naming individual glycerides is mono-, di- or tri-O-acylglycerol, as appropriate.

As used herein, the term “ceramide-like glycolipid” (used interchangeably with “ceramide-like glycolipid moiety”) may refer to a ceramide derivative comprising a ceramide moiety and a saccharide moiety or a β- or α-linked sacharide derivative of ceramide or analog thereof (cf. WO2010081026), wherein said β- or α-linked sacharide derivative of ceramide or analog thereof may comprise Formula I:

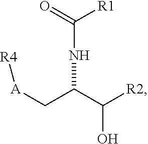

wherein R1 is a linear or branched C1-C80 (e.g., C1-C36, C27, C2-C36, C3-C36, C4-C36, or C16-C24) alkane or C2-C80 (e.g., C2-C36, C2-C27, C3-C36, C4-C36 or C16-C24) alkene; or R1 is —C(OH)—R3, wherein R3 is a linear or branched C1-C80 (e.g., C1-C26, C1-C36, C2-C36, or C16-C24) alkane or C2-C80 (e.g., C2-C36, C2-C26 or C16-C24) alkene; or R1 is a C6-C80 (e.g., C6-C36, C6-C27 or C16-C24) alkane or alkene, wherein:
(i) C6-C80 (e.g., C6-C36, C6-C27 or C16-C24) alkane or alkene is substituted with a C5-C15 cycloalkane, C5-C15 cycloalkene, heterocycle, or aromatic ring; or
(ii) C6-C80 (e.g., C6-C36, C6-C27 or C16-C24) alkane or alkene includes, within the C6-C80 alkyl or alkenyl chain, a C5-C15 cycloalkane, C5-C15 cycloalkene, heterocycle, or aromatic ring;
R2 is one of the following: —CH₂(CH₂)ₓCH₃, —CH(OH)(CH₂)ₓCH₃, —CH(OH) CH(OH)(CH₂)ₓCH₃, CH═CHCH₂OH, —CH(OH)(CH₂)ₓCH(CH₃)₂, —CH═CH(CH₂)ₓCH₃, —CH(OH)(CH₂)ₓCH(CH₃)CH₂CH₃, wherein X is an integer ranging from 4-17; R2 can also be adamantaneacetyl or a sphingosine mimicry. R4 is an α-linked or a β-linked disacharide or polysacharide moiety (e.g., comprising 3 or more saccharide moieties (e.g., 3 to 5 sacharide moieties (e.g., as in GM1), e.g, comprising one or more repeat/s of the same disacharide or polysacharide moiety, or comprising one or more linker/s, peptide/s, chemical compound/s, preferably comprising 3 to 100 sacharide moieties, further preferably comprising 3 to 30 sacharide moieties, most preferably comprising 3 to 12 sacharide moieties), or when R1 is a linear or branched C1-C80 (e.g., C1-C36, C1-C27, C2-C36, C3-C36, C4-C36, or C16-C24) alkane, R4 is:

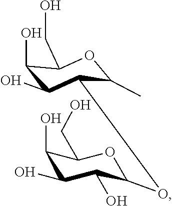

and A is O or —CH₂; In a preferred embodiment, the R4 (e.g., an epitope, e.g., an epitope for a toxin) may comprise 3 to 5 sacharide moieties (i.e. sugars, e.g., as in GM1), wherein the binding capacity of the lipid carrier of the present invention may be optionally increased in that the polysaccharide moiety may comprise several repeats of the same R4 (e.g., epitope), wherein said R4 may comprise more than 12 saccahride moities.

As used herein, the term “ceramide” (or “ceramide moiety”) may refer to compound or substance composed of sphingosine and a fatty acid.

As used herein, the term “sphingolipid” may refer to a class of lipids containing a backbone of sphingoid bases and a set of aliphatic amino alcohols that includes sphingosine or a substance structurally similar to it. Non-limiting examples of sphingolipids include: sphingosine, sphingomyelins, cerebroside, sulfatides, globosides, and gangliosides.

As used herein, the term “sphingosine” (or “sphingosine moiety”) may refer to 2-amino-4-trans-octadecene-1,3-diol), which is an 18-carbon amino alcohol with an unsaturated hydrocarbon chain, which forms a primary part of sphingolipids, a class of cell membrane lipids that include sphingomyelin, an important phospholipid.

As used herein, the term “ganglioside” may refer to a molecule composed of a glycosphingolipid (i.e. a subtype of glycolipids containing the amino alcohol sphingosine, e.g. ceramide and oligosaccharide) with one or more sialic acids (e.g. n-acetylneuraminic acid, NANA) linked on the sugar chain.

As used herein, the term “fatty acid” (used interchangeably with “fatty acid moiety”) may refer to aliphatic monocarboxylic acids derived from or contained in esterified form in an animal or vegetable fat, oil or wax. Natural fatty acids commonly have a chain of 4 to 28 carbons (usually unbranched and even-numbered), which may be saturated or unsaturated. By extension, the term also embraces all acyclic aliphatic carboxylic acids and very long chains over 28 carbons (up to 80).

As used herein, the term “phospholipids” may refer to lipids containing phosphoric acid as mono- or di-esters, including phosphatidic acids and phosphoglycerides.

As used herein, the term “steroid” (used interchangeably with “steroid moiety”) may refer to naturally occurring compounds and synthetic analogs, based on the cyclopenta[a]phenanthrene carbon skeleton, partially or completely hydrogenated; there are usually methyl groups at C-10 and C-13, and often an alkyl group at C-17. By extension, one or more bond scissions, ring expansions and/or ring contractions of the skeleton may have occurred.

As used herein, the term “lipopeptide” may refer to a molecule comprising a peptide part (e.g. a peptide molecule as described herein) associated with (e.g. coupled to) a lipid part (e.g. a lipid molecule as described herein). Preferably said peptide part of the lipopeptide is linked directly to said lipid part of the lipopeptide via a covalent bond or a linker or linking molecule (e.g. a peptide-based linker or a chemical linker such as e.g. bi-functional linkers (NHS-ester and maleimid), copper-free click-chemistry alkyne-azido triazole linkages, unnatural amino acids, carbohydrate-mediate linkages, photocross-linkers. Other non-covalent linkages allowing an association (e.g. a stable association) between said peptide part and said lipid part are also within the scope of the present invention. The peptide of the peptide part can be of any length and may include one or more modifications, preferably post-translational modifications, including, but not limited to, glycosylation, sulfation, carboxylation, phosphorylation etc. The peptide part can also be a protein composed of one or more covalently or non-covalently associated polypeptide chains. The peptide can be covalently or non-covalently coupled to an additional chemical molecule. Each polypeptide chain may independently be modified, preferably post-translationally or chemically modified. In a preferred embodiment the peptide part is a binding molecule. Further preferably, the peptide part comprises an antibody, a fragment thereof or mutated or modified version. In another preferred embodiment the peptide can be a molecule which mimics a carbohydrate. Those molecules may comprise one or more peptide-based linkers. In another preferred embodiment the peptide part comprises a lectin or a fragment thereof. In another preferred embodiment the peptide part comprises an immunologically active molecule such as a cytokine or chemokine or a fragment thereof. In a preferred embodiment the peptide part binds to a toxin of the invention, a toxin receptor, a receptor, a cell, a protein, an immunologically active molecule, an inflammatory molecule or another molecule. The peptide part of the lipopeptide can also be any other naturally occurring, therefrom derived, or chemically synthetized chemical moiety able to bind to a toxin of the invention, a toxin receptor, a receptor, a cell, a protein, a carbohydrate or another molecule, or an immunologically active molecule. In a certain embodiment such molecules comprise DNA or RNA or a DNA or RNA peptide or protein complex. In further embodiments peptide/protein based binding molecules, which are modified to keep/stabilize their binding structure in environments like the gastrointestinal tract, Lung, urogenital tract better than the non-modified form, are used. Modifications, e.g. mutation of sites prone for proteolysis or of sites which when mutated help to stabilize the spacial three-dimensional structure, may also be used. Technologies to achieve this are known in the art. An advantage will be the generation of phage-display libraries displaying such molecules for selection of binders to toxins and/or pathogens, which is within scope of the present invention. Various lipid molecules of different type, length of fatty acid chains, with or without natural and chemical modifications may be used in the present invention. The sequence of the peptide/protein part may be designed accordingly and tested with suitable amino acids and side chains for covalent coupling. Chemical, peptide and carbohydrate-based spacers may be used for a better presentation on the bacteria. The coupling of the peptides with lipids can be for example carried on by the use of bi-functional linkers (e.g. NHS-ester and maleimid), copper-free click-chemistry alkyne-azido triazole linkages, unnatural amino acids, carbohydrate-mediate linkages, photocross-linkers a.o., which readily known in the art.

As used herein, the term “peptidomimetic compound” includes a peptide, which mimics a biological effect or chemical structure of another peptide, carbohydrate or a chemical entity; as well as a chemical entity, which mimics a peptide- or protein structure, epitope or determinant.

As used herein, the term “carbohydrate” (used interchangeably with “carbohydrate moiety”) may refer to compounds such as aldoses and ketoses having the stoichiometric formula Cn(H2O)n (e.g. hence “hydrates of carbon”). The generic term “carbohydrate” includes, but is not limited to, monosaccharides, oligosaccharides and polysaccharides as well as substances derived from monosaccharides by reduction of the carbonyl group (alditols), by oxidation of one or more terminal groups to carboxylic acids, or by replacement of one or more hydroxy group(s) by a hydrogen atom, an amino group, thiol group or similar groups. It also includes derivatives of these compounds. Preferably, said carbohydrate moiety of said non-lipid portion is a sialic acid residue.

As used herein, the term “receptor” or “toxin receptor” may refer to a toxin-binding molecule present on the surface of a cell, preferably of a mammalian cell. It may refer to a molecule present on the surface of a cell, preferably of a mammalian cell, which enables or facilitates the binding of a pathogenic microorganism or pathogen to said cell. Cells of interest include, for example epithelial or endothelial cells, in particular those that are part of a mammalian mucosal membrane or epithelia, such as human or animal mucosal membranes. Toxins for which receptors are described include but are not limited to Shiga toxin Stx1, Stx2, Stx2c, Stx2d, Stx2e, Stx2f, C. difficile toxin A, C. difficile toxin B, C. botulinum toxin, Vibrio cholerae toxin, E. coli heat labile enterotoxin Type 1, Escherichia coli heat-stable enterotoxin, Clostridium perfringens enterotoxin. Such toxin receptors include, but are not limited to, GUCY2C (guanylate cyclase 2C (heat stable enterotoxin receptor)), Heat labile enterotoxin- and Cholera Toxin-GM1 Ganglioside Receptor, Clostridium perfringens enterotoxin-receptors Claudin-3 and Claudin-4, Clostridium difficile A and B-receptors Combined Repetitive OligoPeptides (CROP'S), Clostridium difficile A receptor ß-type trisaccharide αGal(1,3)-ß Gal(1,4)-ß GlcNAc, Shiga toxin Stx1, Stx2, Stx2c, Stx2d Glycolipid receptor Globotriaosylceramide (Gb3) or Shiga toxin Stx2e Glycolipid receptor Globotetraosyl ceramide (Gb4).

As used herein, the term “toxin” includes toxins in their naturally occurring form, inactivated toxins and fragments or derivatives of toxins such as recombinant toxins of a pathogenic microorganism, for example pathogenic microorganisms. Toxins in connection with present invention are preferably toxins which are relevant for endangering health and/or well-being of humans or non-human animals, such cattle, pig, horse, sheep, goat, cats, dogs, ducks, goose, chicken, fish, etc. A toxin is preferably a toxin produced either by a bacterium belonging to a family selected from the group consisting of Enterobacteriaceae, Clostridiaceae, Vibrionaceae, Staphylococcaceae, Streptococcaceae, Helicobacteraceae, Pseudomonadaceae, Pasteurellaceae, Chlamydiaceae, Campylobacteraceae, Aeromonadaceae, Neisseriaceae, Listeriaceae, Corynebacteriaceae, Aeromonadales, Bacteroidaceae, Bordetella, Bacillaceae or a protozoa belonging to a family selected from the group consisting of Acanthamoebidae, Amoebida, Hexamitidae, Cryptosporidiidae or a fungi belonging to a family selected from the group consisting of Saccharomycetaceae, Trichocomaceae, Clavicipitaceae, Nectriaceae. Furthermore “toxin” may refer herein preferably to a toxin produced either by a bacterium belonging to a genus selected from the group consisting of Enterobacter, Echerischia, Shigella, Clostridium, Vibrio, Staphylococcus, Streptococcus, Helicobacter, Pseudomonas, Haemophilus, Chlamydia, Campylobacter, Salmonella, Citrobacter, Yersinia, Pasteurella, Neisseria, Listeria, Corynebacterium, Klebsiella, Aeromonas, Serratia, Proteus, Bacteroides, Bordetella, Bacillus or a protozoa belonging to a genus selected from the group consisting of Acanthamoeba, Entamoeba, Giardia, Cryptosporidium or a fungi belonging to a genus selected from the group consisting of Candida, Penicillium, Aspergillus, Claviceps, Paecilomyces, Fusarium. Furthermore, toxin includes toxins made in the gut. Preferably “toxin” may refer to an enterotoxin produced by a pathogenic microorganism. Within the context of the present invention the term “toxin” includes, but is not limited to toxins of the following list: E. coli: Heat labile toxin (LT), Heat stabile toxin (ST), Verotoxinsl Shiga like toxins (Stxs), Cytotoxins, endotoxins (LPS), EnteroAggregative ST toxin (EAST), Shigella: Shiga toxin (STxs), Shigella enterotoxins 1 (ShETI), Shigella enterotoxins 2 (ShET2), Neurotoxin; Salmonella: Cytolethal distending toxins (Cdt), AvrA toxin; Yersinia: Cytotoxic necrotizing facto (CNFy), Yersinia murine toxin (Ymt), Yst toxin, Toxin complex (TCa), Heat stabile toxin; Enterobacter. E. cloacae leukotoxin, Shiga-like toxin II, Klebsiella: heat-stable like enterotoxins, extracellular toxic complex (ETC); Serratia: Hemolysins (Shp, Pore-forming Toxin (PFT), Proteus: a-hemolysin (HlyA), Citrobacter heat-stable like toxin, Cytotoxins; Clostridium: C. perfringens alpha-toxin (CpPLC), C. perfringens beta toxin, C. perfringens enterotoxin (CPE), C. difficile enterotoxins (Tcd), C. butulinum Neurotoxins, C. tetani Tetanospasmin, C. butulinum C2 toxin, C. butulinum C3 toxin, C. perfringens epsilon-toxin (etoxin), C. perfringens iota-toxin (L-toxin), tetanus neurotoxin (TeNT), theta-toxinlPFO (perfringolysin 0), C. spiroforme (spiroforme toxin), C. septicum (a-toxin), Lecithinase; Vibrio: Cholera toxins (CTx), accessory cholera enterotoxin (Ace), RTX toxin, zona occludens toxin (Zot), Cholix toxin; Staphylococcus: a-hemolysin, P-hemolysin, 6-hemolysin, y-hemolysin, Exfoliative toxins (Exofoliatins), Panton-Valentine leukocidin (PVL), staphylococcal enterotoxins (SE), Toxic shock syndrome toxin-I (TSST-1) Streptococcus: P-haemolysinlcytolysin, CAMP factor, Streptolysin 0, Streptolysin S, Pneumolysin, S. pyogenes Exotoxins (PSE), Helicobacter vacuolating cytotoxin A (VacA), Cytolytic toxins Pseudomonas: Exotoxins (ex: ExoA, ExoS, ExoT, ExoU, ExoY), Phospholipase C (PLC) Pasteurella: Pasteurella Multocida Toxin (PMT), RTX toxins Bacillus: B. weihenstephanensis endotoxins, B. cereus Hemolysin BL (Hbl), B. cereus, onhemolytic Enterotoxin (Nhe), B. cereus Cytotoxin K (CytK), B. cereus emetic toxin, B. cereus toxin (Cereolysin), B. anthracis (Anthrax toxin), B. thuringiensis 6-endotoxins (Cry toxins), Campylobacter. Cytolethal distending toxin (cdtA, cdtB, cdtC), cholera-like enterotoxin Aeromonas: Aerolysin Cytotoxic Enterotoxin (ACT), ADP-ribosylation toxin, a-hemolysins, bhemolysins, Heat labile toxin (LT+), Heat stabile toxin (ST+) Neisseria: endotoxins (LPS) Bordetella: B. pertussis (pertusis toxin), Adenylate cyclase toxin, Tracheal cytotoxin, Dermonecrotic (heat-labile) toxin, endotoxins (LPS) Haemophilus: Endotoxin (LOS), Cytolethal distending toxins (HdCDT), Hemolysins Chlamydia: Endotoxins Corynebacteria: Cytotoxins, Diphteria toxin, Exotoxins Bacteroides: Bacteroides fragilis toxin (bft) Listeria: Listeriolysin 0.

In a preferred embodiment, the toxin is Heat labile toxin (LT), Heat stabile toxin (ST), Verotoxinsl Shiga like toxins (Stxs), Cytotoxins, endotoxins (LPS), EnteroAggregative ST toxin (EAST), Shiga toxin (STxs), Shigella enterotoxins 1 (ShETI), Shigella enterotoxins 2 (ShET2), Neurotoxin, Cytolethal distending toxins (Cdt), AvrA toxin, Cytotoxic necrotizing factor (CNFy), Yersinia murine toxin (Ymt), Yst toxin, Toxin complex (TCa), Heat stabile toxin, E. cloacae leukotoxin, Shiga-like toxin II, heat-stable like enterotoxins, extracellular toxic complex (ETC), Hemolysins (Shp, Pore-forming Toxin (PFT), a-hemolysin (HlyA), heat-stable like toxin, Cytotoxins, C. perfringens alpha-toxin (CpPLC), C. perfringens beta toxin, C. perfringens enterotoxin (CPE), C. difficile enterotoxins (Tcd), C. butulinum Neurotoxins, C. tetani Tetanospasmin, C. butulinum C2 toxin, C. butulinum C3 toxin, C. perfringens epsilontoxin (etoxin), C. perfringens iota-toxin (L-toxin), tetanus neurotoxin (TeNT), theta-toxinIPF0 (perfringolysin 0), C. spiroforme (spiroforme toxin), C. septicum (a-toxin), Lecithinase, Cholera toxins (CTx), accessory cholera enterotoxin (Ace), RTX toxin, zona occludens toxin (Zot), Cholix toxin, a-hemolysin, P-hemolysin, 6-hemolysin, y-hemolysin, Exfoliative toxins (Exofoliatins), Panton-Valentine leukocidin (PVL), staphylococcal enterotoxins (SE), Toxic shock syndrome toxin-I (TSST-I), P-haemolysinlcytolysin, CAMP factor, Streptolysin 0, Streptolysin S, Pneumolysin, S. pyogenes Exotoxins (PSE), vacuolating cytotoxin A (VacA), Cytolytic toxins, Exotoxins (ex: ExoA, ExoS, ExoT, ExoU, ExoY), Phospholipase C (PLC), Pasteurella Multocida Toxin (PMT), RTX toxins, B. weihenstephanensis endotoxins, B. cereus Hemolysin BL (Hbl), B. cereus, onhemolytic Enterotoxin (Nhe), B. cereus Cytotoxin K (CytK), B. cereus emetic toxin, B. cereus toxin (Cereolysin), B. anthracis (Anthrax toxin), B. thuringiensis 6-endotoxins (Cry toxins), Cytolethal distending toxin (cdtA, cdtB, cdtC), cholera-like enterotoxin, Aerolysin Cytotoxic Enterotoxin (ACT), ADP-ribosylation toxin, ahemolysins, b-hemolysins, Heat labile toxin (LT+), Heat stabile toxin (ST+), endotoxins (LPS), B. pertussis (pertusis toxin), Adenylate cyclase toxin, Tracheal cytotoxin, Dermonecrotic (heat-labile) toxin, endotoxins (LPS), Endotoxin (LOS), Cytolethal distending toxins (HdCDT), Hemolysins, Endotoxins, Cytotoxins, Diphteria toxin, Exotoxins, Bacteroides fragilis toxin (bft), Listeriolysin 0, or rota virus toxin (NSP4).

An “antibody” when used herein is a protein comprising one or more polypeptides (comprising one or more binding domains, preferably antigen binding domains) substantially or partially encoded by immunoglobulin genes or fragments of immunoglobulin genes. The term “immunoglobulin” (Ig) is used interchangeably with “antibody” herein. The recognized immunoglobulin genes include the kappa, lambda, alpha, gamma, delta, epsilon and mu constant region genes, as well as myriad immunoglobulin variable region genes.

In particular, an “antibody” when used herein, may typically be composed of two light (L) chains and two heavy (H) chains. Two types of light chain, termed lambda and kappa, may be found in antibodies. Depending on the amino acid sequence of the constant domain of heavy chains, immunoglobulins can be assigned to five major classes: A, D, E, G, and M. They may be further divided into subclasses (isotypes). The term “antibody” also includes, but is not limited to, but encompasses monoclonal, monospecific, poly- or multi-specific antibodies such as bispecific antibodies, humanized, chimeric, synthetic, recombinant, hybrid, mutated, grafted, and in vitro generated antibodies, with chimeric or humanized antibodies being preferred. The term “antibody” also includes scFvs, single chain antibodies, diabodies or tetrabodies, domain antibodies (dAbs) and nanobodies and parts thereof. In terms of the present invention, the term “antibody” shall also comprise multifunctional antibodies having several antigen binding sites.

Furthermore, the term “antibody” as employed in the invention also relates to derivatives of the antibodies (including fragments) described herein. A “derivative” of an antibody comprises an amino acid sequence which has been altered by the introduction of amino acid residue substitutions, deletions or additions. Additionally, a derivative encompasses antibodies which have been modified by a covalent attachment of a molecule of any type to the antibody or protein. Examples of such molecules include sugars, PEG, hydroxyl-, ethoxy-, carboxy- or amine-groups but are not limited to these. In effect the covalent modifications of the antibodies lead to the glycosylation, pegylation, acetylation, phosphorylation, amidation, without being limited to these. In a preferred embodiment the amino acid sequence is mutated to stabilize the protein part of the lipopeptide especially at its site of action in the mammalian, such as the gastrointestinal system or parts of it.

As used herein, the terms “effective amount” or “therapeutically effective amount,” refer to an amount of an active agent (e.g. modified microorganism) as described herein that is sufficient to achieve, or contribute towards achieving, one or more desirable clinical outcomes. An appropriate “effective” amount in any individual case may be determined using standard techniques known in the art, such as a dose escalation study.

As used herein, “administering” a compound (or microorganism) can be affected or performed using any of the various methods and delivery systems known to those skilled in the art. The administering can be performed, for example, intravenously, orally, nasally, via the cerebrospinal fluid, via implant, transmucosally, transdermally, intramuscularly, intraocularly, parenterally, topically, via inhalation and subcutaneously. The following delivery systems, which employ a number of routinely used pharmaceutically acceptable carriers, are only representative of the many embodiments envisioned for administering compositions according to the instant methods.

The appropriate dosage, or therapeutically effective amount, will depend on the condition to be treated, the severity of the condition, prior therapy, and the patient's clinical history and response to the therapeutic agent. The proper dose can be adjusted according to the judgment of the attending physician such that it can be administered to the patient one time or over a series of administrations. The pharmaceutical composition can be administered as a sole therapeutic or in combination with additional therapies as needed.

If the pharmaceutical composition has been lyophilized, the lyophilized material is first reconstituted in an appropriate liquid prior to administration. The lyophilized material may be reconstituted in, e.g. bacteriostatic water for injection (BWFI), physiological saline, phosphate buffered saline (PBS), or the same formulation the modified microorganism had been in prior to lyophilization. However, a modified microorganism of the present invention (e.g. lyophilized bacteria) could also be encapsulated and directly administered orally (e.g. without being reconstituted).

Pharmaceutical compositions for injection may be presented in unit dosage form, e.g. in ampoules or in multi-dose containers, with an added preservative. In addition, several recent drug delivery approaches have been developed and the pharmaceutical compositions of the present invention are suitable for administration using these new methods, e. g. Inject-ease, Genject, injector pens, and needleless devices such as MediJector and BioJector. The present pharmaceutical composition can also be adapted for yet to be discovered administration methods. [Langer, 1990, Science, 249: 1527-1533].

The pharmaceutical composition can also be formulated as a depot preparation. Such long acting formulations may be administered by implantation (for example subcutaneously, into the ligament or tendon, subsynovially or intramuscularly), by subsynovial injection or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

The pharmaceutical compositions may also be in a variety of conventional depot forms employed for administration to provide reactive compositions. These include, for example, solid, semi-solid and liquid dosage forms, such as liquid solutions or suspensions, slurries, gels, creams, balms, emulsions, lotions, powders, sprays, foams, pastes, ointments, salves, balms and drops.

The pharmaceutical compositions may, if desired, be presented in a vial, pack or dispenser device which may contain one or more units dosage forms containing the active ingredient. In one embodiment, the dispenser device can comprise a syringe having a single dose of the liquid formulation ready for injection. The syringe can be accompanied by instructions for administration.

The pharmaceutical composition may further comprise additional pharmaceutically acceptable components. Other pharmaceutically acceptable carriers, excipients, or stabilizers, such as those described in Remington's Pharmaceutical Sciences 16th edition, Osol, A. Ed. (1980) may also be included in formulations described herein, provided that they do not adversely affect the desired characteristics of the formulation. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g. Zn-protein complexes); biodegradable polymers, such as polyesters; salt-forming counterions, such as sodium, polyhydric sugar alcohols; amino acids, such as alanine, glycine, asparagine, 2-phenylalanine and threonine; sugars or sugar alcohols, such as lactitol, stachyose, mannose, sorbose, xylose, ribose, ribitol, myoinisitose, myoinisitol, galactose, galactitol, glycerol, cyclitols (e.g. inositol), polyethylene glycol; sulfur containing reducing agents, such as glutathione, thioctic acid, sodium thioglycolate, thioglycerol, [alpha]-monothioglycerol, and sodium thio sulfate; low molecular weight proteins, such as human serum albumin, bovine serum albumin, gelatine, or other immunoglobulins; and hydrophilic polymers, such as polyvinylpyrrolidone.

The formulations described herein are useful as pharmaceutical compositions in the treatment and/or prevention of the pathological medical condition as described herein in a subject (e.g. a patient in need thereof, e.g. in a healthy subject for preventative purposes). The term “treatment” when used in the context of a method for treatment refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The compositions of the invention can be formulated as neutral or salt forms.

Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

As used herein, the term “treating” and “treatment” when used in the context of a method for treatment refers to administering to a subject a therapeutically effective amount of a pharmaceutical composition according to the invention. A “therapeutically effective amount” may refer to an amount of the pharmaceutical composition which is sufficient to treat or ameliorate a disease or disorder, to delay the onset of a disease or to provide any therapeutical benefit in the treatment or management of a disease.

As used herein, the term “prophylaxis” refers to the use of an agent for the prevention of the onset of a disease, disorder, syndrome or condition. A “prophylactically effective amount” defines an amount of the active component or pharmaceutical agent sufficient to prevent the onset or recurrence of a disease or to prevent or alleviate symptoms and/or complications related to the disease.

Those “in need of treatment” include those already with the disorder, as well as those in which the disorder is to be prevented.

The term “disease” is intended to be selected from the group consisting of: infectious disease, gastrointestinal disorder, inflammatory bowel disease, Celiac Disease, cancer, gastrointestinal cancer, Gastrointestinal adenocarcinoma, inflammatory disease, auto-immune disease, poisoning, foodborne poisoning, allergy, parasitic disease, intestinal infectious diseases (e.g. Cholera due to Vibrio cholerae 01, biovar cholerae; Cholera due to Vibrio cholerae 01, biovar eltor; Cholera); Typhoid and paratyphoid fevers; Infection due to Salmonella typhi; Paratyphoid fever A; Paratyphoid fever B; Paratyphoid fever C; Paratyphoid fever; Infection due to Salmonella paratyphi; Salmonella infections; infection or foodborne intoxication due to any Salmonella species other than S. typhi and S. paratyphi; Salmonella enteritis; Salmonellosis; Salmonella sepsis; Localized salmonella infections; Salmonella arthritis; Salmonella meningitis; Salmonella osteomyelitis; Salmonella pneumonia; Salmonella renal tubulo-interstitial disease; Salmonella infection; Shigellosis; Shigellosis due to Shigella dysenteriae; Group A shigellosis; Shiga-Kruse dysentery; Shigellosis due to Shigella flexneri; Group B shigellosis; Shigellosis due to Shigella boydii; Group C shigellosis; Shigellosis due to Shigella sonnei; Group D shigellosis; Bacillary dysentery; bacterial intestinal infections; Enteropathogenic Escherichia coli infection; Enterotoxigenic Escherichia coli infection; Enteroinvasive Escherichia coli infection; Enterohaemorrhagic Escherichia coli infection; intestinal Escherichia coli infections; Escherichia coli enteritis; Campylobacter enteritis; Enteritis due to Yersinia enterocolitica; Enterocolitis due to Clostridium difficile; Foodborne intoxication by Clostridium difficile; Pseudomembranous colitis; Bacterial enteritis; Foodborne staphylococcal intoxication; Botulism; foodborne intoxication due to Clostridium botulinum; Foodborne Clostridium perfringens intoxication; Foodborne Clostridium welchii intoxication; Enteritis necroticans; Pig-bel; Foodborne Vibrio parahaemolyticus intoxication; Foodborne Bacillus cereus intoxication; Bacterial foodborne intoxication; Amoebiasis; infection due to Entamoeba histolytica; Acute amoebic dysentery; Acute amoebiasis; Intestinal amoebiasis; Chronic intestinal amoebiasis; Amoebic nondysenteric colitis; Amoeboma of intestine; Amoeboma; Amoebic liver abscess; Hepatic amoebiasis; Amoebic lung abscess; Amoebic abscess of lung (and liver); Amoebic brain abscess; Amoebic abscess of brain (and liver)(and lung); Cutaneous amoebiasis; Amoebic appendicitis; Amoebic balanitis; Amoebiasis; protozoal intestinal diseases; Balantidiasis; Balantidial dysentery; Giardiasis; Iambliasis; Cryptosporidiosis; Isosporiasis; Infection due to Isospora belli and Isospora hominis; Intestinal coccidiosis; Isosporosis; Intestinal trichomoniasis; Sarcocystosis; Sarcosporidiosis; Flagellate diarrhoea; Protozoal colitis, Protozoal diarrhea; Protozoal dysentery; Viral intestinal infections; Rotaviral enteritis; Acute gastroenteropathy due to Norwalk agent; Small round structured virus enteritis; Adenoviral enteritis; viral enteritis; Viral intestinal infection; Viral enteritis; Viral gastroenteritis; Viral gastroenteropathy; gastroenteritis and colitis of infectious origin; Catarrh, enteric or intestinal; Diarrhoea acute bloody; Diarrhoea acute haemorrhagic; Diarrhoea acute watery; Diarrhoea dysenteric; Diarrhoea epidemic; Infectious or septic colitis, enteritis or gastroenteritis; Neonatal diarrhoea.

When used herein, the application of an “antibiotic” in the method for producing or isolating a modified microorganism refers to another chemical pre-treatment such as pretreatment with trichloroacetic acid (TCA). It may refer, but is not limited to mupirocin (inhibits protein synthesis), mutanolysin (inhibits membrane structure), myriocin (inhibits sphingolipid synthesis), cefalotin (inhibits membrane structure).

When used herein, the application of a “detergent” in the method for producing or isolating a modified microorganism also refers to another chemical pre-treatment such as pretreatment with trichloroacetic acid (TCA) and may lead to modification(s) such as depletion or modification of LTA or WTA.

When “trichloroacetic acid” is used as a chemical pretreatment in the method for producing or isolating said modified microorganism as defined elsewhere herein at least 2%, at least 4%, at least 6%, at least 8%, such as 2%, 3%, 4%, 5%, 6%, 7%, 8%, 9% 10% or even more trichloroacetic acid is applied. Preferably, when trichloroacetic acid is used as a chemical pretreatment in the method for producing or isolating said modified microorganism as defined elsewhere herein 10% trichloroacetic acid (e.g. in water, PBS or DPBS) is applied. Such chemical pre-treatment is preferred herein and ensures a stable association between the microorganism and the lipid carrier as it is described elsewhere herein and described in the Example section.

The stable association of the lipid carrier with the exterior surface of said cell of said modified microorganism may also be achieved by adapting the culture conditions. Preferably, MRS-medium as defined elsewhere herein is used as a culture medium and ABM medium is used as a loading medium, preferably for Lactobacillus paracasei L3 (see Table 2 and 3). Most preferably, the culture conditions comprise inoculating in MRS-medium and cultivating 0/N followed by a 5% inoculation of the culture in fresh MRS-Medium and cultivation 0/N, followed by loading in ABM-Medium added with 1% cholesterol for 3 days. The term “ABM medium” (ACARYON Bifido Medium) when used in the context of the method for producing, isolating or loading said microorganism refers to SBSM medium, which contains casein peptone (12 g/l), meat peptone (5 g/l), sodium chloride (5 g/l), beef extract (3 g/l), yeast extract (3 g/l), cornstarch (1 g/l), glucose (2.5 g/l), lactulose (2.5 g/l), cysteine-hydrochloride (0.5 g/l), riboflavin (0.01 g/l) and propionic acid (99%; 5 ml/l), however instead of meat peptone, soya peptone (5 g/l) is used in ABM. As mentioned above, when said particular ABM medium is used for loading, also 1% cholesterol is preferably added to said ABM medium (see Table 2 and 3).

Other culture conditions that may help to ensure stability of the association include cultivating cells in presence of lipase or an inhibitor of lipid synthesis in the growth and/or loading medium as described elsewhere herein.

The term “lipase” refers to, but is not limited to any class of enzymes that break down fats, produced by the liver, pancreas, and other digestive organs or by certain plants. Any lipase known to a person skilled in the art may be applicable herein.

The term “inhibitor of lipid synthesis” refers to, but is not limited to agents that affect a cell's ability to synthesize lipids. Lipids are important in maintaining the structural integrity of the cell as they are the main component of the cell membrane. Any inhibitor of lipid synthesis known to a person skilled in the art may be applicable herein.

The term “subject” is intended to include living organisms. Examples of subjects include mammals, e.g. humans, dogs, cows, horses, pigs, sheep, goats, cats, mice, rabbits, rats, and transgenic non-human animals. In preferred embodiments of the invention, the subject is a human (e.g. patient).

As used herein, the term “mammalian” may refer to a mammal organism, e.g. such as human or an animal, such as a dog, cat, cattle, pig, horse, camel, sheep, mouse, rat, poultry, fish preferably human.

As used herein, “about” means especially+/−10%, +/−5% or +/−3% (referring to the given numeric value), if not indicated otherwise.

Infectious Diseases:

For a number of infectious diseases, effective vaccines are missing, and the increasing rate of drug resistances is complicating the use of conventional antimicrobial therapy. Due to this there is a need for novel therapeutic and prophylactic approaches against infectious diseases, in particular enteric infectious diseases which continue to cause massive morbidity and mortality in humans. Effective vaccines are still not available for a number of important diarrheal diseases, and, as mentioned, controlling these with conventional antimicrobial therapy is being complicated by increasing rates of drug resistance.

The initial and critical step that leads to an infection is the binding of a pathogen or its toxin(s) to host cells. Anti-adhesion strategies aim to prevent and/or displace said binding and to prevent or treat the subsequent infection and/or its symptoms. Anti-adhesion strategies have attracted increasing interest as a source of novel therapeutics to prevent and treat infectious diseases. An advantage of such approaches is that the pathogen is not killed. As a consequence, anti-adhesion strategies may avoid problems associated with release of toxic products from dead bacteria and they may put much less selection pressure on pathogens, reducing the risk of resistance development.

Several anti-adhesion approaches may be envisaged, including providing receptor analogs or adhesin analogs, inhibition of adhesins and their host receptors, vaccination with adhesins or analogs, or inhibiting the synthesis of adhesins or their host receptor. Although the initial adhesion of pathogens or their toxins to host cells may happen through protein-protein interactions or phospholipid-protein interactions it is often mediated by protein-carbohydrate interactions. As a consequence, many carbohydrates or carbohydrate mimicking substances have been developed based either on proteins, polymers, calixarenes, dendrimers, cyclodextrins, cyclopeptides, fullerenes, gold nanoparticles and quantum dots. In order to efficiently block protein-carbohydrate interactions synthetic neutralization agents need to comprise multiple oligosaccharide epitopes displayed on complex three-dimensional scaffolds, conditions that may be difficult to reproduce synthetically. However, to date, clinical results in human were disappointing, mostly because of toxicity or lack of efficacy [e.g. Synsorb PK (Synsorb Biotech); Tolevamer (Genzyme)].

Accordingly, U.S. Pat. No. 6,833,130 discloses recombinant microorganisms, genetically modified to express carbohydrate structures that mimic the natural binding moieties of bacterial toxins. Such microorganisms are able to present the binding moiety at high density. The efficacy of these microorganisms in binding toxins and protecting animals in lethal challenge models has been demonstrated. However, in order to use microorganisms in humans and animals, it is not only necessary for these microorganisms to express a binding moiety for a pathogenic ligand at sufficient density, but it should preferably be harmless, ideally it should be non-pathogenic and not genetically modified or recombinant.

There is some prior art that reports the ability of non-pathogenic microorganisms to specifically co-aggregate other microorganisms. However, the detailed mechanism of interaction is unknown. A few pathogenic microorganisms were described to naturally express structures that may mimic natural binding moiety for pathogenic ligands. Some publications further report the possibility to produce nanoparticles decoys with toxin receptor to bind the toxin. The potential of this approach is supported through in vitro and in vivo studies. However, the use of nanoparticles in the gastrointestinal tract may be limited by natural biodegradation, absorption and their potential intrinsic toxic effect on epithelial cells, on the gut microbiota or on extra-intestinal organs where they may accumulate. Those potential toxic effects may be further amplified if the nanoparticles are decoyed with a pathogen or a toxin. Furthermore, the efficacy of nanoparticles may be limited by many factors including susceptibility to gastrointestinal environment (enzymes, pH etc.) or the inability of some nanoparticles to cross the mucus layer and to locate near the epithelium where pathogen microorganisms may locally release their toxin. Thus, as is evident from the above, the prior art provides recombinant microorganisms for use in treating infectious disease, particularly enteric infectious disease which may be harmful, and which are not be food grade organisms. Alternatively, the prior art provides agents which have shown to be toxic when administered to mammals and for which toxicity potential is still largely unknown.

Currently, no non-pathogenic microorganisms capable of binding a lipid-carrier (i.e. glycolipid or a lipopeptide) whose carried moiety (i.e. carbohydrate or peptide) may be able to bind a toxin, the receptor for a toxin, a pathogen or the receptor for a pathogen have been described. Furthermore, no non-pathogenic microorganisms capable of binding a fusion-protein composed of an anchor-domain and a peptide that is able to bind a toxin, the receptor for a toxin, a pathogen or the receptor for a pathogen have been described. However, it is highly desirable to block the interaction between pathogenic ligands such as toxins from pathogenic microorganisms or the pathogen microorganism itself and their cognate receptors on the surface of mammalian cells, since this interaction is crucial for disease pathogenesis. Such a strategy is particularly promising for fighting against enteric infectious diseases which cause a high morbidity and mortality in humans, since no effective vaccines are available and rates of drug resistance against conventional antibiotic therapies increase. Due to this there is a need for novel therapeutic and prophylactic approaches against infectious diseases, in particular enteric infectious diseases. It, thus, follows that the technical problem underlying the present invention is to comply with the needs described above. The solution to this technical problem is achieved by providing the embodiments characterized herein, exemplified in the appended examples and set out in the claims.

Gastrointestinal Disorders and Gastrointestinal Cancers:

Disorders include acute, chronic, recurrent or functional disorders while covering a broad range of diseases, including the most common acute and chronic inflammatory bowel disease, Celiac Disease etc. For most of these disorders, no specific treatment is available.

The primary therapy is mostly relying on immune suppressor that may have significant side effects. Other existing approaches are based on the microbiota. Thus, in severe cases, feces transplantation was successfully used.

Another approach that attracted attention is the use of non-pathogenic lactic acid bacteria with anti-inflammatory properties. Such microorganisms may be taken chronically without risk of side effects. However, this approach is still confronted to several limits.

Thus, if the anti-inflammatory properties are introduced through genetic modifications, risks related to the chronic intake of recombinant microorganisms may significantly limit their use. A more appealing approach is the use of strains with natural anti-inflammatory properties. However, the gut contains an amazing number of microorganisms (up to 1×10¹⁰ per gram feces). Thus, the potential anti-inflammatory effect of exogenously administered bacteria is strongly diluted. Gastrointestinal adenocarcinoma is a common malignant disease worldwide. Non-Pathogenic microorganisms, especially Lactic-Acid Bacteria (LAB) have been reported to have anticancer properties like antioxidant activity, inhibition of tumor cell proliferation or induction of apoptosis. However, the use of non-pathogenic microorganisms may be confronted to the same limits as those mentioned above.

The beneficial effect of non-pathogenic with beneficial properties (anti-inflammatory, anti-cancer etc.) would be much stronger if such microorganisms would be able to specifically target the site of disease (site of inflammation or the tumor). Thus, there is a need for a new strategy that may help to target the non-pathogenic microorganisms with beneficial properties at the sites of need without genetic modifications. Along the intestinal tract and depending on the physiological or pathological conditions endothelial cells may express different carbohydrate structures or carbohydrate receptors. As example, under inflammation conditions (as it is the case in IBD), cells are known to express new and specific carbohydrate structures. Furthermore, cancer cells are known to express new carbohydrate antigens not present on the surface of healthy cells.

Currently, only GMO approaches or non-GMO method using peptide fused to a protein-domains know to anchor themselves to the surface of suitable non-pathogenic microorganisms are available to display specific ligand or receptor on the surface of non-pathogenic microorganisms. Thus, there is a high need for new non-GMO methods that would allow displaying a receptor or a ligand (especially when the said receptor or ligand is a carbohydrate structure) on the surface of microorganisms with beneficial properties, thereby enabling the said microorganisms to target the site where their beneficial properties are needed.

Mucosal Immunization:

The use of non-pathogenic bacteria like LAB to induce antigen specific immune responses is a field that is attracting some attention. Such approaches may be used to develop vaccines against pathogens, vaccines against cancer or allergy vaccines (that induce tolerance against the antigen). Again, to induce a non-pathogenic microorganism to display an antigen on its surface the most used approach is the genetic modification of the said non-pathogenic microorganisms. Beside the fact that this may result in the release of live recombinant microorganisms into nature, significant safety concerns have been raised that may limit the application of this technology including i) transfer of material to other microorganisms, ii) integration of the plasmid into the genome of the recipient or iii) integration of the DNA into host genome.

Alternatively, non-GMO approaches are using antigens fused to protein-domains know to anchor itself to the surface of suitable non-pathogenic microorganisms. However, these applications of these approaches may be limited due to concerns already mentioned above. Also, many pathogenic, cancer or allergy antigens may be carbohydrates. The ability of the mentioned approaches to present carbohydrate antigen is still completely unknown. Thus, there is a high need for a new technology that allows to equip non-pathogenic bacteria with pathogenic, cancer or allergy antigens, especially carbohydrate antigens without using genetic modifications.

### EMBODIMENTS

In some aspects, the present invention relates to a modified microorganism comprising: a cell and a heterologous lipid and/or protein carrier (e.g. said heterologous lipid and/or protein carrier is not expressed or synthetized by said microorganism, e.g. comprising a LysM-based protein carrier as described in Ganesh et al., 2014, Schmidt et al., 2011), said lipid and/or protein carrier comprising: a) a lipid portion, wherein said lipid portion is at least partially associated with an exterior surface of said cell of said modified microorganism, wherein said lipid portion comprising: a ceramide-like glycolipid moiety (and/or a ceramide moiety and/or a sphingolipid moiety and/or sphingosine moiety) and/or a fatty acid moiety; preferably said exterior surface of said cell comprising: a cell wall and/or a cell membrane and/or an outer cell membrane and/or a polysaccharide (e.g. capsule polysaccharide); further preferably said lipid portion is at least partially incorporated and/or adhered and/or bound to said exterior surface of said cell; even more preferably said lipid portion further comprises an amino alcohol moiety, preferably the amino alcohol moiety is sphingosine and b) a non-lipid portion, wherein said microorganism is capable of locating and/or displaying said non-lipid portion or fragment thereof onto the exterior surface of said cell, preferably said non-lipid portion comprising a carbohydrate moiety, a lipopeptide moiety (e.g. a glycosylated lipopeptide moiety, e.g. a carbohydrate antigen) or a peptide moiety, wherein preferably said carbohydrate moiety of said non-lipid portion is a sialic acid residue; further preferably said non-lipid portion is capable of binding and/or reducing toxicity and/or neutralizing a toxin; optionally, said modified microorganism (e.g. said lipid carrier) further comprising a heterologous steroid moiety; preferably said steroid moiety is cholesterol, a derivative or analog thereof.

In some aspects of the present invention, a microorganism may already comprise an endogenous lipid carried (e.g. identical or non-identical with the heterologous lipid carrier of the present invention as described herein, e.g. a glycolipid, etc.), wherein: (a) the concentration of said endogenous lipid carrier is increased with said heterologous lipid carrier (e.g. via methods and/or uses as described herein, e.g. loading protocol/s as described, e.g. in the examples section, e.g. Table 1); or (b) said heterologous and endogenous lipid carries are non-identical and are both comprised in the modified microorganism of the present invention (e.g. a microorganism may already comprise an endogenous GM1 saccharide moiety on its surface (e.g. not necessary a whole GM1 molecule as described herein) and is then modified by the means of adding a heterologous lipid carrier (e.g. GM1 molecule).

The present invention also relates to a composition comprising one or more of the modified microorganisms of the present invention; preferably said composition comprising a mixture of same (e.g. identical, same species, same strain) or different (e.g. non-identical, different species, different strains) modified microorganisms.

In some aspects, the present invention relates to the composition of the present invention, wherein said composition is a pharmaceutical, diagnostic, probiotic, prebiotic composition or a food composition.

In some aspects, the present invention relates to the composition of the present invention, wherein said composition further comprises a pharmaceutical carrier.

In some aspects, the present invention relates to the composition of the present invention, wherein said composition is suitable for oral, enteral, dermal, topical, urogenital, inhalational administration, preferably suitable for oral or enteral administration.

Additionally, the present invention also relates to a vaccine or adjuvant comprising the microorganism or composition of the present invention (e.g. said microorganism is pathogenic and/or immunogenic, e.g. wherein said microorganism is alive and/or attenuated and/or pasteurized and/or inactivated and/or non-living and/or dead), preferably said vaccine or adjuvant is suitable for oral or enteral administration.

The present invention also comprises a method for treatment, amelioration, prophylaxis or diagnostics of a disease, said method comprising:
i) providing one or more (e.g. mixtures) microorganism, composition, vaccine or adjuvant as defined elsewhere herein to a subject in need thereof (e.g. human or animal) as defined elsewhere herein;
ii) administering a therapeutically effective amount of said microorganism, composition, vaccine or adjuvant to said subject as described herein.

The present invention also comprises a method for eliciting or modulating an immune response, said method comprising:
i) providing the microorganism, composition, vaccine or adjuvant as defined elsewhere herein to a subject (e.g. patient) (e.g. human or animal);
ii) administering a therapeutically effective amount of said microorganism, composition, vaccine or adjuvant to said subject (e.g. patient).

The present invention also comprises the microorganism, composition, vaccine or adjuvant as defined elsewhere herein for use as a medicament.

The present invention also comprises the microorganism, composition, vaccine or adjuvants defined elsewhere herein, for use in one or more of the following methods:
i) in a method for treatment, amelioration, prophylaxis or diagnostics of a disease (e.g. a bacterial, viral, or fungal infection or autoimmune diseases, e.g. suitable for topical applications to skin and/or other sites such urogenital tract, respiratory system or oral cavity);
ii) in a method for modulating the mammalian immune response;
iii) in a method for eliciting or modulating an immune response;
iv) in a method for monitoring development of a disease and/or assessing the efficacy of a therapy of a disease;
v) in a method for screening a candidate compound for activity against a disease;
vi) in a method for delivering a pharmaceutically active compound, preferably said delivering is to a mucosal tissue of a subject (e.g. patient);
vii) in a method for vaccine delivery;
viii) in a method for screening of microorganisms capable of binding the lipid carrier according to any one of preceding items;
ix) in a method for binding and/or reducing toxicity and/or neutralizing a toxin;
x) in a method for binding a receptor of a toxin;
xi) in a method for binding and/or reducing the pathogenicity and/or neutralizing a pathogenic microorganism;
xii) in a method for binding a receptor of a pathogenic microorganism;
xiii) in a method for producing or isolating the microorganism as defined herein.

The present invention also comprises a use of the microorganism, composition, vaccine or adjuvant as defined elsewhere herein for one or more of the following:
i) for treatment, amelioration, prophylaxis or diagnostics of a disease (e.g. a bacterial, viral, or fungal infection or autoimmune diseases, e.g. suitable for topical applications to skin and/or other sites such urogenital tract, respiratory system or oral cavity);
ii) for modulating the mammalian immune response;
iii) for eliciting or modulating an immune response;
iv) for monitoring development of a disease and/or assessing the efficacy of a therapy of a disease;
v) for screening a candidate compound for activity against a disease;
vi) for delivering a pharmaceutically active compound, preferably said delivering is to a mucosal tissue of a subject (e.g. patient);
vii) for vaccine delivery;
viii) for screening of microorganisms capable of binding the lipid carrier according to any one of preceding items;
ix) for binding and/or reducing toxicity of and/or neutralizing a toxin;
x) for binding a receptor of a toxin;
xi) for binding and/or reducing the pathogenicity and/or neutralizing a pathogenic microorganism;
xii) for binding a receptor of a pathogenic microorganism;
xiii) for producing or isolating the microorganism as defined elsewhere herein;

The present invention also comprises a kit comprising the microorganism, composition, vaccine or adjuvant as defined elsewhere herein.

The present invention also comprises a kit for performing a method as defined elsewhere herein.

In some aspects, the present invention relates to the modified microorganism, composition, vaccine, adjuvant, method or use of the present invention, wherein said modified microorganism has one or more of the following characteristics: said cell of said microorganism comprising a cell membrane (e.g. separating the cytoplasm of said cell from the exterior of the cell), preferably said cell membrane further comprising a peptidoglycan; said cell of said microorganism comprising a cell wall, preferably said cell wall comprising a peptidoglycan; said cell of said microorganism comprising a cell membrane, wherein said cell membrane further comprising a peptidoglycan; said cell of said microorganism is not comprising an outer membrane, (e.g. separating the prokaryotic periplasm from the exterior of said cell of said microorganism), preferably said microorganism is a gram-positive bacterium; said cell of said microorganism comprising an outer membrane (e.g. separating the prokaryotic periplasm from the exterior of said cell of said microorganism), preferably said microorganism is a gram-negative bacterium; said cell of said microorganism comprising a cell wall, preferably said cell wall comprises mannoproteins (e.g. yeast); said cell of said microorganism is not comprising a recombinant and/or fusion polypeptide, preferably said recombinant and/or fusion polypeptide is obtained by the means of artificial genetic manipulation; said microorganism is non-pathogenic.

In some aspects, the present invention relates to the modified microorganism, composition, vaccine, adjuvant, method or use of the present invention, wherein said lipid carrier is selected from the group consisting of: Monosialotetrahexosylganglioside (GM1), or Monosialotetrahexosylganglioside red (GM1red) as defined elsewhere herein, Globotriaosylceramide (Gb3), a GM1-Gb3 chimera as defined elsewhere herein, Ganglioside GD1a, Gangliosides GM2, GD2, GD1b, GT1b, GT1c, GQ1c, GA1, GM1b, Gangliosides GM3, GD3 and GT3, Gangliosides Gb4, Blood Group Type I, Type 2, Blood Group A, Blood Group B, Blood Group H, Blood Group H Type 1, Blood Group H Type 2, Blood Group H Type 3, Lewis y, Lewis a, Lewis b, Lewis x, H, Sialyl Lewis x, Sialyl Lewis a, Sialyl Lewis b, Sialyl Lewis x, alpha Gal epitope, Gal a1-3Galß1-4GlacNAc, and derivatives and analogs thereof.

Monosialotetrahexosylganglioside red (GM1-red; reductive ozonized GM1) is a derivative of the lipid carrier GM1 that is deprived from a sphingosine moiety. N-Hexadecanyl-Ceramide-trihexoide and N-Octadecanyl-Ceramide-trihexoide are derivatives of the lipid carrier Gb3 that differ in the length of their fatty acid and may also be applied in the present invention (see the Examples). N-(1-Adamantaneacetyl)-ceramide trihexoside is also a derivative of the lipid carrier Gb3 for which one sphingosine is replaced with an Adamantaneacetyl. Such derivative may also be applied by the present invention (see the Examples). Preferably, according to the present invention said lipid carrier is Monosialotetrahexosylganglioside (GM1), derivatives thereof, or a GM1-Gb3 chimera as defined elsewhere herein. Also preferred according to the present invention is said lipid carrier being GM1, a derivative thereof, or comprising the carbohydrate structure of GM1 which function as a core structure. Meaning GM1 is linked directly or via linker(s) to one or more moieties such as carbohydrate(s), peptide(s) or protein(s). Alternatively, the carbohydrate structure of GM1 is linked directly or via linker(s) to the lipid portion of any lipid carrier as defined elsewhere herein that further is linked directly or via linker(s) to one or more moieties such as carbohydrate(s), peptide(s) or protein(s) or the carbohydrate structure of GM1 is linked directly or via linker(s) to one or more moieties such as carbohydrate(s), peptide(s) or protein(s) that further is (are) linked directly or via linker(s) to the lipid portion of any lipid carrier as defined elsewhere herein.

In some aspects, the present invention relates to the modified microorganism, composition, vaccine, adjuvant, method or use of the present invention, wherein said microorganism is naturally-occurring (e.g. non-genetically modified, e.g. not obtained by means of artificial genetic manipulation), preferably said naturally-occurring microorganism is obtainable from one or more of the following sources: microflora of a vertebral organism (e.g. mammalian, avian (e.g. poultry), bovine, porcine, ovine, caprine, leporine, or piscine organism), preferably microflora of a digestive or urinary system (e.g. microflora of a gastrointestinal tract (GIT) or an urinary tract) or skin microflora of said vertebral organism or microflora of the respiratory tract; further preferably said naturally-occurring microorganism is obtainable from feces or urine or sputum of said vertebral organism; most preferably said mammalian organism is human; and soil microbiota; and microbiota of water such as sea or fresh water; microbiota of plants or other natural source/s or environment/s and microbiota from food.

In some aspects, the present invention relates to the modified microorganism, composition, vaccine, adjuvant, method or use of the present invention, wherein said microorganism is selected from the group consisting of: a) a bacterium (e.g. gram-positive or gram-negative (e.g. from genus Bacteroides, e.g. Bacteroides vulgatus) bacterium; preferably said bacterium is a gram-positive bacterium; further preferably said gram-positive bacterium is selected from the genera consisting of: Lactobacillus (e.g. Lactobacillus paracasei, Lactobacillus rhamnosus, Lactobacillus intestinalis, Lactobacillus murinus, Lactobacillus reuteri, Lactobacillus acidophilus, Lactobacillus crispatus, Lactobacillus fermentum, Lactobacillus gasseri, Lactobacillus salivarius, Lactobacillus brevis, Lactobacillus plantarum, Lactobacillus casei, Lactobacillus acidophilus or Lactobacillus vaginalis), Bifidobacterium (e.g. Bifidobacterium animalis, Bifidobacterium breves Bifidobacterium adolescentis, Bifidobacterium longum, Bifidobacterium intestinalis, Bifidobacterium lactis, Bifidobacterium bacteroides vulgatus, Bifidobacterium xylanisolvens, Bifidobacterium infantis or Bifidobacterium pseudocatenulatum), Clostridium (e.g. Clostridium perfringens, Clostridium coccides, Clostridium xylaniticus), Enterococcus (e.g. Enterococcus avium, Enterococcus faecalis, Enterococcus faecium, Enterococcus gallinarum, Enterococcus casseliflavus or Enterococcus sp.), Pediococcus (e.g. Pediococcus pentosaceus) and Streptococcus (e.g. Streptococcus salivarius or Streptococcus vestibularis); most preferably said gram-positive bacterium is selected from the group consisting of: Lactobacillus paracasei, Lactobacillus reuteri; or said bacterium is a non-pathogenic and/or opportunistic pathogen; b) a fungus; preferably said fungus is selected from the group consisting of: Candida yeasts, Saccharomyces yeasts and yeasts in the family Dipodascaceae; further preferably said Dipodascaceae yeasts are Galactomyces, Geotrichum or Saprochaete yeasts, most preferably said Saccharomyces yeast is Saccharomyces boulardii, S. cerevisiae, S. pastorianus, or Schizosaccharomyces pombe, or c) a protozoa, said protozoa being non-pathogenic to human, preferably said protozoa is Chilomastix mesnili, Endolimax nana, Entamoeba coli, Entamoeba dispar, Entamoeba hartmanni, Entamoeba polecki or Iodamoeba buetschlii.

In some aspects, when the modified microorganism according to the present invention is an opportunistic pathogen, an opportunistic pathogen is selected from the group consisting of genera: Abiotrophia Acanthopleuribacter Acaricomes Acetanaerobacterium Acetatifactor Acetitomaculum Acetivibrio Acetoanaerobium Acetobacter Acetobacterium Acetofilamentum Acetogenium Thermoanaerobacter Acetohalobium Acetomicrobium Acetonema Acetothermus Acholeplasma Achromatium Achromobacter Acidaminobacter Acidaminococcus Acidianus Acidicaldus Acidicapsa Acidiferrobacter Acidilobus Acidimicrobium Acidiphilium Acidiplasma Acidisoma Acidisphaera Aciditerrimonas Acidithiobacillus Acidobacterium Acidocella Acidomonas Acidothermus Acidovorax Acinetobacter Acrocarpospora Actibacter Actibacterium Actinaurispora Plantactinospora Actinoallomurus Actinoalloteichus Actinobacillus Actinobaculum Actinobispora—synonym: Pseudonocardia Actinocatenispora Actinocorallia Actinokineospora Actinomadura Actinomyces Actinomycetospora Actinophytocola Actinoplanes Actinopolymorpha Actinopolyspora Actinopycnidium—synonym: Streptomyces Actinospica Actinosporangium Streptomyces Actinosynnema Actinotalea Adhaeribacter Adlercreutzia Advenella Aegyptianella Aequorivita Aeribacillus Aeriscardovia Aerococcus Aeromicrobium Aeromonas Aeropyrum Aestuariibacter Aestuariibaculum Aestuariicola Lutimonas Aestuariihabitans Aestuariimicrobium Aestuariispira Afifella Afipia Agaricicola Agarivorans Aggregatibacter Agitococcus Agreia Agrobacterium Agrococcus Agromonas Bradyrhizobium Agromyces Ahrensia Aidingimonas Akkermansia Albibacter Albidiferax Albidovulum Albimonas Alcaligenes Alcanivorax Algibacter Algicola Algimonas Algiphilus Algisphaera Algoriphagus Aliagarivorans Alicycliphilus Alicyclobacillus Aliicoccus Aliidiomarina Aliifodinibius Aliiglaciecola Aliivibrio Alishewanella Alistipes Alkalibacillus Alkalibacter Alkalibacterium Alkalibaculum Alkaliflexus Alkalilimnicola Alkalimonas Alkaliphilus Alkalispirillum Alkalitalea Alkanibacter Alkanindiges Allisonella Alloactinosynnema Allobacillus Allobaculum Allocatelliglobosispora Allochromatium Allofustis Alloiococcus Allokutzneria Allomonas Allonocardiopsis Alloprevotella Allorhizobium Rhizobium Alloscardovia Alpinimonas Alsobacter Altererythrobacter Alteribacillus Alterococcus Alteromonas Alysiella Amantichitinum Amaricoccus Ameyamaea Aminiphilus Aminivibrio Aminobacter Aminobacterium Aminomonas Ammonifex Ammoniphilus Amnibacterium Amoebobacter Amorphosporangium Actinoplanes Amorphus Amphibacillus Amphritea Ampullariella Actinoplanes Amycolata Pseudonocardia Amycolatopsis Amycolicicoccus Anaeroarcus Anaerobacillus Anaerobacter Anaerobacterium Anaerobaculum Anaerobiospirillum Anaerobranca Anaerocella Anaerococcus Anaerofilum Anaerofustis Anaeroglobus Anaerolinea Anaeromusa Anaeromyxobacter Anaerophaga Anaeroplasma Anaerorhabdus Anaerosalibacter Anaerosinus Anaerosphaera Anaerosporobacter Anaerostipes Anaerotruncus Anaerovibrio Anaerovirgula Anaerovorax Anaplasma Ancalochloris Ancalomicrobium Ancylobacter Anderseniella Andreprevotia Aneurinibacillus Angiococcus Angulomicrobium Angustibacter Anoxybacillus Anoxynatronum Antarcticimonas Antarctobacter Aquabacter Aquabacterium Aquamicrobium Aquaspirillum Aquibacter Aquicella Aquifex Aquiflexum Aquihabitans Aquimarina Aquimonas Aquincola Aquipuribacter Aquisalibacillus Aquisalimonas Aquisphaera Aquitalea Arachnia Propionibacterium Arcanobacterium Archaeoglobus Archangium Arcicella Arcobacter Arcticibacter Ardenticatena Arenibacter Arenicella Arenimonas Arenitalea Arhodomonas Aridibacter Armatimonas Arsenicicoccus Arsenophonus Arthrobacter Asaccharobacter Asaccharospora Asaia Asanoa Asinibacterium Aspromonas Arenimonas Asteroleplasma Asticcacaulis Atopobacter Atopobium Atopococcus Atopostipes Aurantimonas Auraticoccus Aureibacter Aureicoccus Aureimonas Aureispira Aureitalea Aureivirga Aureobacterium Microbacterium Auritidibacter Austwickia Avibacterium Azoarcus Azohydromonas Azomonas Azomonotrichon Azomonas Azonexus Azorhizobium Azorhizophilus Azospira Azospirillum Azotobacter Azovibrio Bacillus Bacteriolyticum Bacterionema Corynebacterium Bacteriovorax Bacteroides Bactoderma Balnearium Balneatrix Balneimonas Microvirga Balneola Balneomonas Barnesiella Barrientosiimonas Bartonella Basfia Bauldia Bavariicoccus Bdellovibrio Beggiatoa Betjerinckia Belliella Bellilinea Belnapia Beneckea Bergeriella Bergeyella Bermanella Beutenbergia Bhargavaea Bibersteinia Bifidobacterium Bilophila Biostraticola Bisgaardia Bizionia Blastobacter Blastocatella Blastochloris Blastococcus Blastomonas Blastopirellula Blattabacterium Blautia Bogoriella Bordetella Borrelia Bosea Bowmanella Brachybacterium Brachymonas Brachyspira Brackiella Bradyrhizobium Branchiibius Brassicibacter Brenneria Breoghania Brevibacillus Brevibacterium Brevifollis Brevinema Brevundimonas Brochothrix Brockia Brooklawnia Brucella Brumimicrobium Bryantella Marvinbryantia Bryobacter Bryocella Buchnera Budvicia Bulleidia Burkholderia Buttiauxella Butyricicoccus Butyricimonas Butyrivibrio Byssovorax Caedibacter Caenibacterium—synonym: Schlegelella Caenimonas Caenispirillum Caldalkalibacillus Caldanaerobacter Caldanaerobius Caldanaerovirga Calderihabitans Calderobacterium Hydrogenobacter Caldibacillus Caldicellulosiruptor Caldicoprobacter Caldilinea Caldimicrobium Caldimonas Caldisericum Caldisphaera Calditerricola Calditerrivibrio Caldithrix Caldivirga Calidifontibacter Caloramator Caloranaerobacter Caloribacterium Calymmatobacterium Klebsiella Camelimonas Caminibacter Caminicella Campylobacter Candidimonas Canibacter Capnocytophaga Capsularis Prevotella Carbophilus Carboxydibrachium Caldanaerobacter Carboxydocella Carboxydothermus Carboxylicivirga Cardiobacterium Carnimonas Carnobacterium Caryophanon Caseobacter—synonym: Corynebacterium Castellaniella Catalinimonas Catellatospora Catellibacterium Gemmobacter Catellicoccus Catelliglobosispora Catenibacterium Catenococcus Catenovulum Catenulispora Catenuloplanes Catonella Caulobacter Cecembia Cedecea Celeribacter Celerinatantimonas Cellulomonas Cellulophaga Cellulosibacter Cellulosilyticum Cellulosimicrobium Cellvibrio Centipeda Cerasibacillus Cerasicoccus Cesiribacter Cetobacterium Chainia Streptomyces Chelativorans Chelatobacter—synonym: Aminobacter Chelatococcus Chelonobacter Chiayiivirga Chimaereicella Algoriphagus Chitinibacter Chitinilyticum Chitinimonas Chitiniphilus Chitinivorax Chitinophaga Chlamydia Chlamydophila Chlorobaculum Chlorobium Chloroflexus Chloroherpeton Chloronema Chondromyces Christensenella Chromatium Chromatocurvus Chromobacterium Chromohalobacter Chryseobacterium Chryseoglobus Chryseolinea Chryseomicrobium Chryseomonas—synonym: Pseudomonas Chrysiogenes Chthonomonas Chungangia Ciceribacter Citreicella Citreimonas Citricoccus Citrobacter Clavibacter Clevelandina Cloacibacillus Cloacibacterium Clostridiisalibacter Clostridium Cnuella Cobetia Cocleimonas Coenonia Cohaesibacter Cohnella Collimonas Collinsella Colwellia Comamonas Compostimonas Conchiformibius Conexibacter Conglomeromonas Congregibacter Constrictibacter Coprobacillus Coprococcus Coprothermobacter Coraliomargarita Corallibacter Corallococcus Corallomonas Coriobacterium Corynebacterium Cosenzaea Costertonia Couchioplanes Cowdria Ehrlichia Coxiella Crabtreella—synonym: Shinella Craurococcus Crenotalea Crenothrix Cribrihabitans “Crinalium” Cristispira Croceibacter Croceicoccus Croceitalea Crocinitomix Cronobacter Crossiella Cruoricaptor Cryobacterium Cryomorpha Cryptanaerobacter Cryptobacterium Cryptosporangium Cucumibacter Cupriavidus Curtobacterium Curvibacter Cyclobacterium Cycloclasticus Cystobacter Cytophaga Dactylosporangium Daeguia Dasania Dechloromonas Dechlorosoma—synonym: Azospira Deefgea Deferribacter Deferrisoma Defluvibacter Aquamicrobium Defluvicoccus Defluviimonas Defluviitalea Defluviitoga Dehalobacter Dehalococcoides Dehalogenimonas Dehalospirillum Sulfurospirillum Deinobacter Deinococcus Deinobacterium Deinococcus Deleya Delftia Demequina Demetria Dendrosporobacter Denitratisoma Denitrobacterium Denitrovibrio Dermabacter Dermacoccus Dermatophilus Derxia Desemzia Desertibacter Desmospora Desulfacinum Desulfarculus Desulfatibacillum Desulfatiferula Desulfatirhabdium Desulfatitalea Desulfitibacter Desulfitispora Desulfitobacterium Desulfobacca Desulfobacter Desulfobacterium Desulfobacula Desulfobaculum Desulfobotulus Desulfobulbus Desulfocapsa Desulfocella Desulfococcus Desulfoconvexum Desulfocurvus Desulfofaba Desulfofrigus Desulfofustis Desulfoglaeba Desulfohalobium Desulfoluna Desulfomicrobium Desulfomonas Desulfovibrio Desulfomonile Desulfomusa Desulfofaba Desulfonatronobacter Desulfonatronospira Desulfonatronovibrio Desulfonatronum Desulfonauticus Desulfonema Desulfonispora Desulfopila Desulforegula Desulforhabdus Desulforhopalus Desulfosalsimonas Desulfosarcina Desulfosoma Desulfospira Desulfosporosinus Desulfotalea Desulfothermus Desulfotignum Desulfotomaculum Desulfovermiculus Desulfovibrio Desulfovirga Desulfovirgula Desulfurella Desulfurispira Desulfurispirillum Desulfurispora Desulfurivibrio Desulfurobacterium Desulfurococcus Desulfurolobus Acidianus Desulfuromonas Desulfuromusa Dethiobacter Dethiosulfatibacter Dethiosulfovibrio Devosia Devriesea Dialister Diaminobutyricimonas Diaphorobacter Dichelobacter Dichotomicrobium Dickeya Dictyoglomus Dietzia Dinoroseobacter Diplocalyx Diplorickettsia Dissulfuribacter Dokdonella Dokdonia Dolosigranulum Domibacillus Donghaeana Nonlabens Donghicola Dongia Dorea Draconibacterium Duganella Dyadobacter Dyella Dysgonomonas Echinicola Echinimonas Ectothiorhodosinus Ectothiorhodospira Edaphobacter Edwardsiella Effluviibacter Pontibacter Eggerthella Paraeggerthella Eggerthella Eggerthia Ehrlichia Eikenella Eilatimonas Eionea Eisenbergiella Ekhidna Elioraea Elizabethkingia Elstera Elusimicrobium Elytrosporangium Streptomyces Empedobacter Emticicia Endobacter Endozoicomonas Enhydrobacter Enhygromyxa Ensifer Enteractinococcus Enterobacter Enterococcus Enterorhabdus Enterovibrio Entomoplasma Eoetvoesia Eperythrozoon Epibacterium Epilithonimonas Eremococcus Erwinia Erysipelothrix Erythrobacter Erythromicrobium Erythromonas Blastomonas Escherichia Ethanoligenens Eubacterium Eudoraea Euzebya Euzebyella Ewingella Excellospora Actinomadura Exiguobacterium Exilispira Extensimonas Fabibacter Facklamia Faecalibacterium Faenia Saccharopolyspora Falcivibrio Falsibacillus Falsiporphyromonas Falsirhodobacter Falsochrobactrum Fangia Ferribacterium Ferrimicrobium Ferrimonas Ferriphaselus Ferrithrix Ferroglobus Ferroplasma Ferrovibrio Ferruginibacter Fervidicella Fervidicoccus Fervidicola Fervidobacterium Fibrella Fibrisoma Fibrobacter Fictibacillus Filibacter Filifactor Filimonas Filobacillus Filomicrobium Fimbriimonas Finegoldia Flagellimonas Flammeovirga Flavffiexus Flavihumibacter Flavimonas—synonym: Pseudomonas Flaviramulus Flavisolibacter Flavitalea Flavivirga Flavobacterium Flavonifractor Flectobacillus Flexibacter Flexistipes Flexithrix Flexivirga Flindersiella Fluoribacter Fluviicola Fluviimonas Fodinibacter Fodinibius Fodinicola Fodinicurvata Fontibacillus Fontibacter Fonticella Fontimonas Formivibrio Formosa Francisella Frankia Frateuria Fretibacter Fretibacterium Friedmanniella Frigoribacterium Frischella Frondicola Frondihabitans Frondihabitans Fructobacillus Fuchsiella Fulvibacter Fulvimarina Fulvimonas Fulvitalea Fulvivirga Fundibacter Alcanivorax Fusibacter Fusicatenibacter Fusobacterium Gaetbulibacter Gaetbulicola Gaetbulimicrobium Aquimarina Gaiella Galbibacter Galbitalea Galenea Gallaecimonas Gallibacterium Gallicola Gallionella Gangjinia Garciella Gardnerella Gelidibacter Gelria Gemella Geminicoccus Gemmata Gemmatimonas Gemmiger Gemmobacter Geoalkalibacter Geobacillus Geobacter Geodermatophilus Geofilum Geoglobus Geojedonia Geomicrobium Geopsychrobacter Georgenia Georgfuchsia Geosporobacter Geothermobacter Geothrix Geotoga Geovibrio Gibbsiella Giesbergeria Gilliamella Gillisia Gilvibacter Gilvimarinus Glaciecola Glaciibacter Glaciihabitans Glaciimonas Globicatella Gluconacetobacter Gluconobacter Glycocaulis Glycomyces Goodfellowiella Gordonia Gordonibacter Gracilibacillus Gracilibacter Gracilimonas Grahamella Bartonella Gramella Granulibacter Granulicatella Granulicella Granulicoccus Granulosicoccus Grimontia Gryllotalpicola Guggenheimella Gulbenkiania Gulosibacter Haematobacter Haemobartonella Haemophilus Hafnia Hahella Haladaptatus Halalkalibacillus Halalkalicoccus Halanaerobacter Halanaerobaculum Halanaerobium Halarchaeum Halarsenatibacter Haliangium Haliea Halioglobus Haliscomenobacter Hallella Haloactinobacterium Haloactinopolyspora Haloactinospora Haloarchaeobius Haloarchaeum Haloarcula Halobacillus Halobacterium Halobacteroides Halobaculum Halobellus Halobiforma Halocella Halochromatium Halococcus Haloechinothrix Haloferax Haloferula Halogeometricum Haloglycomyces Halogranum Halohasta Haloincola Halolactibacillus Halolamina Halomethanococcus Halomicroarcula Halomicrobium Halomonas Halonatronum Halonotius Halopelagius Halopenitus Halopiger Haloplanus Haloplasma Haloquadratum Halorhabdus Halorhodospira Halorientalis Halorubellus Halorubrobacterium Halorubrum Halorussus Halosarcina Halogeometricum Halosimplex Halospina Halostagnicola Halotalea Haloterrigena Halothermothrix Halothiobacillus Halovenus Halovibrio Halovivax Hamadaea Hansschlegelia Haploangium Haslfibacter Hazenella Helcobacillus Helcococcus Helicobacter Heliimonas Heliobacillus Heliobacterium Heliophilum Heliorestis Heliothrix HeIlea Henriciella Hephaestia Herbaspirillum Herbiconiux Herbidospora Herminiimonas Herpetosiphon Hespelfia Hippea Hirschia Histophilus Hoeflea Holdemania Hollandina Holophaga Holospora Homoserinimonas Hongia Hongiella Hoppeia Howardella Hoyosella Huaishuia Huanghella Humibacillus Humibacter Humicoccus Nakamurella Humihabitans Intrasporangium Humitalea Hungatella Hwangdonia Hwanghaeicola Hyalangium Hydrocarboniphaga Hydrogenibacillus Hydrogenimonas Hydrogenispora Hydrogenivirga Hydrogenoanaerobacterium Hydrogenobacter Hydrogenobaculum Hydrogenophaga Hydrogenophilus Hydrogenothermus Hydrogenovibrio Hydrotalea Hylemonella Hymenobacter Hyperthermus Hyphomicrobium Hyphomonas Hyunsoonleella lamia Ideonella Idiomarina Ignatzschineria Ignavibacterium Ignavigranum lgnicoccus Ignisphaera Ilumatobacter Ilyobacter Imperialibacter Imtechella Indibacter Inhella Inquilinus Insolitispirillum Intesfinibacter lntestinimonas Intrasporangium Iodobacter lsobaculum Isochromatium Isoptericola Isosphaera Jahnella Janibacter Jannaschia Janthinobacterium Jatrophihabitans Jejudonia Jejuia Jeongeupia Jeotgalibaca Jeotgalibacillus Jeotgalicoccus Jhaorihella Jiangella Jishengella Johnsonella Jonesia Jonquetella Joostella Kaistella Chryseobacterium Kaistia Kallotenue Kandleria Kangiella Kerstersia Ketogulonicigenium Kibdelosporangium Kiloniella Kineococcus Kineosphaera Kineosporia Kingella Kinneretia Kistimonas Kitasatoa Streptomyces Kitasatospora Klebsiella Klugiella Kluyvera Knoellia Kocuria Kofleria Komagataeibacter Kordia Kordiimonas Koreibacter—synonym: Paraoerskovia Kosakonia Koserella Kosmotoga Kozakia Krasilnikovia Kribbella Kribbia Kriegella Krokinobacter Dokdonia Kroppenstedtia Ktedonobacter [Ktedobacter] Kurthia Kushneria Kutzneria Kyrpidia Kytococcus Labedaea Labedella Labrenzia Labrys Laceyella Lachnoanaerobaculum Lachnobacterium Lachnospira Lacibacter Lucibacterium Lacinutrix Lacticigenium Lactivibrio Lactobacillus Lactococcus Lactonifactor Lactosphaera Trichococcus Lactovum Lamprobacter Lamprocystis Lampropedia Lapillicoccus Laribacter Larkinella Lautropia Lawsonia Leadbetterella Lebetimonas Lechevalieria Leclercia Leeia Leeuwenhoekiella Legionella Leifsonia Leisingera Lelliottia Leminorella Lentibacillus Lentibacter Lentilitoribacter Lentisphaera Lentzea Leptobacterium Leptolinea Leptonema Leptospira Leptospirillum Leptothrix Leptotrichia Leucobacter Leuconostoc Leucothrix Levilinea Levinea Lewinella “Liberibacter” Lihuaxuella Limibacter Limimonas Limnobacter Limnohabitans Lishizhenia Listeria Listonella Litoreibacter Litoribacillus Litoribacter Litoricola Litorilinea Litorimicrobium Litorimonas Litorisediminicola Loktanella Lonepinella Longilinea Longimycelium Longispora Lonsdalea Lucibacterium Vibrio Luedemannella Luminiphilus Lutaonella Luteibacter Luteibaculum Luteimicrobium Luteimonas Luteipulveratus Luteivirga Luteococcus Luteolibacter Lutibacter Lutibaculum Lutimaribacter Lutimonas Lutispora Lysinibacillus Lysinimicrobium Lysinimonas Lysobacter Lyticum Macellibacteroides Macrococcus Macromonas Magnetococcus Magnetospira Magnetospirillum Magnetovibrio Mahella Malikia Malonomonas Mameliella Mangrovibacter Mangrovibacterium Mangroviflexus Mangrovimonas Mannheimia Maribacter Maribaculum Henriciella Maribius Maricaulis Marichromatium Maricurvus Marihabitans Marinactinospora Marinibacillus Jeotgalibacillus Marinicauda Marinicella Marinicola Roseivirga Marinifilum Mariniflexile Marinilabilia Marinilactibacillus Mariniluteicoccus Marinimicrobium Marininema Mariniradius Marinithermus Marinitoga Marinivirga Algibacter Marinobacter Marinobacterium Marinococcus Marinomonas Marinoscillum Marinospirillum Marinovum Mariprofundus Marisediminicola Marispirillum Maritalea Maritimibacter Maritimimonas Mari virga Marivita Marixanthomonas Marmoricola Martelella Marvinbryantia Massilia Mechercharimyces Megamonas Meganema Megasphaera Meiothermus Melaminivora Melghirimyces Melioribacter Melissococcus Melitea—synonym: Spongiibacter Melittangium Meniscus Meridianimaribacter Mesoaciditoga Mesoflavibacter Mesonia Mesophilobacter Mesoplasma Mesorhizobium Mesotoga Metallibacterium Metallosphaera Metascardovia Alloscardovia Methanimicrococcus Methanobacterium Methanobrevibacter Methanocalculus Methanocaldococcus Methanocella Methanococcoides Methanococcus Methanocorpusculum Methanoculleus Methanofollis Methanogenium Methanohalobium Methanohalophilus Methanolacinia Methanolinea Methanolobus Methanomassiliicoccus Methanomethylovorans Methanomicrobium Methanoplanus Methanopyrus Methanoregula Methanosaeta Methanosalsum Methanosarcina Methanosphaera Methanospirillum Methanothermobacter Methanothermococcus Methanothermus Methanothrix Methanosaeta Methanotorris Methermicoccus Methylarcula Methylibium Methylobacillus Methylobacter Methylobacterium Methylocaldum Methylocapsa Methyloceanibacter Methylocella Methylococcus Methylocystis Methyloferula Methylogaea Methylohalobius Methylohalomonas Methyloligella Methylomarinovum Methylomarinum Methylomicrobium Methylomonas Methylonatrum Methyloparacoccus Methylophaga Methylophilus Methylopila Methylorhabdus Methylorosula Methylosarcina Methylosinus Methylosoma Methylosphaera Methylotenera Methylothermus Methyloversatilis Methylovirgula Methylovorus Methylovulum Mica vibrio Microaerobacter Microbacterium Microbispora Microbulbifer Microcella Micrococcus Microcyclus Ancylobacter Microellobosporia Microlunatus Micromonas Micromonospora Micropolyspora Micropruina Microscilla Microsphaera Nakamurella Microterricola Microtetraspora Micro virga Microvirgula Millisia Miniimonas Mitsuaria Mitsuokella Mobilicoccus Mobiluncus Modestobacter Modicisalibacter Moellerella Mogibacterium Moheibacter Mongoliicoccus Mongoliitalea Mooreia Moorella Moraxella Morganella Moritella Morococcus Moryella Motilibacter Mucilaginibacter MucispirillumMumia Murdochiella Muricauda Muricoccus Roseomonas Muriicola Murinocardiopsis Myceligenerans Mycetocola Mycobacterium Mycoplana Mycoplasma Myroides Myxococcus Naasia Nafulsella Nakamurella Namhaeicola Nannocystis Natranaerobaculum Natranaerobius Natranaerovirga Natrialba Natribacillus Natrinema Natroniella Natronincola Natronoarchaeum Natronobacillus Natronobacterium Natronocella Natronococcus Natronoflexus Natronolimnobius Natronomonas Natronorubrum Natronovirga Naumannella Nautella Nautilia Naxibacter Massilia Necropsobacter Negativicoccus Neiella Neisseria Neoasaia Neochlamydia Neokomagataea Neorickettsia Neptuniibacter Neptunomonas Nereida Nesiotobacter Nesterenkonia Nevskia Nguyenibacter Niabella Niastella Nibrella Nibribacter Nicoletella Nisaea Nitratifractor Nitratireductor Nitratiruptor Nitriliruptor Nitrincola Nitritalea Nitrobacter Nitrococcus Nitrolancea Nitrosococcus Nitrosolobus Nitrosospira Nitrosomonas Nitrosospira Nitrospina Nitrospira Nocardia Nocardioides Nocardiopsis Nonlabens Nonomuraea Noviherbaspirillum Novispirillum Novosphingobium Nubsella Obesumbacterium Oceanibacterium Oceanibaculum Oceanibulbus Oceanicaulis Oceanicella Oceanicola Oceanimonas Oceanirhabdus Oceaniserpentilla Oceanisphaera Oceanitalea Oceanithermus Oceanobacillus Oceanobacter Oceanococcus Oceanospirillum Oceanotoga Ochrobactrum Octadecabacter Odoribacter Oenococcus Oerskovia Ohtaekwangia Okibacterium Oleibacter Oleiphilus Oleispira Oligella Oligosphaera Oligotropha Olivibacter Olleya Olsenella Opitutus Orbus Orenia Oribacterium Oribaculum Porphyromonas Orientia Ornatilinea Omithinibacillus Ornithinibacter Ornithinicoccus Ornithinimicrobium Ornithobacterium Oryzihumus Oscillibacter Oscillochloris Oscillospira Otariodibacter Ottowia Owenweeksia Oxalicibacterium Oxalobacter Oxalophagus Oxobacter Pacificibacter Paenalcaligenes Paenibacillus Paenirhodobacter Paenisporosarcina Paenochrobactrum Palaeococcus Palleronia Paludibacter Paludibacterium Panacagrimonas Pandoraea Pannonibacter Pantoea Papillibacter Parabacteroides Parachlamydia Paracoccus Paracraurococcus Paraeggerthella Paraferrimonas Paraherbaspirillum Paralactobacillus Lactobacillus Paralcaligenes Paraliobacillus Paramoritella Paraoerskovia Parapedobacter Paraperlucidibaca Paraprevotella Parapusillimonas Pararhodobacter Pararhodospirillum Parascardovia Parasegetibacter Parasphingopyxis Parasporobacterium Parasutterella Parvibacter Parvibaculum Parvimonas Parvularcula Pasteurella Pasteuria Patulibacter Paucibacter Paucimonas Paucisalibacillus Pectinatus Pectobacterium Pediococcus Pedobacter Pedomicrobium Pelagibaca Pelagibacillus Terribacillus Pelagibacterium Pelagibius Pelagicoccus Pelagicola Pelagimonas Pelczaria Pelistega Pelobacter “Pelodictyon” Pelomonas Pelosinus Pelospora Pelotomaculum Peptococcus Peptoniphilus Peptostreptococcus Peredibacter Perexilibacter Perlucidibaca Persephonella Persicirhabdus Persicitalea Persicivirga Persicobacter Petrimonas Petrobacter Tepidiphilus Petrolinea Petrotoga Pfennigia Lamprocystis Phaeobacter Phaeochromatium Phaeocystidibacter Phaeospirillum Phaeovibrio Phascolarctobacterium Phaselicystis Phaseolibacter Phenylobacterium Phocaeicola Phocoenobacter Phorcysia Photobacterium Photorhabdus Phreatobacter Phycicoccus Phycicola Phycisphaera Phyllobacterium Phytohabitans Phytomonospora Pibocella Picrophilus Pigmentiphaga Pilibacter Pilimelia Pillotina Pimelobacter Pirella Pirellula Piscibacillus Piscicoccus Pisciglobus Piscinibacter Piscirickettsia Planctomyces Planifilum Planktomarina Planktotalea Planobacterium Chryseobacterium Planobispora Planococcus Planomicrobium Planomonospora Planopolyspora Catenuloplanes Planosporangium Planotetraspora Plantactinospora Plantibacter Plasticicumulans Pleionea Pleomorphobacterium Pleomorphomonas Plesiocystis Plesiomonas Pluralibacter Polaribacter Polaromonas Polyangium Polycladomyces Polymorphobacter Polymorphospora Polynucleobacter Pontibaca Pontibacillus Pontibacter Ponticaulis Ponticoccus Pontimonas Pontirhabdus Algibacter Porphyrobacter Porphyromonas Porticoccus Poseidonocella Postechiella Pragia Prauserella Prevotella Pricia Primorskyibacter Prochloron Prochlorothrix Profundibacterium Prolinoborus Prolixibacter Promicromonospora Propionibacter Propionivibrio Propionibacterium Propionicicella Propioniciclava Propionicimonas Propioniferax Propionigenium Propionimicrobium Propionispira Propionispora Propionivibrio Prosthecobacter Prosthecochloris Prosthecomicrobium Proteiniborus Proteiniclasticum Proteiniphilum Proteinivorax Proteocatella Proteus Protomonas Methylobacterium Providencia Pseudacidovorax Pseudahrensia Pseudaminobacter Pseudarcicella Pseudenhygromyxa Pseudidiomarina ldiomarina Pseudoalteromonas Pseudoamycolata Pseudonocardia Pseudobacteroides Pseudobutyrivibrio Pseudocaedibacter Pseudochrobactrum Pseudoclavibacter Pseudoduganella Pseudoflavonifractor Pseudofulvibacter Pseudofulvimonas Pseudogulbenkiania Pseudohaliea Pseudokineococcus Pseudolabrys Pseudomaricurvus Pseudomonas Pseudonocardia Pseudopedobacter Pseudoramibacter Pseudorhodobacter Pseudorhodoferax Pseudoruegeria Pseudosphingobacterium Pseudospirillum Pseudosporangium Pseudoteredinibacter Pseudothermotoga Pseudo vibrio Pseudoxanthobacter Pseudoxanthomonas Pseudozobellia Psychrilyobacter Psychrobacillus Psychrobacter Psychroflexus Psychroglaciecola Psychromonas Psychroserpens Psychrosphaera Pullulanibacillus Puniceibacterium Puniceicoccus Pusillimonas Pustulibacterium Pyramidobacter Pyrinomonas Pyrobaculum Pyrococcus Pyrodictium Pyrolobus Pyxidicoccus [Pyxicoccus] Quadricoccus Quatrionicoccus Quadrisphaera Quatrionicoccus Quinella Rahnella Ralstonia Ramlibacter Raoultella Rapidithrix Rarobacter Rathayibacter Rehaibacterium Reichenbachia Reichenbachiella Reichenbachiella Reinekea Renibacterium Reyranella Rhabdochromatium Rhabdothermus Rheinheimera Rhizobacter Rhizobium Rhizomicrobium Rhizomonas Sphingomonas Rhizorhabdus Rhizorhapis Rhodanobacter Rhodobaca Rhodobacter Rhodobium Rhodoblastus Rhodocista Rhodococcus Rhodocyclus Rhodocytophaga Rhodoferax Rhodoglobus Rhodoligotrophos Rhodomicrobium Rhodonellum Rhodopila Rhodopirellula Rhodoplanes Rhodopseudomonas Rhodospira Rhodospirillum Rhodothalassium Rhodothermus Rhodovarius Rhodovibrio Rhodovulum Rickettsia Rickettsiella Riemerella Rikenella Rivibacter Rivicola Robertkochia Robiginitalea Robiginitomaculum Robinsoniella Rochalimaea Bartonella Romboutsia Roseateles Roseburia Roseibaca Roseibacillus Roseibacterium Roseibium Roseicitreum Roseicyclus Roseiflexus Roseimicrobium Roseinatronobacter Roseisalinus Roseivirga Roseivivax Rosenbergiella Roseobacter Roseococcus Roseomonas Roseospira Roseospirillum Roseovarius Rothia Ruania Rubellimicrobium Rubribacterium Rubricoccus Rubrimonas Rubritalea Rubritepida Rubrivirga Rubrivivax Rubrobacter Rudaea Rudaeicoccus Rudaibacter Rudanella Ruegeria Rufibacter Rugamonas Rugosimonospora Ruminobacter Ruminococcus Rummellibacillus Runella Sabulilitoribacter Saccharibacillus Saccharibacter Saccharicrinis Saccharobacter Saccharococcus Saccharofermentans Saccharomonospora Saccharophagus Saccharopolyspora Saccharospirillum Saccharothrix Sagittula Salana Salegentibacter Salibacillus Virgibacillus Salicola Salimesophilobacter Salimicrobium Salinactinospora Salinarchaeum Salinarimonas Salinibacillus Salinibacter Salinibacterium Salinicoccus Salinicola Salinigranum Salinihabitans Salinimicrobium Salinimonas Salinirepens Salinirubrum Salinisphaera Salinispora Salinivibrio Salipiger Salirhabdus Salisaeta Salisediminibacterium Saliterribacillus Salmonella Salsuginibacillus Samsonia Sandaracinobacter Sandaracinus Sandarakinorhabdus Sandarakinotalea Nonlabens Sanguibacter Saprospira Sarcina Sarcobium Legionella Saxeibacter Nakamurella Scardovia Schineria lgnatzschineria Schlegelella Schleiferia Schlesneria Schumannella Schwartzia Sciscionella Sebaldella Sedimentibacter Sedimenticola Sediminibacillus Sediminibacter Sediminibacterium Sediminicola Sediminihabitans Sediminimonas Sediminitomix Segetibacter Segniliparus Seinonella Sejongia Chryseobacterium Selenihalanaerobacter Seleniivibrio Selenomonas Seliberia Senegalimassilia Seohaeicola Seonamhaeicola Serinibacter Serinicoccus Serpens Serpula Brachyspira Serpulina Brachyspira Serratia Sharpea Shewanella Shigella Shimazuella Shimia Shimwellia Shinella Shivajiella Shuttleworthia Siansivirga Silanimonas Silicibacter Ruegeria Silvimonas Simiduia Simkania Simonsiella Simplicispira Singularimonas Solimonas Singulisphaera Sinibacillus Sinobaca Sinobacter Solimonas Sinobacterium Sinococcus Sinobaca Sinomicrobium Sinomonas Sinorhizobium Sinosporangium Siphonobacter Skermanella Skermania Slackia Smaragdicoccus Smithella Sneathia Sneathiella Snodgrassella Snuella Sodalis Soehngenia Solibacillus Solimonas Solirubrobacter Solitalea Solobacterium Soonwooa Sorangium Spelaeicoccus Sphaerisporangium Sphaerobacter Sphaerochaeta Sphaerosporangium Sphaerotilus Sphingobacterium Sphingobium Sphingomicrobium Sphingomonas Sphingopyxis Sphingorhabdus Sphingosinicella Spinactinospora Spirilliplanes Spirillospora Spirillum Spirochaeta Spiroplasma Spirosoma Spongiibacter Spongiibacterium Spongiimonas Spongiispira Sporacetigenium Sporanaerobacter Sporichthya Sporobacter Sporobacterium Sporocytophaga Sporohalobacter Sporolactobacillus Sporolituus Sporomusa Sporosalibacterium Sporosarcina Sporotalea Pelosinus Sporotomaculum Stackebrandtia Stakelama Staleya Sulfitobacter Stanierella Aquimarina Staphylococcus Staphylothermus Stappia Starkeya Stella Stenothermobacter Nonlabens Stenotrophomonas Stenoxybacter Steroidobacter Sterolibacterium Stetteria Stibiobacter Stigmatella Stomatobaculum Stomatococcus Rothia Streptacidiphilus Streptoalloteichus Streptobacillus Streptococcus Streptohalobacillus Streptomonospora Streptomyces Streptosporangium Streptoverticillium Streptomyces Stygiolobus Subdoligranulum Subsaxibacter Subsaximicrobium Subtercola Succinatimonas Succiniclasticum Succinimonas Succinispira Succinivibrio Sulfitobacter Sulfobacillus Sulfolobus Sulfophobococcus Sulfuricella Sulfuricurvum Sulfurihydrogenibium Sulfurimonas Sulfurisoma Sulfurisphaera Sulfuritalea Sulfurivirga Sulfurococcus Sulfurospirillum Sulfurovum Sungkyunkwania Sunxiuqinia Sutterella Suttonella Swaminathania Swingsia Symbiobacterium Symbiotes Synergistes Syntrophaceticus Syntrophobacter Syntrophobotulus Syntrophococcus Syntrophomonas Syntrophorhabdus Syntrophospora Syntrophomonas Syntrophothermus Syntrophus Tabrizicola Tahibacter Taibaiella Tamlana Tamlicoccus Tannerella Tanticharoenia Taonella Tardiphaga Tateyamaria Tatlockia Tatumella Taylorella Tectibacter Teichococcus Roseomonas Telluria Telmatobacter Telmatocola Telmatospirillum Tenacibaculum Tenuibacillus Tepidamorphus Tepidanaerobacter Tepidibacillus Tepidibacter Tepidicella Tepidimicrobium Tepidimonas Tepidiphilus Terasakiella Teredinibacter Terrabacter Terracoccus Tenibacifius Terriglobus Terrimicrobium Terrimonas Terrisporobacter Tersicoccus Tessaracoccus Tetragenococcus Tetrasphaera Tetrathiobacter Advenella Texcoconibacillus Thalassobacillus Thalassobacter Thalassobaculum Thalassobius Thalassococcus Thalassolituus Thalassomonas Thalassospira Thalassotalea Thauera Thermacetogenium Thermaerobacter Thermanaeromonas Thermanaerovibrio Thermasporomyces Thermicanus Thermincola Thermithiobacillus Thermoactinomyces Thermoactinospora Thermoanaerobacter Thermoanaerobacterium Thermoanaerobaculum Thermoanaerobium Thermobacillus Thermobacteroides Thermobifida Thermobispora Thermobrachium Thermocatellispora Thermochromatium Thermocladium Thermococcoides Kosmotoga Thermococcus Thermocrinis Thermocrispum Thermodesulfatator Thermodesulfobacterium Thermodesulfobium Thermodesulforhabdus Thermodesulfovibrio Thermodiscus Thermofilum Thermoflavifilum Thermoflavimicrobium Thermoflexus Thermogemmatispora Thermogymnomonas Thermohalobacter Thermohydrogenium Thermoleophilum Thermolithobacter Thermolongibacillus Thermomarinilinea Thermomicrobium Thermomonas Thermomonospora Thermonema Thermophagus Thermoplasma Thermopolyspora Thermoproteus Thermosediminibacter Thermosinus Thermosipho Thermosphaera Thermosporothrix Thermosuffidibacter Thermosulfurimonas Thermosyntropha Thermotalea Thermoterrabacterium Carboxydothermus Thermothrix Thermotoga Thermotunica Thermovenabulum Thermovibrio Thermovirga Thermovorax Thermovum Thermus Thioalbus Thioalkalibacter Thioalkalicoccus Thioalkalimicrobium Thioalkalispira Thioalkalivibrio Thiobaca Thiobacillus Thiobacter Thiobacterium Thiocapsa Thioclava Thiococcus Thiocystis Thiodictyon Thiofaba Thioflavicoccus Thiohalobacter Thiohalocapsa Thiohalomonas Thiohalophilus Thiohalorhabdus Thiohalospira Thiolamprovum Thiomargarita Thiomicrospira Thiomonas Thiopedia Thiophaeococcus Thioploca Thioprofundum Thioreductor Thiorhodococcus Thiorhodospira Thiorhodovibrio Thiosphaera Paracoccus Thiospira Thiospirillum Thiothrix Thiovirga Thiovulum Thorsellia Tindallia Tissierella Tistlia Tistrella Tolumonas Tomitella Tonsilliphilus Toxothrix Trabulsiella Tranquillimonas Treponema Trichlorobacter Geobacter Trichococcus Tropheryma Tropicibacter Tropicimonas Truepera Trueperella Tsukamurella Tuberibacillus Tumebacillus Turicella Turicibacter Turneriella Uliginosibacterium Ulvibacter Umboniibacter Umezawaea Undibacterium Ureaplasma Ureibacillus Uruburuella Vadicella Vagococcus Vallitalea Vampirovibrio Varibaculum Variovorax Vasilyevaea Veillonella Venenivibrio Verminephrobacter Verrucomicrobium Verrucosispora Vibrio Vibrionimonas Victivallis Virgibacillus Virgisporangium Viridibacillus Vitellibacter Vitreoscilla Vogesella Volcaniella Halomonas Volucribacter Vulcanibacillus Vulcaniibacterium Vulcanisaeta Vulcanithermus Waddlia Wandonia Wangia Zunongwangia Wautersia Cupriavidus Wautersiella Weeksella Weissella Wenxinia Wenyingzhuangia Wigglesworthia Williamsia Winogradskyella Wohlfahrtiimonas Wolbachia Wolinella Woodsholea Xanthobacter Xanthomonas Xenophilus Xenorhabdus Xiangella Xylanibacter Prevotella Xylanibacterium Xylanimicrobium Xylanimonas Xylella Xylophilus Yangia Yania Yaniella Yaniella Yeosuana Yersinia Yimella Yokenella—synonym: Koserella Yonghaparkia Youngiibacter Yuhushiella Zavarzinella Zavarzinia Zeaxanthinibacter Zhangella Maritalea Zhihengliuella Zhongshania Zhouia Zimmermannella=Pseudoclavibacter Zobellella Zobellia Zoogloea Zooshikella Zunongwangia Zymobacter Zymomonas Zymophilus.

Even more preferably, when the present invention relates to the modified microorganism, composition, vaccine, adjuvant, method or use of the present invention, said microorganism is a bacterium selected from the group consisting of Lactobacillus paracasei, Lactobacillus reuteri, Lactobacillus rahmnosus, Lactobacillus intestinalis, Lactobacillus Acidophilus, Lactobacillus murinus, Lactobacillus brevis, Bifidobacterium infantis, Bifidobacterium animalis, Bifidobacterium breves, Bifido acidifacien, Bacteroides vulgatus, Bacteroides xylanisolvens, Clostridium xynalitycum, Clostridium scidens, Eubacterium cylindroides, Enteroccocus faecalis, Enteroccocus feacium, and E. coli. Most preferably, when the present invention relates to the modified microorganism, composition, vaccine, adjuvant, method or use of the present invention, said microorganism is Lactobacillus paracasei or Lactobacillus reuteri.

In some aspects, the present invention relates to the modified microorganism, composition, vaccine, adjuvant, method or use of the present invention, wherein said microorganism is non-pathogenic, preferably said non-pathogenic is not associated with a mammalian pathological or infectious condition or disease, further preferably said mammalian pathological or infectious condition or disease is a human pathological or infectious condition or disease.

In some aspects, the present invention relates to the modified microorganism, composition, vaccine, adjuvant, method or use of the present invention, wherein said microorganism is non-pathogenic and/or opportunistic pathogen and is selected from the group consisting of genera as defined in [152 and 153] herein.

In some aspects, the present invention relates to the modified microorganism, composition, vaccine, adjuvant, method or use of the present invention, wherein said microorganism is gram-positive, non-pathogenic and/or opportunistic pathogen and is selected from the group consisting of genera as defined in [152 and 153].

In some aspects, the present invention relates to the modified microorganism, composition, vaccine, adjuvant, method or use of the present invention, wherein said microorganism is non-toxic to a mammalian host, preferably said microorganism is non-toxic to a human host.

In some aspects, the present invention relates to the modified microorganism, composition, vaccine, adjuvant, method or use of the present invention, wherein said microorganism is capable of one or more of the following characteristics: binding and/or reducing toxicity and/or neutralizing a toxin; binding and/or reducing the pathogenicity and/or neutralizing a pathogenic microorganism; binding a receptor of a toxin; binding a receptor of a pathogenic microorganism.

In some aspects, the present invention relates to the modified microorganism, composition, vaccine, adjuvant, method or use of the present invention, wherein said microorganism is isolated and/or live and/or killed and/or attenuated and/or pasteurized and/or lyophilized and/or freeze dried.

### EXAMPLES OF THE INVENTION

In order that the invention may be readily understood and put into practical effect, some aspects of the invention are described by way of the following non-limiting examples.

### Example 1: Loading Protocol

1.1. Material Methods:

Loading Protocol.

Bacterial cultures were grown in suitable media (e.g., Table 1), washed and re-suspended in PBS or DPBS at OD1 to OD10 (e.g., 600 nm) as mentioned above. Alternatively, bacteria were grown in suboptimal media (e.g., medium that does not allow optimal growth, growth rate, generation time) or a medium containing propionic acid (e.g., at concentration of 5 ml/L). Cells were than harvested through centrifugation and resuspended in PBS or DPBS. For loading the bacterial suspensions were centrifuged again and resuspended in their growth medium or a suboptimal media with 0.5 to 10 μg/ml Glycosphingolipid (e.g. GM1 or Gb3), optionally added with 1% cholesterol and incubated at 20° C. to 60° C. overnight or longer under agitation or not (e.g., Table 1). The cells were then extensively washed with PBS or DPBS or PBS 0.02% Tween 20 before being resuspended in PBS or DPBS and stored at 4° C.

Procedure for testing the presence of the lipid carrier (i.e. GM1 or Gb3) on the surface of the loaded microorganism.

Monosialoganglioside GM1, Gd1a, Gd1b, Asialo GM1, GM2 are glycosphingolipids composed of a ceramide (sphingosine and fatty Acid) and an oligosaccharide. Each lipid-carrier presents an unique carbohydrate structure. Monosialotetrahexosylganglioside red (GM1-red; reductive Ozonized GM1) is a derivative of the lipid carrier GM1 that is deprived from a sphingosine moiety. The oligosaccharide part of all lipid carriers mentioned above is known to bind the cholera toxin (CT).

Globotriaosylceramide (Gb3) is a glycosphingolipid composed of a ceramide (Sphingosine and Fatty Acid) and an oligosaccharide. N-Hexadecanyl-Ceramide-trihexoide and N-Octadecanyl-Ceramide-trihexoide are derivatives of the lipid carrier Gb3 that differ in the length of their fatty acid. N-(1-Adamantaneacetyl)-ceramide trihexoside is a derivative of the lipid carrier Gb3 for which one sphingosine was replaced with an adamantaneacetyl.

The oligosaccharide part of Gb3 is known to be a natural receptor for the shiga toxins (Stxs). Both toxins (CT and Stx) were labelled and used to identify the presence of the lipid-carrier on the surface of the loaded microorganisms.

Labeling of Toxin.

Horseradish peroxidase (HRP) labelling: Horseradish peroxidase (HRP) labelling were performed according to the manufacturer instructions using EZ-Link™ Plus Activated Peroxidase Kit (Thermo Fischer Scientific, Braunschweig, Germany, Catalog number in Mai 2019: 31489) Plus Activated Peroxidase Kit (Thermo Fisher Scientific Inc., Rockford, USA). Alkaline Phosphatase (AP) labelling: AP labelling were performed according to the manufacturer instructions using Lightning-Link® Alkaline Phosphatase Antibody Labeling Kit (Novus Biologicals Europe/UK, Abingdon, United Kingdom Catalog Number in May 2019: 702-0030).

ELISA.

Cells loaded with lipid carrier that are binding cholera toxin or shiga toxin (e.g. GM1 or Gb3) as mentioned above were diluted in PBS or DPBS with 2% HSA or BSA, respectively at OD1 to OD5 (e.g., 600 nm) and incubated at Room temperature (RT) or 37° C. for 1 hour to prevent unspecific binding. The cells were washed once, re-suspended in PBS or DPBS with 1% HSA/BSA, added with the labelled (e.g., HRP or AP) CT or STx and incubated for 1 to 2 hours at RT or 37° C.

Cells were washed 3 times with PBS or DPBS or PBS+0.02% Tween 20 and resuspended in PBS or DPBS. 50 μl of bacterial suspensions were given in triplicate on an ELISA plate. Two wells were inoculated with a suitable substrate (for HRP or AP), the reactions were stopped, and extinction measured at a suitable wavelength (e.g., 580 nm for AP, 450 and 630 (as reference) for HRP). The third well was used to determine the OD in the final cell suspension and the extinction measured previously and standardized to OD1. Standardized results allowed comparing the binding strength of different strains and/or preparations.

1.2. Results:

Loading protocol.

Cells (e.g., bacterial cells) were cultivated in suitable media under suitable conditions (e.g., Table 1). Cells were than harvested through centrifugation and resuspended at a suitable loading concentration, in suitable loading medium, at suitable loading pH, added with 0.5 to μg/ml lipid carrier (e.g. GM1, Gb3) and incubated at suitable loading temperature overnight or longer (e.g., Table 1 and procedures described above).

Surprisingly, for some bacteria (i.e. Lactobacillus, Bacteroides, Clostridium), a loading was barely possible when their optimal growth medium was used as loading medium (i.e. MRS medium for Lactobacillus strains). The best loading medium was found to be a medium in which cells were slowly or not growing (e.g., Lactobacillus seems to be better loadable in PBS-BSM, BSM or ABM, media in which they are barely growing, e.g., Table 1).

Surprisingly, with appropriate conditions (e.g., Table 1 and procedures described above), any tested cells being gram negative, gram positive or yeast could be loaded with any of the tested lipid carrier, including Lipid carrier that are deprived from sphingosine like N-1 Adamantaneacetyl-ceramide trihexoside and GM1-red.

For the purpose of optimal loading, the best cultivation temperature seems not always to be the optimal growth temperature. Temperatures used were between 30 and 55° C. (e.g., Table 1). Suitable loading concentration (of the bacteria) was found to be between OD 1 and OD 10. Suitable loading pH was found to be in the range of pH from 3 to 7 with pH from 6 to 5 being optimal (e.g., Table 1). Suitable loading media was similarly identified (e.g., Table 1). Suitable loading temperature was also identified (e.g., Table 1). Temperature used was between 30 and 55° C. (e.g., Table 1).

For the purpose of optimal loading:

TABLE 1
Correlation of the suitable combinations of specific microorganism, inoculation medium, culture medium (i.e., growth medium),
growth temperature, temperature of adding of said solubilized heterologous lipid carrier (i.e., loading temperature), pH and loading
medium with the read-outs of the Toxin Binding (e.g. CT or Stx for GM1 and Gb3 respectivelly) assays as described above.

| | | | | | | | | Cholera |
|---|---|---|---|---|---|---|---|---|
| | Inoculation | Growth | Growth | Growth | Loading | Loading | Loading | Toxin |
| Strain | Medium | Temp | Medium | Temp | Medium | Temp. | pH | Binding |
| Lactobacillus paracasei L3 & | MRS | 30° C. | | | PBS | 30° C. | 7.4 | (+/−) |
| Lactobacillus reuteri K9 | | | | | | | | |
| Lactobacillus paracasei L3 & | MRS | 37° C. | | | PBS | 30° C. | 7.4 | (+/−) |
| Lactobacillus reuteri K9 | | | | | | | | |
| Lactobacillus paracasei L3 & | MRS | 37° C. | | | PBS | 4° C. | 7.4 | (+) |
| Lactobacillus reuteri K9 | | | | | | | | |
| Lactobacillus paracasei L3 & | BSM or | 37° C. | | | BSM or | 30° C. | 6.8 or | (+) |
| Lactobacillus reuteri K9 | ABM | | | | ABM | | 5.5 | |
| Lactobacillus paracasei L3 & | BSM or | 37° C. | | | BSM or | 37° C. | 6.8 or | (+) |
| Lactobacillus reuteri K9 | ABM | | | | ABM | | 5.5 | |
| Lactobacillus paracasei L3 & | MRS | 37° C. | MRS | 37° C. | BSM or | 37° C. | 6.8 or | (++) |
| Lactobacillus reuteri K9 | | | | | ABM | | 5.5 | |
| Lactobacillus paracasei L3 & | MRS | 37° C. | MRS | 37° C. | BSM or | 30° C. | 6.8 or | (++) |
| Lactobacillus reuteri K9 | | | | | ABM | | 5.5 | |
| Lactobacillus paracasei L3 & | MRS | | BSM OR | 37° C. | PBS | 55° C. | 3 | (+) |
| Lactobacillus reuteri K9 | | | ABM | | | | | |
| Lactobacillus paracasei L3 & | MRS | 37° C. | | | BSM/PBS | 46° C. | 6 | (+) |
| Lactobacillus reuteri K9 | | | | | | | | |
| Lactobacillus paracasei L3 & | MRS | 37° C. | MRS + | 37° C. | BSM/PBS | 46° C. | 6 | (++) |
| Lactobacillus reuteri K9 | | | propionic | | | | | |
| | | | acid 5 ml/L | | | | | |
| Lactobacillus paracasei L3 & | MRS | 37° C. | | | BSM/PBS | 37° C. | 6. 6.8 | (++) |
| Lactobacillus reuteri K9 | | | | | BSM OR | | or 5.5 | |
| | | | | | ABM | | | |
| Lactobacillus paracasei L3 & | MRS | 37° C. | BSM + | 37° C. | BSM/PBS | 46° C. | 6 | (++) |
| Lactobacillus reuteri K9 | | | propionic | | | | | |
| | | | acid 5 ml/L | | | | | |
| Lactobacillus paracasei L3 & | MRS | 37° C. | BSM OR | 37° C. | BSM/PBS | 46° C. | 6 | (++) |
| Lactobacillus reuteri K9 | | | ABM | | | | | |
| Lactobacillus paracasei L3 & | MRS | 37° C. | BSM OR | 37° C. | BSM/PBS | 46° C. | 5 | (+) |
| Lactobacillus reuteri K9 | | | ABM | | | | | |
| Lactobacillus paracasei L3 & | MRS | 37° C. | BSM OR | 37° C. | BSM/PBS | 46° C. | 4 | (+) |
| Lactobacillus reuteri K9 | | | ABM | | | | | |
| Lactobacillus paracasei L3 & | MRS | 37° C. | BSM OR | 37° C. | BSM/PBS, | 37° C. | 6. 6.8 | (++) |
| Lactobacillus reuteri K9 | | | ABM | | BSM OR | | or 5.5 | |
| | | | | | ABM | | | |
| Lactobacillus paracasei L3 & | MRS | 37° C. | BSM OR | 46° C. | BSM/PBS | 37° C. | 6 | (++) |
| Lactobacillus reuteri K9 | | | ABM | | | | | |
| Lactobacillus paracasei L3 & | MRS | 37° C. | BSM OR | 46° C. | BSM/PBS | 46° C. | 6 | (++) |
| Lactobacillus reuteri K9 | | | ABM | | | | | |
| Lactobacillus paracasei L3 & | MRS | 37° C. | BSM OR | 46° C. | BSM/PBS | 55° C. | 6 | (++) |
| Lactobacillus reuteri K9 | | | ABM | | | | | |
| Lactobacillus paracasei L3 & | MRS | 37° C. | BSM OR | 55° C. | BSM/PBS | 37° C. | 6 | (++) |
| Lactobacillus reuteri K9 | | | ABM | | | | | |
| Lactobacillus paracasei L3 & | MRS | 37° C. | BSM OR | 55° C. | BSM/PBS | 46° C. | 6 | (++) |
| Lactobacillus reuteri K9 | | | ABM | | | | | |
| Lactobacillus paracasei L3 & | MRS | 37° C. | BSM OR | 55° C. | BSM/PBS | 55° C. | 6 | (++) |
| Lactobacillus reuteri K9 | | | ABM | | | | | |
| Lactobacillus paracasei L3 & | MRS | 37° C. | BSM OR | 37° C. | BSM/PBS | 37° C. | 6 | (++) |
| Lactobacillus reuteri K9 | | | ABM | | | | | |
| Lactobacillus paracasei L3 & | MRS | 37° C. | BSM OR | 37° C. | BSM/PBS | 46° C. | 6 | (++) |
| Lactobacillus reuteri K9 | | | ABM | | | | | |
| Lactobacillus paracasei L3 & | MRS | 37° C. | BSM OR | 37° C. | BSM/PBS | 55° C. | 6 | (++) |
| Lactobacillus reuteri K9 | | | ABM | | | | | |
| Lactobacillus paracasei DSM 46331) | MRS | 37° C. | BSM OR | 37° C. | ABM | 37° C. | 5.5 | (++) |
| | | | ABM | | | | | |
| Lactobacillus paracasei DSM 46331) | MRS | 37° C. | MRS | 37° C. | ABM | 37° C. | 5.5 | (++) |
| Lactobacillus Rahmnosuss (P15, P16) | MRS | 37° C. | BSM OR | 37° C. | ABM | 37° C. | 5.5 | (++) |
| | | | ABM | | | | | |
| Lactobacillus Rahmnosuss (P15, P16) | MRS | 37° C. | MRS | 37° C. | ABM | 37° C. | 5.5 | (++) |
| Lactobacillus Delbrueckii (DSM 20074) | MRS | 37° C. | BSM OR | 37° C. | ABM | 37° C. | 5.5 | (++) |
| | | | ABM | | | | | |
| Lactobacillus Delbrueckii (DSM 20074) | MRS | 37° C. | MRS | 37° C. | ABM | 37° C. | 5.5 | (++) |
| Lactobacillus acidophilus (DSM 20079) | MRS | 37° C. | BSM OR | 37° C. | ABM | 37° C. | 5.5 | (++) |
| | | | ABM | | | | | |
| Lactobacillus acidophilus (DSM 20079) | MRS | 37° C. | MRS | 37° C. | ABM | 37° C. | 5.5 | (++) |
| Lactobacillus acidophilus without LTA | MRS | 37° C. | BSM OR | 37° C. | ABM | 37° C. | 5.5 | (++) |
| | | | ABM | | | | | |
| Lactobacillus acidophilus without LTA | MRS | 37° C. | MRS | 37° C. | ABM | 37° C. | 5.5 | (++) |
| Lactobacillus intestinalis (DSM 6629) | MRS | 37° C. | MRS | 37° C. | ABM | 37° C. | 5.5 | (+) |
| Lactobacillus plantarum (DSM 20174) | MRS | 37° C. | MRS | 37° C. | ABM | 37° C. | 5.5 | (+) |
| Lactobacillus Murinus (DSM 20452) | MRS | 37° C. | MRS | 37° C. | IABM | 37° C. | 5.5 | (++) |
| Lactobacillus Brevis (DSMZ 2647) | MRS | 37° C. | MRS | 37° C. | ABM | 37° C. | 5.5 | (++) |
| Lactobacillus rahmnosus GG | MRS | 37° C. | MRS | 37° C. | ABM | 37° C. | 5.5 | (+) |
| (ATCC 53103) | | | | | | | | |
| Bifidobacterium breves (DSM 20213) | BSM OR | | BSM OR | 37° C. | ABM/PBS | 37° C. | 7.4 | (++) |
| | ABM | | ABM | | | | | |
| Bifidobacterium breves (DSM 20213) | BSM OR | | BSM OR | 37° C. | BSM/PBS | 46° C. | 6 | (++) |
| | ABM | | ABM | | | | | |
| Bifidobacterium longum (DSM 20088) | BSM OR | | BSM OR | 37° C. | ABM | 37° C. | 7.4 | (+) |
| | ABM | | ABM | | | | | |
| Bifidobacterium longum (DSM 20088) | BSM OR | | BSM OR | 37° C. | BSM/PBS | 46° C. | 6 | (++) |
| | ABM | | ABM | | | | | |
| Bifidobacterium Animalis (BA4) | BSM OR | | BSM OR | 37° C. | ABM | 3° C. | 5.5 | (++) |
| | ABM | | ABM | | | | | |
| Bifidobacterium Animalis (BA4) | MRS | | BSM OR | 37° C. | ABM | 37° C. | 5.5 | (++) |
| | | | ABM | | | | | |
| Bifidobacterium Animalis (DN 173010) | BSM OR | | BSM OR | 37° C. | ABM | 37° C. | 5.5 | (++) |
| | ABM | | ABM | | | | | |
| Bifidobacterium. Acidifaciens | BSM OR | | BSM OR | 37° C. | ABM | 37° C. | 5.5 | (++) |
| (DSM1 5896) | ABM | | ABM | | | | | |
| Bifidobacterium infantis (longum) | BSM OR | | BSM OR | 37° C. | ABM | 37° C. | 5.5 | (++) |
| (ATCC 15697) | ABM | | ABM | | | | | |
| Bacteroides vulgatus BV5 | WC | | WC | 37° C. | PBS/ABM | 37° C. | 7.4 | (+) |
| Bacteroides vulgatus BV5 | BSM OR | | BSM OR | 37° C. | PBS | 37° C. | 7.4 | (++) |
| | ABM | | ABM | | | | | |
| Bacteroides vulgatus BV5 | WC | | WC | 37° C. | BSM/PBS | 46° C. | 6 | (+) |
| Bacteroides vulgatus BV5 | BSM or | | BSM or | 37° C. | BSM/PBS | 46° C. | 6 | (++) |
| | ABM | | ABM | | | | | |
| Bacteroides vulgatus (DSM 1447) | WC | | WC | 37° C. | BSM/PBS | 37° C. | 6 | (+) |
| Bacteroides vulgatus (DSM 1447) | BSM or | | BSM or | 37° C. | BSM/PBS | 37° C. | 6 | (++) |
| | ABM | | ABM | | | | | |
| Enterococcus faecalis S12 | MRS | | MRS | 37° C. | BSM/PBS | 46° C. | 6 | (+) |
| Enterococcus faecalis S12 | MRS + | | MRS + | 37° C. | BSM/PBS | 46° C. | 6 | (++) |
| | propionic | | propionic | | | | | |
| | acid 5 ml/L | | acid 5ml/L | | | | | |
| E. Coli (DSM 613) | LB | | BSM or | 37° C. | BSM/PBS | 37° C. | 6 | (++) |
| | | | ABM | | | | | |
| E. Coli (DSM 6601) | LB | | BSM or | 37° C. | BSM/PBS | 37° C. | 6 | (++) |
| | | | JABM | | | | | |
| Eubact. cylindroides (ATCC 27803) | TSB | | BSM or | 37° C. | BSM/PBS | 37° C. | 6 | (++) |
| | | | ABM | | | | | |
| Clostridium xylanilyticum (DSM 6555) | BHI | | BSM or | 37° C. | BSM/PBS | 37° C. | 6 | (+) |
| | | | ABM | | | | | |
| Clostridium xylanilyticum (DSM 6555) | BHI | | BSM or | 37° C. | BSM/PBS | 46° C. | 6 | (+) |
| | | | ABM | | | | | |
| Clostridium scindens (DSM 5676) | BHI | | BSM or | 37° C. | BSM/PBS | 37° C. | 6 | (++) |
| | | | ABM | | | | | |
| saccharomyces cerevisiae | YEPD | 37° C. | | | BSM/PBS | 37° C. | 6 | (+) |
| saccharomyces boulardii | YEPD | 37° C. | | | BSM/PBS | 37° C. | 6 | (+) |

Lipid carrier: Gd1a

| | | | | | | | | |
|---|---|---|---|---|---|---|---|---|
| Lactobacillus paracasei L3 & | MRS | 37° C. | BSM or | 37° C. | BSM/PBS | 37° C. | 6 | (+) |
| Lactobacillus reuteri K9 | | | ABM | | | | | |

Lipid carrier: Gd1b

| | | | | | | | | |
|---|---|---|---|---|---|---|---|---|
| Lactobacillus paracasei L3 & | MRS | 37° C. | BSM or | 37° C. | BSM/PBS | 37° C. | 6 | (+) |
| Lactobacillus reuteri K9 | | | ABM | | | | | |

Lipid carrier: Asialo GM1

| | | | | | | | | |
|---|---|---|---|---|---|---|---|---|
| Lactobacillus paracasei L3 & | MRS | 37° C. | BSM or | 37° C. | BSM/PBS | 37° C. | 6 | (+) |
| Lactobacillus reuteri K9 | | | ABM | | | | | |

Lipid carrier: GM1-Red

| | | | | | | | | |
|---|---|---|---|---|---|---|---|---|
| Lactobacillus paracasei L3 & | MRS | 37° C. | BSM or | 37° C. | BSM/PBS | 37° C. | 6 | (++) |
| Lactobacillus reuteri K9 | | | ABM | | | | | |
| Lactobacillus paracasei DSM 46331 | MRS | 37° C. | ABM | 37° C. | BSM or | 37° C. | 6.8 | (++) |
| | | | | | ABM | | | |

Lipid carrier: GM2

| | | | | | | | | |
|---|---|---|---|---|---|---|---|---|
| Lactobacillus paracasei L3 & | MRS | 37° C. | BSM or | 37° C. | BSM/PBS | 37° C. | 6 | (+) |
| Lactobacillus reuteri K9 | | | ABM | | | | | |
| Lactobacillus Rahmnosuss P15, P16 | MRS | 37° C. | ABM | 37° C. | BSM | 3° C. | 6.8 | (+) |

Lipid carrier: Gb3

| | | | | | | | |
|---|---|---|---|---|---|---|---|
| Lactobacillus paracasei L3 & | MRS | BSM or | 37° C. | BSM/PBS | 37° C. | 6 or 5.5 | (+) |
| Lactobacillus reuteri K9 | | ABM | | or ABM | | | |
| Lactobacillus paracasei L3 & | MRS | | | BSM/PBS | 37° C. | 6 or 5.5 | (+) |
| Lactobacillus reuteri K9 | | | | or ABM | | | |
| Lactobacillus paracasei L3 & | MRS | | | Modified | 37° C. | 5.5 | (+) |
| Lactobacillus reuteri K9 | | | | ABM Nr. 1 | | | |
| Lactobacillus paracasei L3 & | MRS | | | Modified | 37° C. | 5.5 | (+) |
| Lactobacillus reuteri K9 | | | | ABM Nr. 2 | | | |
| Lactobacillus paracasei L3 & | MRS | | | Modified | 37° C. | 5.5 | (+) |
| Lactobacillus reuteri K9 | | | | ABM Nr. 3 | | | |
| Lactobacillus acidophilus (DSM 20079) | MRS | BSM or | 37° C. | BSM/PBS | 37° C. | 6 or 5.5 | (+) |
| | | ABM | | or ABM | | | |
| Lactobacillus acidophilus (DSM 20079) | MRS | | | BSM/PBS | 37° C. | 6 or 5.5 | (+) |
| | | | | or ABM | | | |
| Lactobacillus Delbrueckii (DSM 20074) | MRS | BSM or | 37° C. | BSM/PBS | 37° C. | 6 or 5.5 | (+) |
| | | ABM | | or ABM | | | |
| Lactobacillus Delbrueckii (DSM 20074) | MRS | | | BSM/PBS | 37° C. | 6 or 5.5 | (+) |
| | | | | or ABM | | | |
| Bifidobacterium Animalis (BA4) | BSM | BSM or | 37° C. | BSM/PBS | 37° C. | 6 | (+) |
| | | ABM | | | | | |
| Bifidobacterium Animalis (BA4) | MRS | | | BSM/PBS | 37° C. | 6 | (+) |
| Bacteroides vulgatus (DSM 1447) | WC | BSM or | 37° C. | BSM/PBS | 37° C. | 6 or 5.5 | (+) |
| | | ABM | | or ABM | | | |
| Bacteroides vulgatus (DSM 1447) | WC | | | BSM/PBS | 37° C. | 6 or 5.5 | (+) |
| | | | | or ABM | | | |
| Enterococcus faecalis S12 | MRS | BSM or | 37° C. | ABM | 37° C. | 5.5 | (+) |
| | | ABM | | | | | |

Lipid carrier: N-Hexadecanoyl-Ceramide-trihexoide

| | | | | | | | | |
|---|---|---|---|---|---|---|---|---|
| Lactobacillus paracasei L3 & | MRS | 37° C. | BSM or | 37° C. | BSM/PBS | 37° C. | 6 or 5.5 | (+) |
| Lactobacillus reuteri K9 | | | ABM | | or ABM | | | |
| Lactobacillus paracasei L3 & | MRS | 37° C. | BSM or | 37° C. | BSM/PBS | 46° C. | 6 or 5.5 | (+) |
| Lactobacillus reuteri K9 | | | ABM | | or ABM | | | |
| Bacteroides vulgatus (DSM 1447) | WC | | BSM or | 37° C. | BSM/PBS | 37° C. | 6 or 5.5 | (+) |
| | | | ABM | | or ABM | | | |
| Bacteroides vulgatus (DSM 1447) | WC | | BSM or | 37° C. | BSM/PBS | 46° C. | 6 or 5.5 | (+) |
| | | | ABM | | or ABM | | | |

Lipid carrier: N-Octadecanoyl-Ceramide-trihexoide

| | | | | | | | | |
|---|---|---|---|---|---|---|---|---|
| Lactobacillus paracasei L3 & | MRS | 37° C. | BSM or | 37° C. | BSM/PBS | 37° C. | 6 or 5.5 | (+) |
| Lactobacillus reuteri K9 | | | ABM | | or ABM | | | |
| Lactobacillus paracasei L3 & | MRS | 37° C. | BSM or | 37° C. | BSM/PBS | 46° C. | 6 or 5.5 | (+) |
| Lactobacillus reuteri K9 | | | ABM | | or ABM | | | |
| Bacteroides vulgatus (DSM 1447) | WC | | BSM or | 37° C. | BSM/PBS | 37° C. | 6 or 5.5 | (+) |
| | | | ABM | | or ABM | | | |
| Bacteroides vulgatus (DSM 1447) | WC | | BSM or | 37° C. | BSM/PBS | 46° C. | 6 or 5.5 | (+) |
| | | | ABM | | or ABM | | | |

Lipid carrier: N-(1-Adamantaneacetyl)-ceramide trihexoside (N-(1-Adamantaneacetyl)-Gb3)

| | | | | | | | | |
|---|---|---|---|---|---|---|---|---|
| Lactobacillus paracasei L3 & | MRS | 37° C. | BSM or | 37° C. | BSM/PBS | 37° C. | 6 or 5.5 | (+) |
| Lactobacillus reuteri K9 | | | ABM | | or ABM | | | |

MRS: De Man, Rogosa and Sharpe Medium. BSM: Bifidobacterium Selective Medium. ABM: ACARYON Bifidobacterium Medium. “(+/−)”— weak binding, “(+)”— average binding, “(++)”— strong binding. WC Broth: Tryptone 10 g/L; Gelatin peptone 10 g/L; Yeast extract 5 g/L; Glucose 1 g/L; Sodium chloride 5 g/L; L-Arginine 1 g/L; Sodium pyruvate 1 g/L; Menadione 0.0005 g/L; Haemin 0.005 g/L (pH=7.1±0.2). WC Agar: Tryptone 10 g/L; Gelatin peptone 10 g/L; Yeast extract 5 g/L; Glucose 1 g/L; Sodium chloride 5 g/L; L-Arginine 1 g/L; Sodium pyruvate 1 g/L; Menadione 0.0005 g/L; Haemin 0.005 g/L; Agar 10 g/L (pH=7.1±0.2). MRS Broth: Peptone 10 g/l; Yeast extract 4 g/l; Beef extract 8 g/l; Glucose 20 g/l; Dipotassium phosphate 2 g/l; Sodium acetate 5 g/l; Ammonium citrate 2 g/l; Magnesium sulphate (MgSO₄) 0.2 g/l; Manganese sulphate 0.05 g/l; Polysorbate 80 1 g/l; pH=6.2±0.2. MRS Agar: Peptone 10 g/l; Yeast extract 4 g/l; Beef extract 8 g/l; Glucose 20 g/l; Dipotassium phosphate 2 g/l; Sodium acetate 5 g/l; Ammonium citrate 2 g/l; Magnesium sulphate (MgSO₄) 0.2 g/l; Manganese sulphate 0.05 g/l; Polysorbate 80 1 g/l; Agar 10 g/l; pH=6.2±0.2. BSM Agar: (Sigma Aldrich Catalog Number in May 2019: 88517). Principle and Interpretation: BSM contains Peptone and Meat extract as sources of carbon, nitrogen, vitamins and minerals. Yeast extract supplies B-complex vitamins which stimulate bacterial growth. Dextrose is the carbohydrate source. Sodium chloride maintains the osmotic balance. There is a compound in low concentration for detoxify metabolic by-products. The medium contains reducing and buffering agents. Selective salts inhibit the growth of molds, Enterococci and other Gram-negative bacteria. Another compound inhibits glycolysis by inactivating glyceraldehyde-3-phosphate dehydrogenase present and important in different bacteria and fungi (also Streptococci sp.). Three antibiotics are the selective agents and inhibit the accompanying bacterial flora like Bacilli, Enterobacteriaceae and Pseudomonas. Bifidobacteria that can reduce an azo compound present in the medium, which gives the colonies a pink-purple coloration. ABM agar contains casein peptone (12 g/l), meat peptone (5 g/l), sodium chloride (5 g/l), beef extract (3 g/l), yeast extract (3 g/l), cornstarch (1 g/l), glucose (2.5 g/l), lactulose (2.5 g/l), cysteine-hydrochloride (0.5 g/l), riboflavin (0.01 g/l), propionic acid (99%; 5 ml/1) and Agar 10 g/l. All components were mixed and suspended in distilled water. The pH was adjusted to 5.5+/−0.2 with 5N NaOH.

BSM Broth: (Sigma Aldrich Catalog Number in May 2019: 90273). Principle and Interpretation: BSM contains Peptone and Meat extract as sources of carbon, nitrogen, vitamins and minerals. Yeast extract supplies B-complex vitamins which stimulate bacterial growth. Dextrose is the carbohydrate source. Sodium chloride maintains the osmotic balance. There is a compound in low concentration for detoxify metabolic by-products. The medium contains reducing and buffering agents. Selective salts inhibit the growth of molds, Enterococci and other Gram-negative bacteria. Another compound inhibits glycolysis by inactivating glyceraldehyde-3-phosphate dehydrogenase present and important in different bacteria and fungi (also Streptococci sp.). Three antibiotics are the selective agents and inhibit the accompanying bacterial flora like Bacilli, Enterobacteriaceae and Pseudomonas. Bifidobacteria can reduce an azo compound present in the medium, which gives the colonies a pink-purple coloration.

ABM (ACARYON Bifidobacterium Medium is a modification of the “Bifidobacterium Agar Modified” from BD Catalog Number in May 2019: 254546) contains casein peptone (12 g/l), meat peptone (5 g/l), sodium chloride (5 g/l), beef extract (3 g/l), yeast extract (3 g/l), cornstarch (1 g/l), glucose (2.5 g/l), lactulose (2.5 g/l), cysteine-hydrochloride (0.5 g/l), riboflavin (0.01 g/l) and propionic acid (99%; 5 ml/1). All components were mixed and suspended in distilled water. The pH was adjusted to 5.5+/−0.2 with 5N NaOH.

Modified ABM Nr.1: Meat peptone replaced by soya peptone; modified ABM Nr.2: Beef extract replaced by soya peptone; modified ABM Nr.3: Meat peptone and Beef extract replaced by soya peptone.

It was also found that if Lactobacillus bacteria are cultivated in suboptimal medium (i.e. BSM Broth or ABM Broth) the loading of GM1 or Gb3 was successful even when GM1 or Gb3 was added directly to the cultivating medium. For Stx2: Loading with Gb3 in presence of cholesterol increased the binding of Stx2. Loading in presence of cholesterol alone resulted in no binding at all.

### Example 2: GM1-Loaded Strains Inhibit the Binding of Cholera Toxin to its Natural Receptor (GM1) In Vitro

2.1. Material Methods:

Procedure for testing the in vitro toxin-neutralization capacity of microorganisms loaded with GM1.

GM1 was coated on Maxisorp ELISA plates from Invitrogen and the wells blocks with HSA as mentioned above. Microorganisms loaded with GM1 as described above were diluted in PBS with 2% HSA at OD1 to OD5 and incubated at Room temperature (RT) or 37° C. for 1 hour to prevent any later unspecific binding. GM1 loaded bacteria and labelled CT were given (at different concentrations: e.g. 1 ng/ml to 2 μg/ml and OD600 nm 1 to 10 for CT and GM1 loaded bacteria respectively) at the same time on wells coated with GM1 and incubated at RT or 37° C. for 1 to 2 hours. Labelled CT without GM1-loaded bacteria and labelled CT with unloaded bacteria were used as controls.

The plates were washed, and the extinction measured as mentioned above. Comparing the signal obtained without GM1-loaded bacteria to the signal obtained in presence of GM1-loaded bacteria allowed to quantify the in vitro CT-neutralization capacity of the GM-loaded bacteria.

2.2. Results:

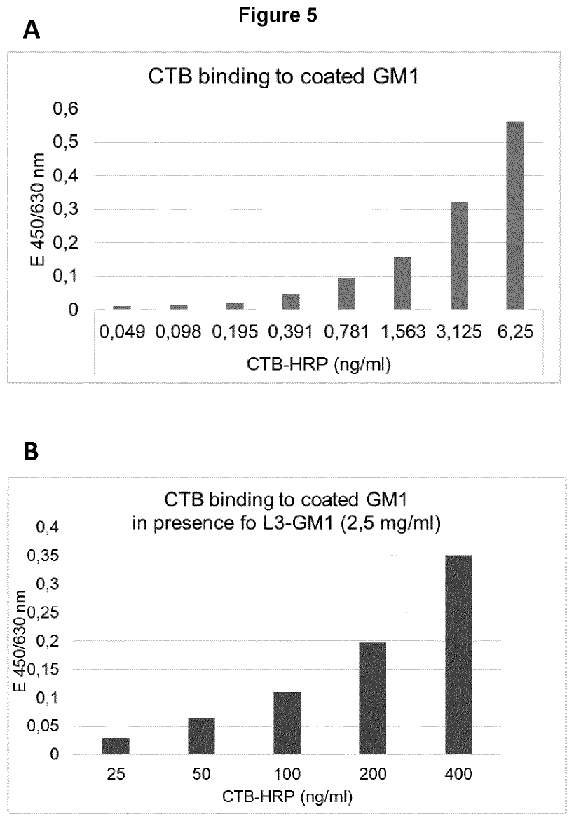

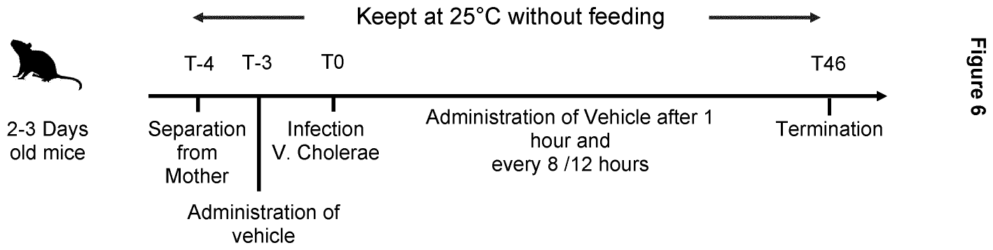

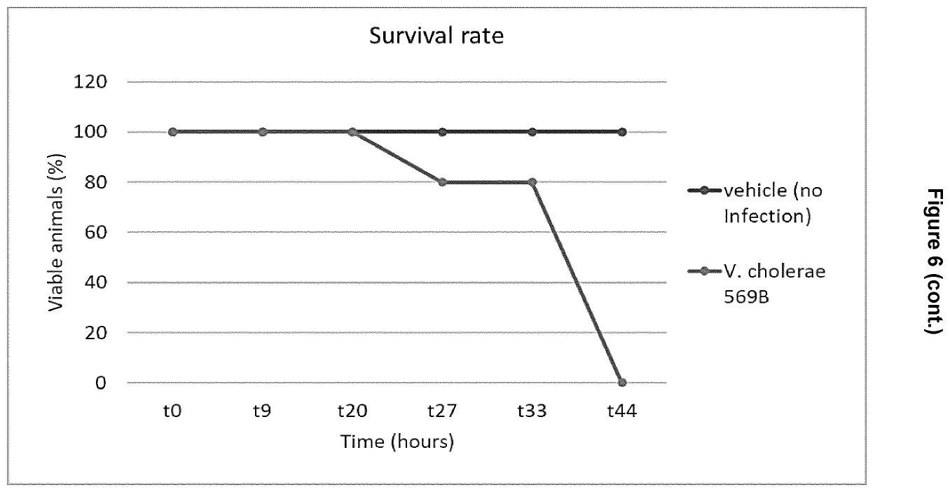

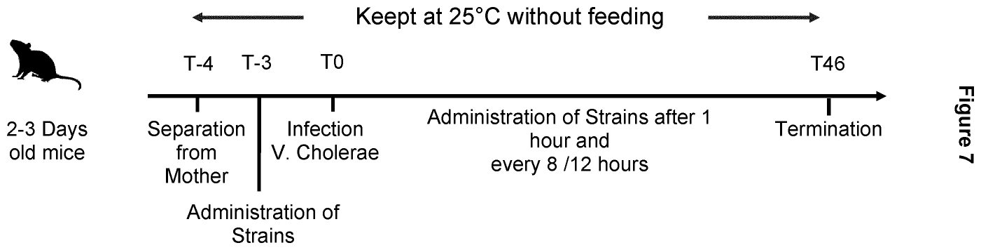

A Lactobacillus strain (L3) was loaded with GM1 (named L3-GM1) as mentioned above and used to assay the in vitro cholera toxin-neutralization capacity as mentioned above too. A signal obtained with 400 ng/ml HRP-CTB+L3-GM1 is equivalent to approx. 4 ng/ml CTB (without L3-GM1), demonstrating that over 99% of the binding of HRP-CTB to GM1 was inhibited by the L3-GM1-strain (2.5 mg/ml) (FIG. 5). Unloaded L3 did not inhibit the binding of labelled-CT to GM1.

### Example 3: In Vivo Experiment 1

3.1. Material and Methods:

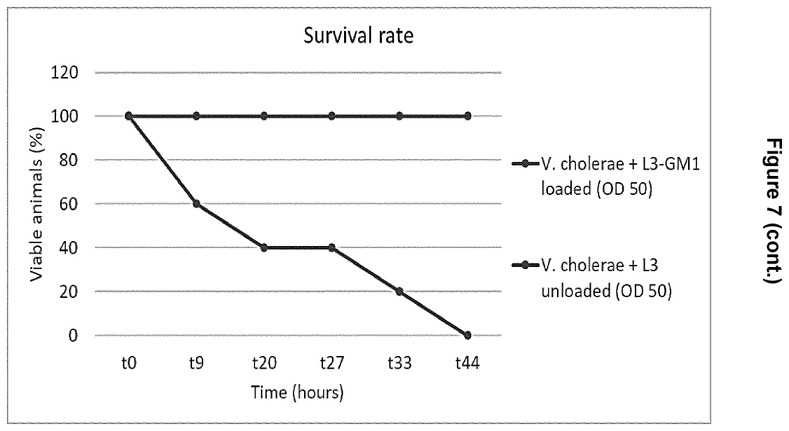

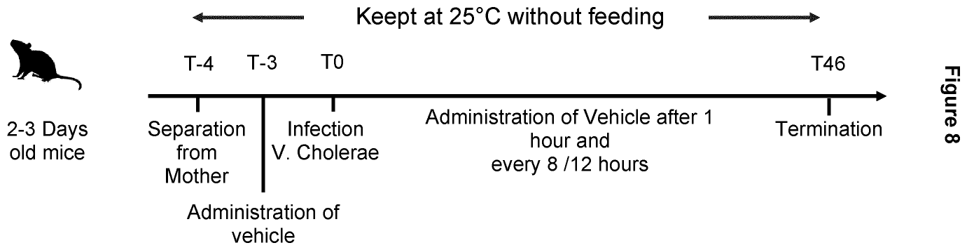

Infant mice (2 to 4 days old) were separated from the mother four hours before infection. Three hours before infection, the mice were gastrically applied with the vehicle or the test strains (L3 or L3-GM1). At infection time, the mice were gastrically applied with 10 times the LD50 dosis of Vibrio cholerae (strain 569B). The mice were further gastrically applied with the vehicle or the test strains one to two hours after infection and every 8 to 12 hours afterward (FIGS. 6 and 7).

Applied dosis of bacterial strain (L3 or L3-GM1): 2.5 mg dry weigth.

3.2. Results:

The survival rate of the control group treated with vehicle, and the test group treated with over 10 times the LD50 dosis of Vibrio cholerae was determined. The last animal of the test group infected with Vibrio cholerae died after approximately 44 hours (FIG. 6).

Mice infected with over 10 times the LD50 dosis of Vibrio cholerae were also applied with the unloaded L3 or the GM1-loaded L3 (L3-GM1) bacterial strains. Animals receiving the CT-binding strain survived the experiment, while animals receiving a CT-non-binding strain died within the experiment (FIG. 7).

### Example 4: In Vivo Experiment 2

4.1. Material and Methods:

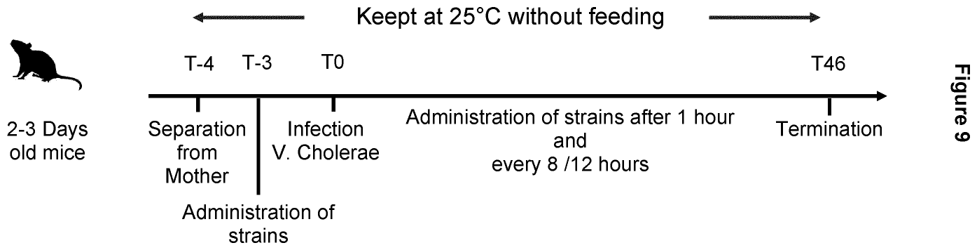

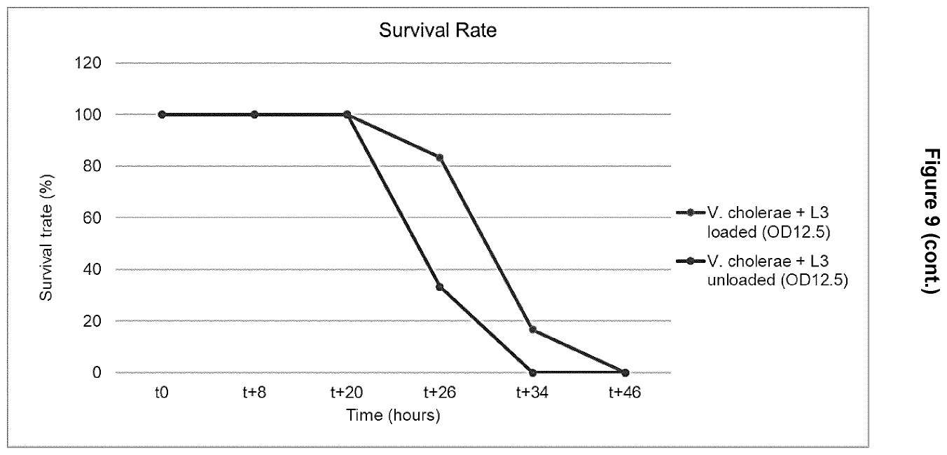

Infant mice (2 to 4 days old) were separated from the mother four hours before infection. Three hours before infection, the mice were gastrically applied with the vehicle or the test strains (L3 or L3-GM1). At infection time, the mice were gastrically applied with 10 times the LD50 dosis of Vibrio cholerae (strain 569B). The mice were further gastrically applied with the vehicle or the test strains one to two hours after infection and every 8 to 12 hours afterward (FIGS. 8 and 9). Applied dose of bacterial strains (L3 or L3-GM1): 0.5 mg dry weight.

4.2. Results:

The survival rate of the control group treated with vehicle, and the test group treated with over 30 times the LD50 dosis of Vibrio cholerae was determined. The last animal of the test group infected with Vibrio cholerae died after approximately 44 hours (FIG. 8).

Mice infected with over 30 times the LD50 dosis of Vibrio cholerae were also applied with a low dose of the unloaded L3 or the GM1-loaded L3 (L3-GM1) bacterial strains. The CT-binding strain presented a beneficial effect in increasing the survival time of the infected animals (FIG. 9).

### Example 5: Stability of the Association Between i.e. a Lipid Carrier and the Bacteria

5.1. Material and Methods:

To test the stability of the association between several microorganisms and GM1 or Gb3, the GM1 or Gb3-loaded bacteria were subjected to different treatments, before being tested for their ability to bind labelled CT or STx as described above. Thus, the loaded cells were subjected to pasteurization (e.g., 15 min at 70° C.), to freeze and taught, to lyophilization, to incubation at pH1 for 1 hour at 37° C., to prolonged incubation at 37° C., to the action of gastric juice for 30 min at 37° C. and storage at 4° C. for 4 weeks.

5.2. Results:

None of the mentioned treatment, induced a reduction of the CT-binding of the GM1-loaded Bacteria of more than 20%.

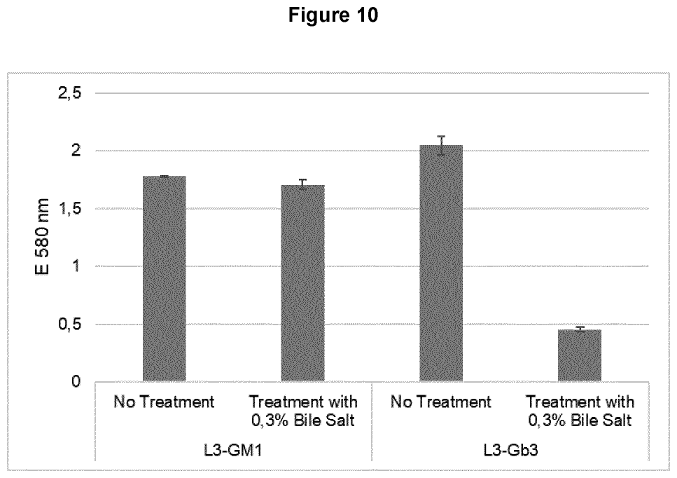

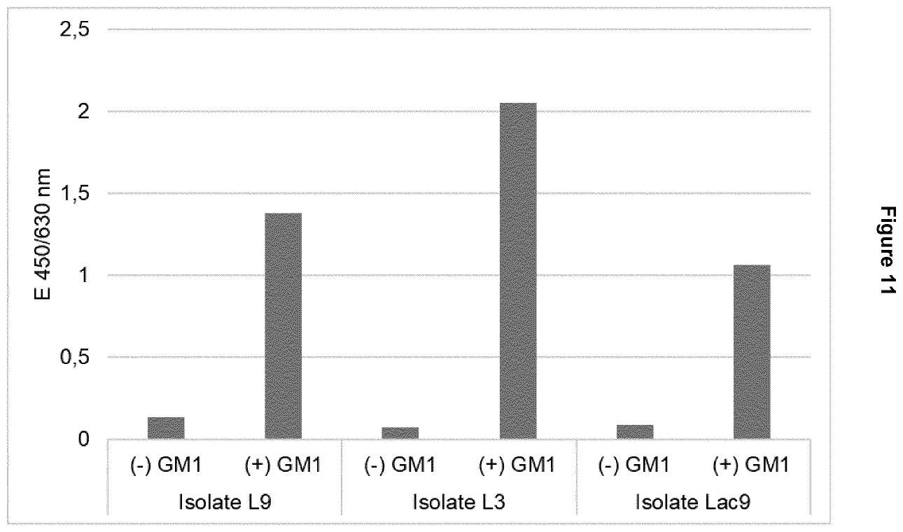

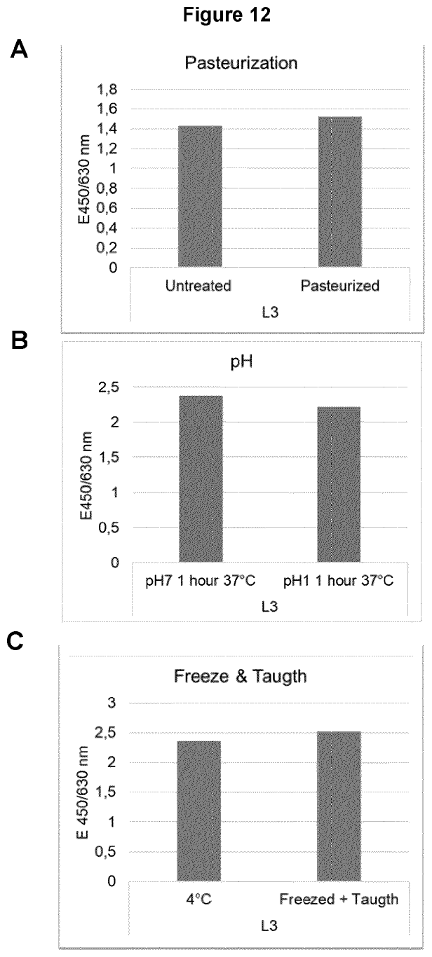

The association between Gb3 and the loaded bacteria presented similar stability except against treatment with gut juice or 0.3% bile salt (FIG. 10).

### Example 6: Specific Growth, Treatment and Loading Conditions to Stabilize the Microorganism-Lipid Carrier Interaction

6.1. Material and Methods:

Test resistance to treatment with bile salt 0.3% (in PBS) for 1 hour at 37° C.

Loaded microorganisms are washed and resuspended at OD 8 in 0.3% bile salt (in PBS) and incubated for 1 hour at 37° C. After treatment with bile salt 0.3% these microorganisms are washed three times with PBS or DPBS before being tested for the presence of the lipid-carrier on their surface as mentioned above.

Loading conditions.

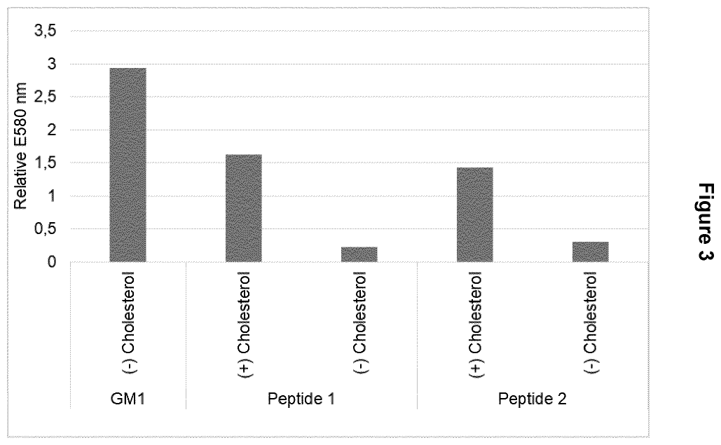

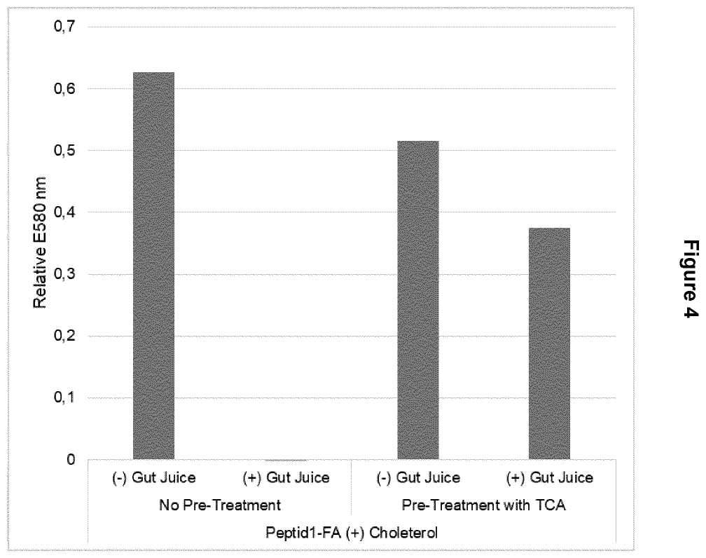

Bacteria (Lactobacillus paracasei L3) were treated as depicted in FIG. 2. In brief the bacteria were washed before being incubated overnight in ABM at 37° C. under anaerobic conditions with 3 μg/ml of Gb3 and 1% cholesterol. To assess the stability of the association, loaded cells were incubated with Ileum juice containing 0.3% bile salt at 37° C. for one hour (right bars). Control cells were incubated in PBS at 37° C. for one hour (left bars). The presence of the receptor on the surface of the isolates was analyzed by ELISA using Stx1-AP toxin.

6.2. Results:

Special culture conditions and cholesterol and pre-treatment with TCA do stabilize the association of Gb3. All other tested pre-treatment did not stabilize the association (no right bars to be seen) (FIG. 2).

### Example 7: Loading Protocol and Treatment Conditions for Stable Association of Gb3

In summary the following culture, pre-treatment, and loading protocol were found.

7.1. Material and Methods:

Loading protocols.

Preferred culture, pre-treatment, loading conditions and stability test Table 2:

| | | | | | Composition |
|---|---|---|---|---|---|
| Strain | Culture | Treatment | Loading | Stability | of Pancreatin Juice |
| Lactobacillus | Initial Inoculation in | Treatment | Inoculate | Resist treatment | for 50 ml: |
| paracasei | MRS (anaerobic | in 10% | in ABM (Final | with pancreatin | 340 mg |
| (L3) | conditions) and | TCA at | OD 1) and add | juice for 1 hour | KH₂PO₄/150 mg |
| | growth O/N at 37° C. - | 90° C. | 3 μg/ml Gb3 - | at 37° C. as | bile salts |
| | Further inoculation | for 15 | Incubation | described in | (0.3%)/62.5 mg |
| | (at 5%) in ABM | min. | under anaerobic | mat and met, | Pancreatic/ |
| | (anaerobic conditions) | | conditions at | | 3.8 ml NOH/ |
| | and growth for | | 37° C. O/N | | (pH 6.8) |
| | 3 days at 37° C. | | | | |
| Lactobacillus | Initial Inoculation in | Treatment | Inoculate | | |
| paracasei | MRS (anaerobic | in 10% | in ABM (Final | | |
| (L3) | conditions) and | TCA at | OD 1) and add | | |
| | growth O/N at 37° C. - | 90° C. | 3 μg/ml Gb3 + | | |
| | Further inoculation | for 15 | 1% Cholesterol - | | |
| | (at 5%) in ABM | min. | Incubation | | |
| | (anaerobic conditions) | | under anaerobic | | |
| | and growth for | | conditions at | | |
| | 3 days at 37° C. | | 37° C. for 3 days | | |
| Lactobacillus | Initial Inoculation in | Treatment | Inoculate | | |
| paracasei | MRS (anaerobic | in 10% | in ABM (Final | | |
| (L3) | conditions) and | TCA at | OD 1) and add | | |
| | growth 2 days at 37° C. - | 90° C. | 3 μg/ml Gb3 +/− | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. | | 37° C. for 3 days | | |
| Lactobacillus | Initial Inoculation in | No | Inoculate | | |
| paracasei | MRS (anaerobic | Treatment | in ABM (Final | | |
| (L3) | conditions) and | | OD 1) and add | | |
| | growth 2 days at 37° C. - | | 3 μg/ml Gb3 +/− | | |
| | Further inoculation | | 1% Cholesterol - | | |
| | (at 1%) in MRS | | Incubation | | |
| | (anaerobic conditions) | | under anaerobic | | |
| | and growth for | | conditions at | | |
| | 1 days at 37° C. | | 37° C. for 3 days | | |
| Lactobacillus | Initial Inoculation in | Treatment | Inoculate | | |
| paracasei | MRS (anaerobic | in 10% | in ABM (Final | | |
| (L3) | conditions) and | TCA at | OD 1) and add | | |
| | growth 2 days at 37° C. - | 90° C. | 3 μg/ml Gb3 +/− | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. | | 37° C. for 3 days | | |
| Lactobacillus | Initial Inoculation in | Treatment | Inoculate | | |
| paracasei | MRS (anaerobic | with | in ABM (Final | | |
| (L3) | conditions) and | pancreatin | OD 1) and add | | |
| | growth 2 days at 37° C. - | juice 1 | 3 μg/ml Gb3 +/− | | |
| | Further inoculation | hour at | 1% Cholesterol - | | |
| | (at 1%) in MRS | 37° C. | Incubation | | |
| | (anaerobic conditions) | | under anaerobic | | |
| | and growth for | | conditions at | | |
| | 1 days at 37° C. | | 37° C. for 3 days | | |
| Lactobacillus | Initial Inoculation in | Treatment | Inoculate | | |
| paracasei | MRS (anaerobic | in 10% | in ABM (Final | | |
| (L3) | conditions) and | TCA at | OD 1) and add | | |
| | growth 2 days at 37° C. - | 90° C. | 3 μg/ml Gb3 + | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. - | | 37° C. for 3 days | | |
| | Further inoculation | | | | |
| | (at 1%) in MRS | | | | |
| | (anaerobic conditions) | | | | |
| | and growth for | | | | |
| | 3 days at 37° C. | | | | |
| Lactobacillus | Initial Inoculation in | Treatment | Inoculate | | |
| paracasei | MRS (anaerobic | in 10% | in ABM (Final | | |
| (L3) | conditions) and | TCA at | OD 1) and add | | |
| | growth 2 days at 37° C. - | 90° C. | 3 μg/ml Gb3 + | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. - | | 37° C. for 3 days | | |
| | Further inoculation | | | | |
| | (at 1%) in MRS | | | | |
| | (anaerobic conditions) | | | | |
| | and growth for | | | | |
| | 3 days at 30° C. | | | | |
| Lactobacillus | Initial Inoculation in | Treatment | Inoculate | | |
| paracasei | MRS (anaerobic | in 10% | in ABM (Final | | |
| (L3) | conditions) and | TCA at | OD 1) and add | | |
| | growth 2 days at 37° C. - | 90° C. | 3 μg/ml Gb3 + | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. - | | 37° C. for 3 days | | |
| | Further inoculation | | | | |
| | (at 1%) in ABM | | | | |
| | (anaerobic conditions) | | | | |
| | and growth for | | | | |
| | 3 days at 37° C. | | | | |
| Bifidobacterium | Initial Inoculation in | Treatment | Inoculate | | |
| animalis | ABM (anaerobic | in 10% | in ABM (Final | | |
| (B1) | conditions) and | TCA at | OD 1) and add | | |
| | growth O/N at 37° C. - | 90° C. | 3 μg/ml Gb3 + | | |
| | Further inoculation | for 15 | 1% Cholesterol - | | |
| | (at 5%) in ABM | min. | Incubation | | |
| | (anaerobic conditions) | | under anaerobic | | |
| | and growth for | | conditions at | | |
| | 1 days at 37° C. | | 37° C. for 3 days | | |
| Bifidobacterium | Initial Inoculation in | Treatment | Inoculate | | |
| animalis | ABM (anaerobic | in 10% | in ABM (Final | | |
| (B1) | conditions) and | TCA at | OD 1) and add | | |
| | growth O/N at 37° C. - | 90° C. | 3 μg/ml Gb3 + | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. | | 37° C. for 3 days | | |
| Enterococcus | Initial Inoculation in | Treatment | Inoculate | | |
| Faeclis | ABM (anaerobic | in 10% | in ABM (Final | | |
| (7) | conditions) and | TCA at | OD 1) and add | | |
| | growth O/N at 37° C. - | 90° C. | 3 μg/ml Gb3 + | | |
| | Further inoculation | for 15 | 1% Cholesterol - | | |
| | (at 5%) in ABM | min. | Incubation | | |
| | (anaerobic conditions) | | under anaerobic | | |
| | and growth for | | conditions at | | |
| | 1 days at 37° C. | | 37° C. for 3 days | | |
| Enterococcus | Initial Inoculation in | Treatment | Inoculate | | |
| Faeclis | ABM (anaerobic | in 10% | in ABM (Final | | |
| (12) | conditions) and | TCA at | OD 1) and add | | |
| | growth O/N at 37° C. - | 90° C. | 3 μg/ml Gb3 + | | |
| | Further inoculation | for 15 | 1% Cholesterol - | | |
| | (at 5%) in ABM | min. | Incubation | | |
| | (anaerobic conditions) | | under anaerobic | | |
| | and growth for | | conditions at | | |
| | 1 days at 37° C. | | 37° C. for 3 days | | |
| Enterococcus | Initial Inoculation in | Treatment | Inoculate | | |
| Faeclis | ABM (anaerobic | in 10% | in ABM (Final | | |
| (7) | conditions) and | TCA at | OD 1) and add | | |
| | growth O/N at 37° C. - | 90° C. | 3 μg/ml Gb3 + | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. | | 37° C. for 3 days | | |
| Lactobacillus | Initial Inoculation in | Treatment | Inoculate | | |
| paracasei | MRS (anaerobic | in 10% | in ABM (Final | | |
| (L3) | conditions) and | TCA at | OD 1) and add | | |
| | growth 1 days at 37° C. - | 90° C. | 3 μg/ml Gb3 + | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. | | 37° C. for 1 days | | |
| Lactobacillus | Initial Inoculation in | Treatment | Inoculate | | |
| paracasei | MRS (anaerobic | in TCA 5 | in ABM (Final | | |
| (L3) | conditions) and | to 25% at | OD 1) and add | | |
| | growth 1 days at 37° C. - | 90° C. | 3 μg/ml Gb3 + | | |
| | Further inoculation | for 15 | 1% Cholesterol - | | |
| | (at 1%) in MRS | min. | Incubation | | |
| | (anaerobic conditions) | | under anaerobic | | |
| | and growth for | | conditions at | | |
| | 1 days at 37° C. | | 37° C. for 3 days | | |

Even more preferred culture, pre-treatment, loading conditions and stability test are depicted in Table 3:

| Strain | Culture | Treatment | Loading | Stability |
|---|---|---|---|---|
| Lactobacillus | Initial Inoculation | Treatment | Inoculate | Resist treatment |
| paracasei | in MRS (anaerobic | in 10% | in ABM (Final | with 0.3% bile salt |
| (L3) | conditions) and | TCA at | OD 1) and add | and/or pancreatin for |
| | growth O/N at 37° C. - | 90° C. | 3 μg/ml Gb3 - | 1 hour at 37° C. |
| | Further inoculation | for 15 | Incubation | as described in |
| | (at 5%) in ABM | min. | at 37° C. O/N | mat and met, |
| | (anaerobic conditions) | | or 3 days | |
| | and growth for | | | |
| | 3 days at 37° C. | | | |
| Lactobacillus | Initial Inoculation | Treatment | Inoculate | |
| paracasei | in MRS (anaerobic | in 10% | in ABM (Final | |
| (L3) | conditions) and | TCA at | OD 1) and add | |
| | growth 2 days at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 3 days | |
| | 1 days at 37° C. | | | |
| Lactobacillus | Initial Inoculation | | Inoculate | |
| paracasei | in MRS (anaerobic | | in ABM (Final | |
| (L3) | conditions) and | | OD 1) and add | |
| | growth 2 days at 37° C. - | | 3 μg/ml Gb3 +/− | |
| | Further inoculation | | 1% Cholesterol - | |
| | (at 1%) in MRS | | Incubation | |
| | (anaerobic conditions) | | at 37° C. | |
| | and growth for | | for 3 days | |
| | 1 days at 37° C. | | | |
| Lactobacillus | Initial Inoculation | Treatment | Inoculate | |
| paracasei | in MRS (anaerobic | in 10% | in ABM (Final | |
| (L3) | conditions) and | TCA at | OD 1) and add | |
| | growth 2 days at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 3 days | |
| | 1 days at 37° C. - | | | |
| | Further inoculation | | | |
| | (at 1%) in MRS | | | |
| | (anaerobic conditions) | | | |
| | and growth for | | | |
| | 3 days at 37° C. | | | |
| Lactobacillus | Initial Inoculation | Treatment | Inoculate | |
| paracasei | in MRS (anaerobic | in 10% | in ABM (Final | |
| (L3) | conditions) and | TCA at | OD 1) and add | |
| | growth 2 days at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 3 days | |
| | 1 days at 37° C. - | | | |
| | Further inoculation | | | |
| | (at 1%) in MRS | | | |
| | (anaerobic conditions) | | | |
| | and growth for | | | |
| | 3 days at 30° C. | | | |
| Lactobacillus | Initial Inoculation | Treatment | Inoculate | |
| paracasei | in MRS (anaerobic | in 10% | in ABM (Final | |
| (L3), | conditions) and | TCA at | OD 1) and add | |
| | growth 2 days at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 3 days | |
| | 1 days at 37° C. - | | | |
| | Further inoculation | | | |
| | (at 1%) in ABM | | | |
| | (anaerobic conditions) | | | |
| | and growth for | | | |
| | 3 days at 37° C. | | | |
| Bifidobacterium | Initial Inoculation | Treatment | Inoculate | |
| animalis | in ABM (anaerobic | in 10% | in ABM (Final | |
| (B1) | conditions) and | TCA at | OD 1) and add | |
| | growth O/N at 37° C. - | 90° C. | 3 μg/ml Gb3 + | |
| | Further inoculation | for 15 | 1% Cholesterol - | |
| | (at 5%) in ABM | min. | Incubation | |
| | (anaerobic conditions) | | at 37° C. | |
| | and growth for | | for 3 days | |
| | 1 days at 37° C. | | | |
| Bifidobacterium | Initial Inoculation | Treatment | Inoculate | |
| animalis | in ABM (anaerobic | in 10% | in ABM (Final | |
| (B1) | conditions) and | TCA at | OD 1) and add | |
| | growth O/N at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 3 days | |
| | 1 days at 37° C. | | | |
| Enterocoocus | Initial Inoculation | Treatment | Inoculate | |
| Faeclis | in ABM (anaerobic | in 10% | in ABM (Final | |
| (EF7; EF12) | conditions) and | TCA at | OD 1) and add | |
| | growth O/N at 37° C. - | 90° C. | 3 μg/ml Gb3 + | |
| | Further inoculation | for 15 | 1% Cholesterol - | |
| | (at 5%) in ABM | min. | Incubation | |
| | (anaerobic conditions) | | at 37° C. | |
| | and growth for | | for 3 days | |
| | 1 days at 37° C. | | | |
| Enterocoocus | Initial Inoculation | Treatment | Inoculate | |
| Faeclis | in ABM (anaerobic | in 10% | in ABM (Final | |
| (EF7; EF12) | conditions) and | TCA at | OD 1) and add | |
| | growth O/N at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 3 days | |
| | 1 days at 37° C. | | | |
| Lactobacillus | Initial Inoculation | Treatment | Inoculate | |
| paracasei | in MRS (anaerobic | in 10% | in ABM (Final | |
| (L3) | conditions) and | TCA at | OD 1) and add | |
| | growth 1 days at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 1 days | |
| | 1 days at 37° C. | | | |
| Lactobacillus | Initial Inoculation | Treatment | Inoculate | |
| paracasei | in MRS (anaerobic | in 10% | in ABM (Final | |
| (L3) | conditions) and | TCA at | OD 1) and add | |
| | growth 1 days at 37° C. - | 90° C. | 3 μg/ml Gb3 + | |
| | Further inoculation | for 15 | 1% Cholesterol - | |
| | (at 1%) in MRS | min. | Incubation | |
| | (anaerobic conditions) | | at 37° C. | |
| | and growth for | | for 3 days | |
| | 1 days at 37° C. | | | |

7.2. Results:

A loading under the conditions described in Table 3 allowed the best association of the lipid carrier that resisted the stability test.

### Example 8: Association of Lipopeptides

Two GM1-mimicry peptides (described by Robert K Yu et al. Glycobiology. 2016 January; 26(1): 63-73.) were used. Both peptides were modified through association with a fatty acid (stearic acid) on the N-terminal side of the peptide (FIG. 3).

8.1. Material and Methods:

Bacteria (Lactobacillus paracasei L3) were incubated overnight in SBSM/ABM medium at 37° C. under anaerobic conditions with 10 μg/ml of FA-Peptide 1 or FA-Peptide 2 or GM1 with or without 1% cholesterol. The presence of GM1 or the Peptide on the surface of the isolates was analyzed by ELISA using CT-AP as mentioned above.

8.2. Results:

Loading was only possible in presence of cholesterol in the loading medium (FIG. 3). Both GM1-mimicry peptides were demonstrated to bind CT-AP (alkalin phosphatase labeled cholera toxin) but not Stx1-AP (alkalin phosphatase labelled shiga toxin 1), demonstrating the specificity of the binding in ELISA. Furthermore, both modified peptides were able to partially inhibit the binding of CT-AP to its natural receptor GM1.

### Example 9: Stability of Lipopeptides

The described lipopeptide association was not stable when conducted under standard loading conditions. Thus, when incubated with Ileum juice containing 0.3% bile salt at 37° C. for one hour, the CT-AP binding capacity of the loaded L3-cells was lost (FIG. 4).

Incubating the microorganisms in 10% TCA at 90° C. for 15 minutes before the loading incubation resulted in a stable association that resisted the bile salt treatment (FIG. 4).

9.1. Material and Methods:

Bacteria (Lactobacillus paracasei L3) were incubated overnight in SBSM/ABM at 37° C. under anaerobic conditions with 10 μg/ml of FA-Peptide 1 with 1% cholesterol. The presence of the peptide on the surface of the isolates was analyzed by ELISA using AP-cholera toxin (alkaline phosphatase labeled cholera toxin).

To assess the stability of the association, loaded cells were incubated with Ileum juice containing 0.3% bile salt at 37° C. for one hour. Control cells were incubated in PBS at 37° C. for one hour ((−) Gut Juice).

9.2. Results:

Using the Lactobacillus paracasei isolate L3, the following cultivation process ensured a stable association without need for TCA treatment: 1) inoculation in MRS and growth 0/N at 37° C. under anaerobic conditions. 2) further inoculation at 5% in MRS and cultivation for 16 hours at 37° C. under anaerobic conditions. 3) Loading in modified SBSM or ABM-Medium for 1 to 3 days at 37° C. under anaerobic conditions.

Example 10: Isolation method for suitable microorganism (i.e. Gb3 molecule as a receptor for the Shiga Toxin (Stx).

Beside using the standard loading protocols mentioned above, a microorganism with specific properties may be needed for some specific application and/or lipid-carrier (i.e. a microorganism that naturally initiated a stable interacting with Gb3).

10.1. Material and Methods:

Coupling the Cholera Toxin to magnet beads.

Preparation of beads.

Dynabeads® M-270 Amine (Life technologies, UK) were used to directly link the Cholera toxin (List Biological Laboratories, Inc). The Surface-reactive primary amino-groups allow immobilization of ligands such as carbohydrates, glycoproteins and glycolipids through reductive amination of aldehyde or ketone groups. Alternatively, ligands can be immobilized through amide-bond formation with carbodiimide-activated carboxylic acid groups. Bi-functional cross-linkers may be used to introduce other functional groups.

25 μl of Dynabeads® M-270 Amine were placed in 1.5 ml Safe-Lock Eppendorf tubes (Eppendorf, Hamburg, Germany), washed twice with 100 μl buffer F (0.26 g NaH2PO4+H₂O, 1.44 g Na2HPO4+2H₂O, 8.78 g NaCl, pH 7.4) using the Dynal Magnetic Particle Concentrator®-S(MPC®-S, Dynal Biotech, Oslo, Norway) and suspended in 25 μl buffer F. Before coupling of ligands to Beads the washed Dynabeads were activated with NHS (N-hydroxy-succinimidyl)-ester cross-Linker. Hereby the beads were re-suspended in 0.1 M sodium phosphate buffer with 0.15 M NaCl, pH 7.4. Dissolved NHS-ester (15 mg/ml) were added to the bead-solution. Beads were then incubated for 30 min at room temperature with slow tilt and rotation, and finally washed twice with buffer F and re-suspended in 25 μl buffer F. Shiga Toxin were then dissolved in water. To eliminate amino groups (Tris-) present in the initial buffer, shiga toxin solution were dialyzed against PBS.

Preparation and Gb3-loading of samples.

1 g of a mixture of microorganisms (e.g., fresh feces sample from healthy adults) were suspended in a tube containing 9 ml of anaerobic PBSred (8.5 g/l NaCl, 0.6 g/l Na2HPO4, 0.3 g/l KH2PO4, 0.25 g/l Cystein.HCl, 0.1 g/l Peptone, pH 7.0) and stored at 4° C. in an anaerobic box (Anaerogen, Oxoid, Wesel, Germany). The samples were then processed as follows: sterile 3 mm diameter glass beads were added, and the samples homogenized by vortexing. The homogenized fecal suspensions were centrifuged (300×g for 1 min) to sediment debris. The resulting supernatants were transferred in new tubes and centrifuged again. The supernatants were diluted 1:100 (v/v) in different growth media (i.e., LB broth, MRS broth and WC broth) and incubated overnight at 37° C. under anaerobic conditions. The cultures were washed with PBS, resuspended in anaerobic PBS and used directly or frozen at −20° C. for later use. The cultures in PBS were set to an OD1 to 10 added with 1 to 10 μg/ml of Gb3 (Gerbu) with or without addition of 1% cholesterol and incubated at 30° C. or 37° C. overnight. The cells were than extensively washed with PBS before being resuspended in PBSred 0.1% BSA.

Optionally, the fecal sample may be depleted or enriched for any specific genus or species of microorganisms before loading, e.g., by mean of affinity depletion/enrichment, antibiotic treatment or any suitable alternative method known in the art. For example, in order to increase the proportion of Bifidobacterium and Lactobacillus microorganisms (bacteria) in fecal samples, the samples were further depleted for Bacteroides (bacteria) by the means of centrifugations. Bacteroides are the most abundant genus of the human colonic microbiota (i.e., microflora), surpassing Lactobacillus and Bifidobacterium by a factor of 10.000. In order to increase the proportion of Bifidobacterium and Lactobacillus bacteria in fecal samples, the feces suspensions were centrifuged for 3 min at 2500 rpm. Due to their small size, Bacteroides were mostly restricted to the supernatant that could be discarded or used for further experiments. The pellets were resuspended in PBSred and the procedure was repeated three times. The final bacteria pellets were loaded with GM1 as described above.

Optionally the loaded sample may be subject to any relevant treatment that may help the isolation of microorganism with specific properties. E.g. if microorganisms are needed that do interact with the sphingolipid (as described in the application) in such a way that the interaction resists the action of 0.3% bile salt, the sample may be treated with 0.3% bile salt the ensure that all microorganism-sphingolipid (e.g. Gb3) interactions that do not resist the treatment are removed before the isolation. After the treatment, the sample may be washed to remove excess of free sphingolipids. Only microorganism-sphingolipid interactions that resisted the treatment are still available.

Isolation of microorganisms naturally binding Gb3 and presenting the glycosylic moiety to the environment (i.e., on their exterior surface).

Isolation of bacteria using toxin-coated beads.

20 μl of Gb3-loaded samples were added to 175 μl PBSred+0.1% HSA and 5 μl “Cholera toxin”-coated beads. The mixes were incubated at a suitable temperature (e.g., room temperature (RT) or 37° C.) under appropriate atmosphere and gentle agitation for 1 hour. Subsequently, the beads were washed twice with 200 μl PBSred+0.01% BSA. To reduce non-specific binding of viable bacteria, the beads were pre-incubated for 1 hour with pasteurized bacteria (e.g., at RT or 37° C.). Pasteurized bacteria may also be added during the incubation of viable bacteria with the toxin-coated beads.

After incubation the beads were washed and resuspended in 1 ml PBSred. 100 μl aliquots were spread-plated on unspecific and specific agar plates like, e.g., WC agar (Oxoid, Wesel, Germany), MRS agar (Roth, Karlsruhe, Germany), Bifidus Selective agar (BSM, Sigma Aldrich, Taufkirchen, Germany) and ACARYON Bifidobacterium Selective agar (ABM Agar).

WC Medium: Tryptone 10 g/L; Gelatin peptone 10 g/L; Yeast extract 5 g/L; Glucose 1 g/L; Sodium chloride 5 g/L; L-Arginine 1 g/L; Sodium pyruvate 1 g/L; Menadione 0.0005 g/L; Haemin 0.005 g/L (pH=7±0.2).

WC Agar: Tryptone 10 g/L; Gelatin peptone 10 g/L; Yeast extract 5 g/L; Glucose 1 g/L; Sodium chloride 5 g/L; L-Arginine 1 g/L; Sodium pyruvate 1 g/L; Menadione 0.0005 g/L; Haemin 0.005 g/L; Agr 10 g/L (pH=7±0.2).

MRS Agar: Peptone 10 g/l; Yeast extract 4 g/l; Beef extract 8 g/l; Glucose 20 g/l; Dipotassium phosphate 2 g/l; Sodium acetate 5 g/l; Ammonium citrate 2 g/l; Magnesium sulphate (MgSO₄) 0.2 g/l; Manganese sulphate 0.05 g/l; Polysorbate 80 1 g/l; Agar 10 g/l; pH=6.2±0.2.

BSM Agar: (Sigma Aldrich Catalog Number in May 2019: 88517). Principle and Interpretation: BSM contains Peptone and Meat extract as sources of carbon, nitrogen, vitamins and minerals. Yeast extract supplies B-complex vitamins which stimulate bacterial growth. Dextrose is the carbohydrate source. Sodium chloride maintains the osmotic balance. There is a compound in low concentration for detoxify metabolic by-products. The medium contains reducing and buffering agents. Selective salts inhibit the growth of molds, Enterococci and other Gram-negative bacteria. Another compound inhibits glycolysis by inactivating glyceraldehyde-3-phosphate dehydrogenase present and important in different bacteria and fungi (also Streptococci sp.). Three antibiotics are the selective agents and inhibit the accompanying bacterial flora like Bacilli, Enterobacteriaceae and Pseudomonas. Bifidobacteria that can reduce an azo compound present in the medium, which gives the colonies a pink-purple coloration.

ABM Agar contains casein peptone (12 g/l), meat peptone (5 g/l), sodium chloride (5 g/l), beef extract (3 g/l), yeast extract (3 g/l), cornstarch (1 g/l), glucose (2.5 g/l), lactulose (2.5 g/l), cysteine-hydrochloride (0.5 g/l), riboflavin (0.01 g/l), propionic acid (99%; 5 ml/1) and Agar 10 g/l. All components were mixed and suspended in distilled water. The pH was adjusted to 5.5+/−0.2 with 5N NaOH.

All plates were incubated under suitable conditions (e.g., Table 1). Well isolated colonies were picked randomly from agar plates. Optionally, the colonies may be streaked several times on nonselective or selective media. Alternatively, isolated colonies may be pooled together and submitted again (several times) to the isolation process.

Growth and maintenance of isolates.

Well separated colonies were randomly picked from agar plates, inoculated into corresponding broth mediums and grown under suitable conditions. The resulting cultures may partly be used for production of cryo-stocks and partly used for future screening analysis. For screening analysis, bacteria cultures were centrifuged, washed with PBS and re-suspended in PBS. Alternatively, bacteria cultures were further cultivated in suboptimal medium (that does not promote culture growth) or in a medium containing propionic acid at 1 to 10 ml/l. Such bacterial cultures were than centrifuged, washed with PBS and re-suspended in PBS.

Identification of isolates.

Preliminary identification of isolated bacteria was based on microbiological analysis (e.g., Gram staining and microscopic analysis) and biochemical analysis (e.g. with rapid ID 32A biochemical test kits (BioMérieux, Marcy l′Etoile, France)).

Alternatively, the characterization was performed by Bruker Biotyper (version 2.0) matrix-assisted laser desorption ionization—time of flight (MALDI-TOF) mass spectrometry. Briefly, colonies were directly picked and applied as a thin film onto a polish steel plate and allowed to dry at room temperature. Subsequently, 1 μl of MALDI matrix (Bruker Daltonics) in 50% acetonitrile and 2.5% trifluoroacetic acid was applied and allowed to dry again.

For the extraction method, 1 to 2 colonies (or a few colonies in the case of a small colony size) were suspended in 300 μl of molecular-grade water (Sigma-Aldrich, St. Louis, Mo.) and vortexed. Next, 900 μl of 100% ethanol (Sigma-Aldrich) was added, vortexed, and centrifuged (13,400×g) for 2 min. The supernatant was decanted, and the pellet was dried at room temperature. 10 μl of 70% formic acid (Fluka [Sigma-Aldrich], St. Louis, Mo.) and 10 μl of acetonitrile (Fluka) were added and thoroughly mixed by pipetting, followed by centrifugation (13,400×g) for 2 min. One microliter of supernatant was spotted onto the 384-spot plate and allowed to dry at room temperature before the addition of 1 μl of matrix. For each plate, a bacterial test standard (Bruker Daltonics) was included to calibrate the instrument and validate the run. MALDI-TOF MS was performed with the MicroFlex LT mass spectrometer (Bruker Daltonics) according to the manufacturer's suggested recommendations. Identification score criteria used were those recommended by the manufacturer: a score of 2,000 indicated species-level identification.

10.2. Results:

GM1 binding capacity of selected isolates.

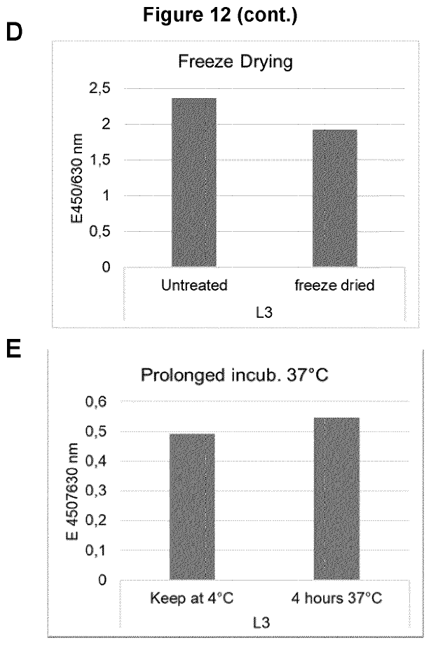

To test the ability of selected bacterial isolates to incorporate the Glycolipid GM1 and to present the oligosaccharide moiety to the environment (e.g., on the exterior cell surface), said isolates were loaded and incubated with as tested through their ability after loading to bind HRP-cholera toxin. This was tested by the means of ELISA. As presented in FIG. 11, the HRP-cholera toxin bound to several isolates after those isolates have been loaded with GM1.

Characterization of the Strains.

The strains designated as “L3” were characterized as belonging to the species Lactobacillus paracasei, the strains designated as “L9” and “Lac9” to the species Lactobacillus reuteri.

Stability of the association with GM1.

The stability of the association between the bacteria and GM1 was assayed for the strain L3 by means of ELISA using labelled Cholera Toxin after pre-treatment of the loaded bacteria as described in material and methods. As presented in FIG. 12, none of the pre-treatment was accompanied by the loss of the bound GM1 suggesting that the binding is stable.

Loading is Independent on the Carbohydrate Moiety of GM1.

To identify which part of the GM1 molecule is involved in the interaction with our bacterial strain L3, the strain was loaded as described in material and methods with either GM1 or GM1a (the carbohydrate part of GM1). The presence of the Carbohydrate moiety was analyzed by ELISA using HRP-CT. In contrast to the strong signal observed with bacteria loaded with GM1, no signal could be obtained with bacteria loaded with GM1a. This result demonstrates that the interaction between GM1 and the bacteria involves the sphingosine and/or fatty acid part of the GM1 molecule.

1-30. (canceled)

31. A modified microorganism comprising:
i) a cell, wherein said cell does not comprise a mycomembrane and
ii) a heterologous lipid carrier, said lipid carrier comprising:
a) a lipid portion, wherein said lipid portion is at least partially associated with an exterior surface of said cell, wherein said lipid portion comprises one or both a ceramide-like glycolipid moiety and a fatty acid moiety; and
b) a non-lipid portion, wherein said microorganism is capable of one or both locating and displaying said non-lipid portion or fragment thereof onto the exterior surface of said cell;
and wherein said heterologous lipid carrier is not alpha-galactosylceramide.

32. The modified microorganism of claim 31, wherein one or more of:
(a) said exterior surface of said cell comprises: a cell wall and/or a cell membrane and/or an outer cell membrane and/or a polysaccharide;
(b) said lipid portion is at least partially incorporated and/or adhered and/or bound to said exterior surface of said cell;
(c) said non-lipid portion comprising a first carbohydrate moiety, a lipopeptide moiety, a linker, a chemical compound, and a first peptide moiety;
(d) said modified microorganism further comprises a heterologous steroid moiety selected from the group consisting of cholesterol, a cholesterol derivative and a cholesterol analog thereof; and
(e) wherein said mycomembrane is located in the exterior surface of said cell of said modified microorganism.

33. The modified microorganism of claim 31, wherein one or both of (a) the lipid-portion further comprises an amino alcohol moiety; and (b) the first carbohydrate of said non-lipid portion contains a sialic acid residue.

34. The modified microorganism of claim 33, wherein said amino alcohol moiety is sphingosine.

35. The modified microorganism of claim 31, wherein the association of the heterologous lipid carrier with said exterior surface of said cell one or both of (a) resists treatment with 0.3% bile salts, and (b) remains associated after treatment with 0.3% bile salts.

36. The modified microorganism of claim 35, wherein said treatment further comprises said 0.3% bile salts in combination with pancreatine juice in Phosphate Buffered Saline (PBS) or Dulbecco's Phosphate Buffered Saline (DPBS) for at least 1 hour at least at 37° C.

37. The modified microorganism of claim 31, has one or more of the following characteristics:
i) said lipid carrier further comprises a glyceride moiety;
ii) said lipid carrier further comprises a ceramide moiety;
iii) said lipid carrier further comprises a second carbohydrate moiety;
iv) said lipid carrier further comprises one or more second polypeptides;
v) said lipid carrier further comprises a pharmaceutically active compound;
vi) said lipid carrier does not comprise one or both a recombinant polypeptide and a fusion polypeptide;
vii) is not expressed or synthetized by said microorganism;
viii) is at least partially expressed or synthetized by said microorganism;
ix) said lipid portion does not comprise one or more second polypeptides;
x) said lipid carrier does not comprise one or both a transmembrane polypeptide and a polypeptide membrane anchor domain;
xi) said lipid carrier is not susceptible to proteolysis;
xii) said lipid carrier is not immunogenic to a mammalian host;
xiii) said lipid carrier is immunogenic to a mammalian host;
xiv) said lipid carrier is not covalently bound to said cell membrane;
xv) said lipid carrier further comprises a glycolipid; and
xvi) said lipid carrier further comprises a lipopeptide.

38. The modified microorganism of claim 37, wherein one or more of (a) said glyceride moiety comprises at least one fatty acid; (b) said lipid portion of said lipid carrier comprises said glyceride moiety; (c) said ceramide moiety is composed of an amino alcohol and/or a fatty acid; (d) said lipid portion of said lipid carrier comprises said ceramide moiety; (e) said non-lipid portion of said lipid carrier comprises said second carbohydrate moiety; (f) said second carbohydrate moiety is ß- or α-linked to said ceramide moiety; (g) said second carbohydrate is not a monosaccharide or a disaccharide moiety; (h) said second carbohydrate is selected from the group consisting of an oligosaccharide and a polysaccharide; (i) a first sugar of the said second carbohydrate is selected from the group consisting of a galactose, a glucose, a mannose, a xylose, a neuraminic acid, a N-acetyl glucosamine, N-acetyl galactosamine and a galacturonic acid; (j) said non-lipid portion comprising said one or more second polypeptides; (k) said one or more second polypeptides is selected from the group consisting of an enzyme, a cytokine, a chemokine, a peptidomimetic compound, an antigen, an antibody, a fragment and a derivative thereof; (1) said non-lipid portion comprising said pharmaceutically active compound, (m) said recombinant polypeptide and/or fusion polypeptide is obtained by the means of artificial genetic manipulation; (n) said microorganism is capable of expressing or synthesizing a ceramide or sphingolipid moiety; (o) said lipid carrier is not immunogenic to a human, and (p) said lipid carrier is immunogenic to a human.

39. The modified microorganism of claim 31, wherein said lipid carrier is selected from the group consisting of:
i) Monosialotetrahexosylganglioside (GM1) or Monosialotetrahexosylganglioside red (GM1red) having the following formula: 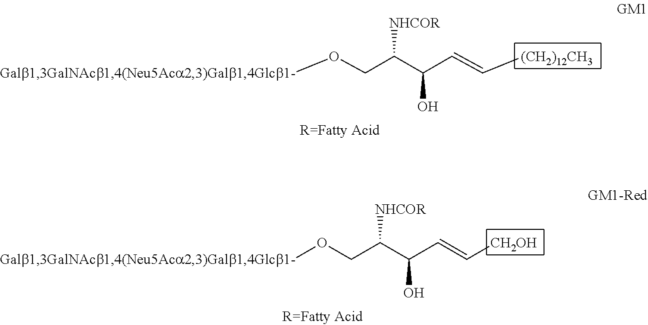
ii) Globotriaosylceramide (Gb3), a GM1-Gb3 chimera having the formula 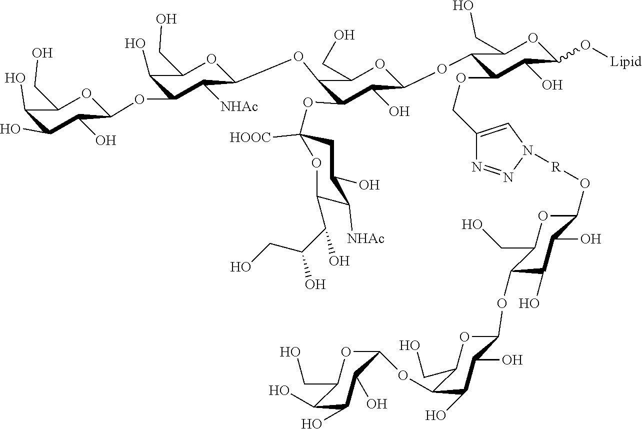
iii) Ganglioside GD1a,
iv) Gangliosides GM2, GD2, GD1b, GT1b, GT1c, GQ1c, GA1, GM1b,
v) Gangliosides GM3, GD3 and GT3,
vi) Gangliosides Gb4, Blood Group Type I, Type 2, Blood Group A, Blood Group B, Blood Group H, Blood Group H Type 1, Blood Group H Type 2, Blood Group H Type 3, Lewis y, Lewis a, Lewis b, Lewis x, H, Sialyl Lewis x, Sialyl Lewis a, Sialyl Lewis b, Sialyl Lewis x, alpha Gal epitope, Gal a1-3Galß1-4GlacNAc, Gal(α 1-4)Gal(ß1-4)GlcNAc-R, Gal(α 1-4)Gal(ß1-4)Glc, NAc-(ß1-3)Gal(ß1-4)Glc-R, Gal(α 1-4)Gal(ß1-4)GlcNAc(ß1-2) Man-R
vii) any one of the following:

| Name | Structure |
|---|---|
| 2-6 Sialyl i-Lewis x | Neu5Ac(α 2-6)Gal(β1-4)GlcNAc(β1-3)Gal(β1-4)[Fuc(α 1-3)]GlcNAc(β1-)-R |
| 3′-Sulfo Lewis a | HSO3(-3)Gal(β1-3)[Fuc(a1-4)]GlcNAc(β1-)-R |
| 3′-Sulfo Lewis x | HSO3(-3)Gal(β1-4)[Fuc(a1-3)]GlcNAc(β1-)-R |
| 6,6′-Disulfo Sialyl Lewis x | Neu5Ac(α 2-3)[HSO3(-6)]Gal(β1-4)[Fuc(α 1-3)][HSO3(-6)]GlcNAc(β1-)-R |
| 6-Sulfo Lewis x | Gal(β1-4)[Fuc(α 1-3)][HSO3(-6)]GlcNAc(β1-)-R |
| 6′-Sulfo Sialyl Lewis x | Neu5Ac(α 2-3)[HSO3(-6)]Gal(β1-4)[Fuc(α 1-3)]GlcNAc(β1-)-R |
| 6^{(GlcNAc)}-su-SLe^{x} | Neu5Acα2-3Galβ1-4(Fucα1-3)(6-O-Su)GlcNAcβ-R |
| 6′-Sia-6-Su-LacNAc | Neu5Acα2-6Galβ1-4(6-O-Su)GlcNAcβ-R |
| 6-Su-3′SiaLe^{c} | Neu5Acα2-3Galβ1-3(6-O-Su)GlcNAcβ-R |
| 6-Su-3′SLN | Neu5Acα2-3Galβ1-4(6-O-Su)GlcNAcβ-R |
| 3′SLN(Gc) | Neu5Gcα2-3Galβ1-4GlcNAcβ-R |
| 6′SLN(Gc) | Neu5Gcα2-6Galβ1-4GlcNAcβ-R |
| GlcNAcβ3′LacNAc | GlcNAcβ1-3Galβ1-4GlcNAcβ-R |
| Isomaltotriose | Glcα1-6Glca1-6Glcβ-R |
| Chitotriose | GlcNAcβ1-4GlcNAβ1-4GlcNAcβ-R |
| alpha Gal epitope, | Gal(α 1-3)Gal(β1-4)GlcNAc-R |
| Arthro | GlcNAcβ1,3Manβ1,4Glcβ-R |
| Aₜᵣᵢ | GalNAcα1-3(Fucα1-2)Galβ-R |
| Bₜᵣᵢ | Galα1-3(Fucα1-2)Galβ-R |
| Gal₂3,4-GlcNAc | Galβ1-4(Galβ1-3)GlcNAcβ-R |
| asialo-GM1,GA1 | DGalp(β1-3)DGalpNAc(β1-4)DGalp(β1-4)DGlcp(β1-1)-R |
| asialo-GM2,GA2 | DGalpNAc(β1-4)DGalp(β1-4)DGlcp(β1-1)-R |
| Blood Group A | GalNAc(α 1-3)[Fuc(α 1-2)]Gal(β1-)-R |
| Trisaccharide | |
| Blood Group A | GalNAc(α 1-3)[Fuc(α 1-2)]Gal(β1-3)GlcNAc(β1-)-R |
| Type 1 | |
| Blood Group A | GalNAc(α 1-3)[Fuc(α 1-2)]Gal(β1-3)[Fuc(α 1-4)]GlcNAc(β1-)-R |
| Type 1 (difucosyl) | |
| Blood Group A | GalNAc(α 1-3)[Fuc(α 1-2)]Gal(β1-4)GlcNAc(β1-)-R |
| Type 2 | |
| Blood Group A | GalNAc(α 1-3)[Fuc(α 1-2)]Gal(β1-4)[Fuc(α 1-3)]GlcNAc(β1-)-R |
| Type 2 (difucosyl) | |
| Blood Group A | GalNAc(α 1-3)[Fuc(α 1-2)]Gal(β1-3)GalNAc(α 1-)-R |
| Type 3 | |
| Blood Group A | GalNAc(α 1-3)[Fuc(α 1-2)]Gal(β1-3)GalNAc(β1-)-R |
| Type 4 | |
| Blood Group B | Gal(α 1-3)[Fuc (α 1-2)]Gal(β1-3)GlcNAc(β1-3)Gal-R, |
| Blood Group B | Gal(α 1-3)[Fuc(α 1-2)]Gal(β1-4)GlcNAc(β1-)-R |
| Type 2 | |
| Blood Group H | Fuc(α 1-2)Gal(β1-3)GlcNAc(β1-)-R |
| Type 1, | |
| Blood Group H | Fuc(α 1-2)Gal(β1-4)GlcNAc(β1-)-R |
| Type 2, | |
| Blood Group H | Fuc(α 1-2)Gal(β1-3)GalNAc(α 1-)-R |
| Type 3, | |
| Blood Group H, | Fuc(α 1-2)Gal(β1-)-R |
| Sia6′H (type 2) | Neu5Acα2-6(Fucα1-2)Galβ1-4GlcNAcβ-R |
| 6-LacNAc-TF | Galβ1-4GlcNAcβ1-6(Galβ1-3)GalNAcα-R |
| C-Series Ganglio- | R-Gal(β1-3)GalNAc(β1-4)[Neu5Ac(α 2-8)Neu5Ac(α 2-8)Neu5Ac(α 2- |
| sides Oligo- | 3)]Gal(β1-4)Glc(β1-1)-R |
| saccharide/Ganglio- | |
| tetraosyl Core | |
| Structure | |
| C-Series Ganglio- | Neu5Ac(α 2-8)Neu5Ac(α 2-8)Neu5Ac(α 2-3)Gal-R |
| sides Oligo- | |
| saccharide/Hemato- | |
| or Ganglio-Type | |
| Cyclic Sialyl 6-Sulfo Lewis x | cyclicNeu5Ac(α 2-3)Gal(b1-4)[Fuc(α 1-3)][HSO3(-6)]GlcNAc-R |
| Dimeric Lewis x | Gal(β1-4)[Fuc(α 1-3)]GlcNAc(β1-3)Gal(b1-4)[Fuc(α 1-3)]GlcNAc(β1-)-R |
| Disialyl Lewis a | Neu5Ac(α 2-3)Gal(β1-3)[Neu5Ac(α 2-6)][Fuc(α 1-4)]GlcNAc(β1-)-R |
| Disialyl Lewis c | Neu5Ac(α 2-3)Gal(β1-3)[Neu5Ac(α 2-6)]GlcNAc(β1-)-R |
| F1 Alpha | Gal(b1-4)GlcNAc(b1-6)GalNAc(α 1-)Ser/Thr |
| fucosyl GM1 | (Fucal-2Galβ1-3GalNAcβ1-4[NeuAca2-3]-Galβ1-4Glcβ1-I-R |
| GA1: (Gg4Cer) | Galβ1,3GalNAcβ1,4Galβ1,4Glcβ1-R |
| GA2: (Gg3Cer) | GalNAcβ1,4Galβ1,4Glcβ1-R |
| Gal a1-3Galβ1-4GlacNAc | Gal α 1-3Galβ1-4GlacNAc-R |
| Galili,Gala-3′LacNAc | Galα1-3Galβ1-4GlcNAcβ-R |
| Gala | Gal α 1,4Galβ-R |
| GalCer: | Galβ1-R |
| GalNAc-GD1a: | GalNAcβ1,4Galβ1,3GalNAcβ1,4(Neu5Acα2,3)Galβ1,4Glcβ1-R |
| (IV3Neu5AcII3Neu5AcGg5Cer) | |
| Ganglio | Galβ1,3GalNAcβ1,4Galβ1,4Glcβ-R |
| GbOse3Cer: | Galα1,4Galβ1,4Glcβ1-R |
| (Gb3Cer) | |
| GbOse4Cer | GalNAcβ1,3Galα1,4Galβ1,4Glcβ1-R |
| GD1a: | Neu5Acα2,3Galβ1,3GalNAcβ1,4(Neu5Acα2,3)Galβ1,4Glcβ1-R |
| (IV3Neu5AcII3Neu5AcGg4Cer) | |
| GD1b: | Galβ1,3GalNAcβ1,4(Neu5Acα2,8Neu5Acα2,3)Galβ1,4Glcβ1-R |
| (II3(Neu5Ac)2Gg4Cer) | |
| GD1b-lactone: | II3[Neu5Ac-(2-8,1-9)-Neu5Ac]Gg4-R |
| GD1c: | Neu5Acα2,8Neu5Acα2,3Galβ1,3GalNAcβ1,4Galβ1,4Glcβ1-R |
| (IV3(Neu5Ac)2Gg4Cer) | |
| GD1α: | Neu5Ac α2,3Galβ1,3(Neu5Acα2,6)GalNAcβ1,4Galβ1,4Glcβ1-R |
| (IV3Neu5AcIII6Neu5AcGg4Cer) | |
| GD2: | GalNAcβ1,4(Neu5Acα2,8Neu5Acα2,3)Galβ1,4Glcβ1-R |
| (II3(Neu5Ac)2Gg3Cer) | |
| GD3: | Neu5Acα2,8Neu5Acα2,3Galβ1,4Glcβ1-R |
| (II3(Neu5Ac)2LacCer) | |
| GGal | Neu5Ac(α 2-3)DGalp(β1-1)-R |
| GlcCer: | Glcβ1-R |
| Globo | GalNAcβ1,3Gala1,4Galβ1,4Glcβ-R |
| Globo-H | Fuc α 2Galβ3GalNAcβ3Gal α 4Galβ4Glcβ1-R |
| Gb5 | Gal(β1-3)GalNAc(β 1-3)Gal(a 1-4)Gal(β 1-4)Glc-R |
| Gb5 | Galβ3GalNAcβ3Galα4Galβ4Glcβ1-R |
| monosialyl-Gb5 | SAα3Galβ3GalNAcβ3Galα4Galβ4Glcβ1-R |
| disialyl-Gb5 | SAα3Galβ3GalNAcβ3(SAa2-3)Galα4Galβ4Glcβ1-R |
| iso-Gb3 | Galα3Galβ4Glcβ1-R |
| iso-Gb4 | GalNAcβ3Galα3Galβ4Glcβ1-R |
| Forssman | GalNAcα3GalNAcβ3Galα4Galβ4Glcβ1-R |
| GM1a: r | Galβ1,3GalNAcβ1,4(Neu5Acα2,3)Galβ1,4Glcβ1-R |
| (II3Neu5AcGg4Cer) | |
| GM1b: | Neu5Acα2,3Galβ1,3GalNAcβ1,4Galβ1,4Glcβ1-R |
| (IV3Neu5AcGg4Cer) | |
| GM2: | GalNAcβ1,4(Neu5Acα2,3)Galβ1,4Glcβ1-R |
| (II3Neu5AcGg3Cer) | |
| GM2b | Neu5Ac(α 2-8)Neu5Ac(α 2-3)DGalp(β1-4)DGlcp(β1-1)-R |
| GM3: | Neu5Acα2,3Galβ1,4Glcβ1-R |
| (II3Neu5AcLacCer) | |
| GM4: | Neu5Acα2-3Galβ1-R |
| (I3Neu5AcαGalCer) | |
| GP1c: | Neu5Acα2,8Neu5Acα2,3Galβ1,3GalNAcβ1,4 |
| (IV3(Neu5Ac)2II3 | (Neu5Acα2,8Neu5Acα2,8Neu5Acα2,3)Galβ1,4Glcβ1-R |
| (Neu5Ac)3Gg4Cer) | |
| GP1cα: | Neu5Ac |
| (IV3Neu5AcIII6Neu5Ac,II3(Neu5Ac)3Gg4Cer) | α2,3Galβ1,3(Neu5Acα2,6)GalNAcβ1,4(Neu5Acα2,8Neu5Acα2,8Neu5Acα2,3)Galβ1,4Glcβ1-R |
| GQ1b: | Neu5Acα2,8Neu5Acα2,3Galβ1,3GalNAcβ1,4(Neu5Acα2,8Neu5Acα2,3)Galβ1,4Glcβ1-R |
| (IV3(Neu5Ac)2II3 | |
| (Neu5Ac)2Gg4Cer) | |
| GQ1bα: | Neu5Ac |
| (IV3(Neu5Ac)2III6(Neu5Ac)2Gg4Cer) | α2,3Galβ1,3(Neu5Acα2,6)GalNAcβ1,4(Neu5Acα2,8Neu5Acα2,3)Galβ1,4Glcβ1-R |
| GQ1c: | Neu5Acα2,3Galβ1,3GalNAcβ1,4(Neu5Acα2,8Neu5Acα2,8Neu5Acα2,3)Galβ1,4Glcβ1-R |
| (IV3Neu5AcII3(Neu5Ac)3Gg4Cer) | |
| GT1,GT1b | Neu5Ac(α 2-3)DGalp(β1-3)DGalNAc(β1-4)[Neu5Ac(α 2-8)Neu5Ac(α |
| | 2-3)]DGalp(β1-4)DGlcp(β1-1)-R |
| GT1a: | Neu5Acα2,8Neu5Acα2,3Galβ1,3GalNAcβ1,4(Neu5Acα2,3)Galβ1,4Glcβ1-R |
| N(V3(Neu5Ac)2II3Neu5AcGg4Cer) | |
| GT1b: | Neu5Acα2,3Galβ1,3GalNAcβ1,4(Neu5Acα2,8Neu5Acα2,3) Galβ1,4Glcβ1-R |
| (IV3Neu5AcII3(Neu5Ac)2Gg4Cer) | |
| GT1c: | Galβ1,3GalNAcβ1,4(Neu5Acα2,8Neu5Acα2,8Neu5Acα2,3)Galβ1,4Glcβ1-R |
| (II3(Neu5Ac)3Gg4Cer) | |
| GT1α: | Neu5Ac α2,3Galβ1,3(Neu5Acα2,6)GalNAcβ1,4(Neu5Acα2,3)Galβ1,4Glcβ1-R |
| (IV3Neu5AcIII6(Neu5Ac)2Gg4Cer) | |
| GT2: | GalNAcβ1,4(Neu5Acα2,8Neu5Acα2,8Neu5Acα2,3)Galβ1,4Glcβ1-R |
| GT3: | Neu5Acα2,8Neu5Acα2,8Neu5Acα2,3Galβ1,4Glcβ1-R |
| (II3(NeuAc)3LacCer) | |
| Internal Lewis x | Gal(b1-4)GlcNAc(b1-3)Gal(b1-4)[Fuc(a1-3)]GlcNAc(b1-3)Gal(b1-4)GlcNAc(b1- |
| | 3)Gal(b1-4)GlcNAc(b1-3)Gal(b1-4)Glc(b1-1)-R |
| Isoglobo | GalNAcβ1,3Gala1,3Galβ1,4Glcβ-R |
| LacCer: | Galβ1,4Glcβ1-R |
| Lacto | Galβ1,3GlcNAcβ1,3Galβ1,4Glcβ-R |
| Lewis a, | Gal(β1-3)[Fuc(α 1-4)]GlcNAc(β1-)-R |
| Lewis b, | Fuc(α 1-2)Gal(β1-3)[Fuc(α 1-4)]GlcNAc(β1-)-R |
| Lewis x,H, | Gal(β1-4)[Fuc(α 1-3)]GlcNAc(β1-)-R |
| Lewis y, | Fuc(α 1-2)Gal(β1-4)[Fuc(α 1-3)]GlcNAc(β1-)-R |
| Mollu | Fuc α 1,4GlcNAcβ1,2Man α 1,3Manβ 1,4Glcβ-R |
| Muco | Galβ1,3Galβ1,4Galβ1,4Glcβ-R |
| N-Acetyl GD3 | Neu5Ac(α 2-8)Neu5Ac(α 2-3)Gal(b1-4)Glc(b1-1)-R |
| Neogala | Galβ 1,6Galβ 1,6Galβ-R |
| Neolacto | Galβ1,4GlcNAcβ1,3Galβ1,4Glcβ-R |
| Neu5Ac(a2- | Neu5Ac(α 2-3)Gal(β1-)-R |
| 3)Gal(b1) | |
| Neu5Ac(a2- | Neu5Ac(α 2-3)Gal(β1-3)GalNAc-R |
| 3)Gal(b1- | |
| 3)GalNAc | |
| Neu5Ac(a2- | Neu5Ac(α 2-8)Neu5Ac(α 2-3)Gal-R |
| 8)Neu5Ac(a2- | |
| 3)Gal | |
| (Sia)₃ | Neu5Acα2-8Neu5Acα2-8Neu5Acα-R |
| 3′-SL | Neu5Acα2-3Galβ1-4Glcβ-R |
| N-Glycolyl GM3 | Neu5Gc(α 2-3)Gal(β1-4)Glc(β1-1)-R |
| NOR1 | Gal(α 1-4)GalNAc(β 1-3)Gal(α a 1-4)Gal(β 1-4)Glc-R |
| NOR2 | Gal(α 1-4)GalNAc-(β 1-3)Gal(α 1-4)GalNAc(β 1-3)Gal(α 1-4)Gal(β 1-4)Glc-R |
| NOT int | GalNAc(β 1-3)Gal(α 1-4)GalNAc(β1-3)Gal-(α 1-4)Gal(β 1-4)Glc-R |
| O antigen | Fuc (α 1-2)Gal(β1-3)GlcNAc(β1-3)Gal-R, |
| OAc-GT1b | Neu5Ac(α 2-3)DGalp(β1-3)DGalNAc(β1-4)XNeu5Ac9Ac(α 2-8)Neu5Ac(α 2- |
| | 3)]DGalp(β-4)DGlcp(β1-1)-R |
| P antigen (Gb4) | Gal(α 1-4)Gal-(β 1-4)GlcNAc(β 1-3)Gal(β 1-4)Glc-R |
| Pk Antigen (Gb3) | Gal(α 1-4)Gal(β1-4)Glc-R |
| Pi | Galα1-4Galβ1-4GlcNAcβ-R |
| Schisto | GalNAcβ1,4Glcβ-R |
| Sialyl Lewis a, | Neu5Ac(α 2-3)Gal(β1-3)[Fuc(α 1-4)]GlcNAc(β1-)-R |
| Sialyl Lewis c | Neu5Ac(α 2-3)Gal(β1-3)GlcNAc(β1-)-R |
| Sialyl Lewis x, | Neu5Ac(α 2-3)Gal(β1-4)[Fuc(α 1-3)]GlcNAc(β1-3)Gal(β1-4)GlcNAc(β1-3)Gal(β1-)-R |
| Sialyl Lewis x, | Neu5Ac(α 2-3)Gal(β1-4)[Fuc(α 1-3)]GlcNAc(β1-)-R |
| Sialyl Lewis x-i | Neu5Ac(α 2-3)Gal(β1-4)[Fuc(α 1-3)]GlcNAc(β1-3)Gal(β1-4)GlcNAc(β1-3)Gal(β1-)-R |
| LNT | Galβ1-3GlcNAcβ1-3Galβ1-4Glcβ-R |
| LNnT | Galβ1-4GlcNAcβ1-3Galβ1-4Glcβ-R |
| Sialyl-TF | Neu5Ac(α 2-6) Gal (β1-3) α GalNAc-R, |
| sialyl-Tn | Neu5Ac(α 2-6)GalNAc-R, |
| sLac | NeuAc-Gal0-3GicNAcB-3GalB-4Glc-R |
| Spirometo | Galβ1,4Glcβ1,3Galβ-R |
| Sulfatide: | Sulfate3Galβ1-R |
| TF/Core-1 | α Gal (β1-3)aGalNAc-R, |
| 3′-sialyl-TF | Neu5Acα2-3Galβ1-3GalNAcα-R |
| Tn | α GalNAc-R |
| 6-SiaβTF | Neu5Acβ2-6(Galβ1-3)GalNAcα-R |
| 3-LacNAc-Tₙ | Galβ1-4GlcNAβ1-3GalNAcα-R |
| 6-LacNAc-Tₙ | Galβ1-4GlcNAβ1-6GalNAcα-R |
| 6′SLN | Neu5Acα2-6Galβ1-4GlcNAcβ-R |
| Core 2 | GlcNAcβ1-6(Galβ1-3)GalNAcα-R |
| Core 4 | GlcNAcβ1-3(GlcNAcβ1-6)GalNAcα-R |
| Trifucosyl-Lewis b Antigen | Fuc(α 1-2)Gal(b1-3)[Fuc(α 1-4)]GlcNAc(b1-3)Gal(b1-3)[Fuc(α 1-4)]GlcNAc(b1-)-R |
| Trifucosyl-Lewis y Antigen | Fuc(α 1-2)Gal(b1-4)[Fuc(α 1-3)]GlcNAc(b1-3)Gal(b1-4)[Fuc(α 1-3)]GlcNAc(b1-)-R |
| Type 1 | (GalNAc(α 1-3)[Fuc(α 1-2)]Gal(β1-3)GlcNAc[Fuc(α 1-4)]-R), |
| Type 1 A | GalNAc(α1-3)[Fuc(α 1-2)]Gal(β1-3)GlcNAc(β1-3)Gal-R, |
| Type 2 A | GalNAc(α1-3)[Fuc(α 1-2)]Gal(β1-4)GlcNAc-R, |
| Type 3 A | GalNAc(α1-3)[Fuc(α 1-2)]Gal(β1-3)GalNAc(β1-3)Gal[Fucα 1-2)-R, |
| Type 4 A | GalNAc(α1-3)[Fuc(α 1-2)]Gal(β1-3)GalNAc(β1-3)Gal(a 1-4)Gal(β 1-4)Glc--R, |
| VIM-2 | Neu5Ac(α 2-3)Gal(β1-4)GlcNAc(β1-3)Gal(β1-4)[Fuc(α-3)]GlcNAc(β1-)-R |
| Type 4 A | GalNAc(α1-3)[Fuc(α 1-2)]Gal(β1-3)GalNAc(β1-3)Gal(a 1-4)Gal(β 1-4)Glc--R, |
| Man₃ | Manα1-3(Manα1-6)Manα-? |
| 3′SLN | Neu5Acα2-3Galβ1-4GlcNAcβ-R |
| 6′SL | Neu5Acα2-6Galβ1-4Glcβ-R |
| Human milk oligosaccharide | see WO 2012/092153, WO 2010/120682, WO 2005/055944, U.S. Pat. No. 5,945,314 |

wherein R is one or more of the following: a first carbohydrate/s, a second carbohydrate/s, a peptide/s, a lipid/s, a linker/s and a chemical compound/s or substance/s or molecule/s; or R comprises one or more of the following: a first carbohydrate/s, a second carbohydrate/s, a peptide/s, a lipid/s, a linker/s or a chemical compound/s or substance/s or molecule/s;
viii) any one of lipid carriers of i)-vii) further coupled to TF disaccharide, Core-1 structure, Tn monosaccharide, Sialyl-TF mono- or disialylated, Sialyl-Tn, Polysialic acid, or mannose-6-phosphate moiety;
ix) any one of lipid carriers of i)-viii) further coupled to N-Glycan or O-glycan moiety;
x) any one of lipid carriers of i)-ix) further coupled to a second carbohydrate moiety;
xi) a truncated or elongated derivative of any one of lipid carriers of i)-x);
xii) a phosphorylated, sulfated or acetylated derivative of any one of lipid carriers of i)-xi);
xiii) derivatives and analogs of any of (i)-(xii).

40. The modified microorganism of claim 31, wherein said microorganism is generated from a naturally-occurring microorganism.

41. The modified microorganism of claim 40, wherein said naturally-occurring microorganism is obtainable from one or more of the following sources:
i) microflora or microbiota of an animal;
ii) soil microflora or microbiota;
iii) microbiota from plants; and
iv) microbiota from food.

42. The modified microorganism of claim 41, wherein said naturally-occurring microorganism is obtained from one or more samples from a naturally-occurring microorganism selected from the group consisting of microflora of (a) an animal vertebral organism, microbiota of said animal vertebral organism; (b) microbiota of a digestive system of said vertebral organism, microflora of said digestive system of said vertebral organism, microbiota of a urogenital system of said vertebral organism, microflora of said urogenital system of said vertebral organism, skin microflora of said vertebral organism, (c) microflora of a human, microbiota of a human, and (d) microbiota from leaves, microbiota from fruits, microbiota from berries, and microbiota from marine ecosystems; where said naturally-occurring microorganism is obtained from a sample selected from the group consisting of gut, feces, oral cavity, nasal cavity, vagina, lung, sputum, mucus source, and urine of said vertebral organism.

43. The modified microorganism of claim 31, wherein said microorganism is selected from the group consisting of:
i) a bacterium;
ii) a fungus; and
iii) a protozoan.

44. The modified microorganism of claim 43, wherein one or more of (a) said bacterium is selected from the group consisting of a gram-positive bacteria, a gram-negative bacteria, non-pathogenic and opportunistic pathogen; (b) said gram-positive bacteria is selected from the genera consisting of Lactobacillus, Bifidobacterium, Clostridium, Enterococcus, Pediococcus and Streptococcus, Lactobacillus paracasei, Lactobacillus reuteri; (c) said fungus is selected from the group consisting of Candida yeasts, Saccharomyces yeasts selected from the group consisting of Saccharomyces boulardii, S. cerevisiae, S. pastorianus, and Schizosaccharomyces pombe and yeasts in the family Dipodascaceae, selected from the group consisting of Galactomyces, Geotrichum and Saprochaete.

45. A composition comprising one or more of a modified microorganism comprising:
i) a cell, wherein said cell does not comprise a mycomembrane; and
ii) a heterologous lipid carrier, said lipid carrier comprising:
a) a lipid portion, wherein said lipid portion is at least partially associated with an exterior surface of said cell, wherein said lipid portion comprises one or both a ceramide-like glycolipid moiety and a fatty acid moiety; and
b) a non-lipid portion, wherein said microorganism is capable of one or both locating and displaying said non-lipid portion or fragment thereof onto the exterior surface of said cell and wherein said heterologous lipid carrier is not alpha-galactosylceramide.

46. The composition of claim 45, wherein said composition is a pharmaceutical, a diagnostic, a probiotic or a prebiotic composition.

47. A vaccine or adjuvant comprising said composition of claim 46.

48. A method for producing or isolating a modified microorganism comprising:
i) a cell, wherein said cell does not comprise a mycomembrane; and
ii) a heterologous lipid carrier, said lipid carrier comprising:
a) a lipid portion, wherein said lipid portion is at least partially associated with an exterior surface of said cell, wherein said lipid portion comprises one or both a ceramide-like glycolipid moiety and a fatty acid moiety; and
b) a non-lipid portion, wherein said microorganism is capable of one or both locating and displaying said non-lipid portion or fragment thereof onto the exterior surface of said cell;
and wherein said heterologous lipid carrier is not alpha-galactosylceramide;
said method comprising:
iii) culturing:
a) a microorganism or mixture of microorganisms obtainable from one or more of the following sources:
aa′) microflora or microbiota of an animal,
bb′) soil microflora or microbiota;
cc′) microbiota of plants;
dd′) microbiota from food;
in growth medium.

49. The method for producing or isolating a modified microorganism of claim 48, wherein one or more of (a) said exterior surface of said cell comprises: a cell wall and/or a cell membrane and/or an outer cell membrane and/or a polysaccharide; (b) said lipid portion is at least partially incorporated and/or adhered and/or bound to said exterior surface of said cell; (c) said non-lipid portion comprises: a first carbohydrate moiety, a lipopeptide moiety, a linker, a chemical compound, or a first peptide moiety; (d) said modified microorganism further comprises a heterologous steroid moiety selected from the group consisting of cholesterol, a cholesterol derivative and a cholesterol analog thereof; (e) said microorganism or mixture of microorganisms are naturally-occurring.

50. The method for producing or isolating a modified microorganism of claim 48, wherein said naturally-occurring microorganism is obtained from one or more samples from a naturally-occurring microorganism selected from the group consisting of (a) microflora of an animal vertebral organism, microbiota of said animal vertebral organism; (b) microbiota of a digestive system of said vertebral organism, microflora of said digestive system of said vertebral organism, microbiota of a urogenital system of said vertebral organism, microflora of said urogenital system of said vertebral organism, skin microflora of said vertebral organism, (c) microflora of a human, microbiota of a human, and (d) microbiota from leaves, microbiota from fruits, microbiota from berries, and microbiota from marine ecosystems; where said naturally-occurring microorganism is obtained from a sample selected from the group consisting of gut, feces, oral cavity, nasal cavity, vagina, lung, sputum, mucus source, and urine of said vertebral organism.
